(19) United States
(12) Patent Application Publication
(10) Pub. No.: US 2026/0193645 A1
(43) Pub. Date: Jul. 9, 2026

(54) COMPOSITIONS AND METHODS FOR BASE EDITING A PHENYLALANINE HYDROXYLASE POLYNUCLEOTIDE

(71) Applicant: Beam Therapeutics Inc., Cambridge, MA (US)

(72) Inventors: Tanggis Bohnuud, Cambridge, MA (US); Mandy Sze Lee, Cambridge, MA (US); Yi Yu, Cambridge, MA (US)

(73) Assignee: Beam Therapeutics Inc., Cambridge, MA (US)

(21) Appl. No.: 19/556,903

(22) Filed: Mar. 4, 2026

Related U.S. Application Data
(63) Continuation of application No. PCT/US2024/046693, filed on Sep. 13, 2024.
(60) Provisional application No. 63/582,942, filed on Sep. 15, 2023.

(51) Int. Cl. C12N 15/11; A61P 3/00; C12N 9/22; C12N 9/78; C12N 15/88; C12N 15/90

(52) U.S. Cl. CPC C12N 15/11; A61P 3/00; C12N 9/226; C12N 9/78; C12N 15/88; C12N 15/907; C12Y 305/04004; C12N 2310/20

(57) ABSTRACT

Compositions and methods for treating phenylketonuria by introducing one or more alterations into a phenylalanine hydroxylase (PAH) polynucleotide in a cell. In particular embodiments, the disclosure provides a base editor system (e.g., a fusion protein or complex comprising a programable DNA binding protein, a nucleobase editor, and gRNA) for modifying a PAH polynucleotide, where the alteration is associated with an increase in the level or activity of a PAH polypeptide encoded by the polynucleotide.

### CROSS REFERENCE TO RELATED APPLICATIONS

This application is a continuation under 35 U.S.C. § 111 (a) of PCT International Patent Application No. PCT/US2024/046693, filed Sep. 13, 2024, designating the United States and published in English, which claims priority to and the benefit of U.S. Provisional Application No. 63/582,942, filed Sep. 15, 2023, the entire contents of each of which are incorporated by reference herein.

### SEQUENCE LISTING

This application contains a Sequence Listing which has been submitted electronically in XML format and is hereby incorporated by reference in its entirety. The Sequence Listing XML file, created on Sep. 11, 2024, is named 180802-055902PCT_SL.xml and is 1,311,624 bytes in size.

### BACKGROUND

Phenylalanine hydroxylase (PAH) deficiency prevents the conversion of phenylalanine (Phe) to tyrosine. Untreated phenylketonuria (PKU) patients can have very high blood Phe levels of more than 1200 μmol/L (normal Phe levels are less than 120 μmol/L), with neurocognitive and neuropsychiatric sequelae. A strict low-Phe diet is the mainstay of treatment, with the goal of maintaining Phe levels of 120-360 μmol/L, which still exceed the physiologic range. However, many PKU patients find it challenging to adhere to the unpalatable and cost-prohibitive diet.

There are only two approved medical therapies for treating PKU. The first is sapropterin, an oral medication that serves as a cofactor of the PAH protein and can improve the activity of some mutant forms of PAH. However, the P281L variant, one of the most common PKU pathogenic variants with its highest prevalence in populations in the Middle East, Europe, and Russia (e.g., present in 14.8% of PKU patients in the Netherlands, 11.2% in Turkey, 10.8% in Portugal, 10.3% in Italy, and 9.4% in Germany), is unresponsive to sapropterin (i.e., blood Phe levels do not improve with sapropterin treatment). The second is pegvaliase, an injectable enzyme that acts directly to catabolize Phe. However, pegvaliase carries a substantial risk of anaphylaxis and has a black box warning from the U.S. Food and Drug Administration on its label for that reason. In addition, the dosing regimen is complex, starting with once-weekly injections and slowly up-titrating to once-daily injections, and it is available in the U.S. only through a Risk Evaluation and Mitigation Strategy program. In a long-term study, patients on pegvaliase achieved a mean 51% Phe reduction at 1 year after initiation (1233-565 μmol/L). Thus, a safe one-time therapy that would durably, even permanently, normalize blood Phe levels would be a vastly superior treatment option over existing alternatives.

Therefore, there is a present need for improved compositions and methods for treatment of PKU.

### SUMMARY

As described below, the disclosure features compositions and methods for treating phenylketonuria by introducing one or more alterations into a phenylalanine hydroxylase (PAH) polynucleotide in a cell. In particular embodiments, the disclosure provides a base editor system (e.g., a fusion protein or complex containing a programable DNA binding protein, a nucleobase editor, and gRNA) for modifying a PAH polynucleotide, where the alteration is associated with an increase in activity of a PAH polypeptide encoded by the polynucleotide.

In one aspect, the disclosure features a method of editing a nucleobase of an phenylalanine hydroxylase (PAH) polynucleotide. The method involves contacting the PAH polynucleotide with a guide RNA, or a polynucleotide encoding the guide RNA, and a base editor containing a fusion protein or a protein complex containing a nucleic acid programmable DNA binding protein (napDNAbp) domain and a deaminase domain, or one or more polynucleotides encoding the base editor. The guide RNA targets the base editor to effect an alteration to a nucleobase in codon 408 of the PAH polynucleotide.

In one aspect, the disclosure features a method of editing a nucleobase of an phenylalanine hydroxylase (PAH) polynucleotide. The method involves contacting the PAH polynucleotide with a guide RNA, or a polynucleotide encoding the guide RNA, and a base editor containing a fusion protein or a protein complex containing a nucleic acid programmable DNA binding protein (napDNAbp) domain and a deaminase domain, or one or more polynucleotides encoding the base editor. The guide RNA targets the base editor to effect an alteration to a nucleobase in codon 408 of the PAH polynucleotide. The deaminase domain contains the following amino acid sequence with one or more amino acid alterations MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a fragment thereof lacking only the N-terminal methionine. The one or more amino acid alterations contains a set of alterations selected from one or more of: a) I76Y, V82T, Y123H, Y147R, and Q154R; b) I76Y, V82T, Y123H, Y147R, F149Y, and Q154R; and c) I76Y, V82T, Y123H, Y147D, F149Y, Q154R, T166I, and D167N.

In another aspect, the disclosure features a method of treating phenylketonuria (PKU) in a subject in need thereof. The method involves administering to a cell of the subject a base editor system containing a base editor containing fusion protein or protein complex containing a nucleic acid programmable DNA binding protein (napDNAbp) domain and a deaminase domain, or one or more polynucleotides encoding the base editor, and a guide RNA that targets the base editor to effect an alteration to a nucleobase in codon 408 of a phenylalanine hydroxylase (PAH) polynucleotide in the cell, or a polynucleotide encoding the guide RNA, thereby treating hypertension in the subject.

In another aspect, the disclosure features a method of treating phenylketonuria (PKU) in a subject in need thereof. The method involves administering to a cell of the subject a base editor system containing a base editor containing fusion protein or protein complex containing a nucleic acid programmable DNA binding protein (napDNAbp) domain and a deaminase domain, or one or more polynucleotides encoding the base editor, and a guide RNA that targets the base editor to effect an alteration to a nucleobase in codon 408 of a phenylalanine hydroxylase (PAH) polynucleotide in the cell, or a polynucleotide encoding the guide RNA, thereby treating hypertension in the subject. The deaminase domain contains the following amino acid sequence with one or more amino acid alterations MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a fragment thereof lacking only the N-terminal methionine. The one or more amino acid alterations contains a set of alterations selected from one or more of: a) I76Y, V82T, Y123H, Y147R, and Q154R; b) I76Y, V82T, Y123H, Y147R, F149Y, and Q154R; and c) I76Y, V82T, Y123H, Y147D, F149Y, Q154R, T166I, and D167N.

In another aspect, the disclosure features a modified cell containing an alteration in a nucleobase of an PAH polynucleotide. The alteration is prepared by the method of any one of the aspects of the disclosure, or embodiments thereof. The alteration increases activity of the encoded PAH polypeptide as compared to a control cell without the alteration.

In another aspect, the disclosure features a base editor system containing a base editor or one or more polynucleotides encoding the base editor. The base editor contains a nucleic acid programmable DNA binding protein domain (napDNAbp) and a deaminase domain, and a guide RNA that targets the base editor to effect an alteration to a nucleobase in codon 408 of the PAH polynucleotide.

In another aspect, the disclosure features a base editor system containing a base editor or one or more polynucleotides encoding the base editor. The base editor contains a nucleic acid programmable DNA binding protein domain (napDNAbp) and a deaminase domain, and a guide RNA that targets the base editor to effect an alteration to a nucleobase in codon 408 of the PAH polynucleotide. The deaminase domain contains the following amino acid sequence with one or more amino acid alterations MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a fragment thereof lacking only the N-terminal methionine. The one or more amino acid alterations contains a set of alterations selected from one or more of: a) I76Y, V82T, Y123H, Y147R, and Q154R; b) I76Y, V82T, Y123H, Y147R, F149Y, and Q154R; and c) I76Y, V82T, Y123H, Y147D, F149Y, Q154R, T166I, and D167N.

In another aspect, the disclosure features a set of polynucleotides encoding the base editor system of any aspect of the disclosure, or embodiments thereof, or a component thereof.

In another aspect, the disclosure features a lipid nanoparticle containing the base editor system or set of polynucleotides of any aspect of the disclosure or embodiments thereof.

In another aspect, the disclosure features a pharmaceutical composition containing the base editor system, the set of polynucleotides, or the lipid nanoparticle of any aspect of the disclosure, or embodiments thereof, and a pharmaceutically acceptable excipient.

In another aspect, the disclosure features a kit containing the base editor system of any, the set of polynucleotides, the lipid nanoparticle, or the pharmaceutical composition, of any aspect of the disclosure, or embodiments thereof, for use in the method of any aspect of the disclosure, or embodiments thereof, where the kit further contains a container.

In another aspect, the disclosure features a guide RNA containing a sequence listed in Table 1.

In any aspect of the disclosure, or embodiments thereof, the alteration to the nucleobase in codon 408 of the PAH polynucleotide results in a W408R amino acid alteration in a PAH polypeptide encoded by the PAH polynucleotide.

In any aspect of the disclosure, or embodiments thereof, the guide RNA contains a spacer containing at least 10 contiguous nucleotides of a spacer sequence selected from those listed in Table 1. In any aspect of the disclosure, or embodiments thereof, the guide RNA contains a spacer containing a spacer sequence selected from those listed in Table 1. In any aspect of the disclosure, or embodiments thereof, the guide RNA containing a guide sequence selected from those listed in Table 1.

In any aspect of the disclosure, or embodiments thereof, the deaminase domain contains: a) a TadA*8.20 domain, b) a TadA*8.20 domain containing a V82T amino acid alteration, c) a TadA*8.20 domain containing the amino acid alterations V82T and F149Y, or d) a TadA*8.20 domain containing the amino acid alterations V82T, R147D, F149Y, T166I, and D167N.

In any aspect of the disclosure, or embodiments thereof, the napDNAbp domain is selected from the one or more of an SpCas9, an SaCas9, an iSpymac, and an NmeCas9. In any aspect of the disclosure, or embodiments thereof, the napDNAbp domain binds a protospacer adjacent motif (PAM) selected from the one or more of NRN, NAA, NCA, NGC, NRCH, NG, NRTH, NNNRRT, and NGG, where “N” is A, C, G, or T, “R” is A or G, and “H” is A, C, or T.

In any aspect of the disclosure, or embodiments thereof, the base editor contains an amino acid sequence having at least 85% sequence identity to a sequence selected from those listed in Table 2. In any aspect of the disclosure, or embodiments thereof, the base editor contains an amino acid sequence selected from those listed in Table 2. In any aspect of the disclosure, or embodiments thereof, the one or more polynucleotides encoding the base editor contain an RNA sequence encoding a polypeptide having at least 85% sequence identity to an amino acid sequence selected from those listed in Table 2. In any aspect of the disclosure, or embodiments thereof, the one or more polynucleotides encoding the base editor contain an RNA encoding a polypeptide having an amino acid sequence selected from those listed in Table 2.

In any aspect of the disclosure, or embodiments thereof, the deaminase is inserted within the napDNAbp domain. In any aspect of the disclosure, or embodiments thereof, the deaminase is an adenosine deaminase.

In any aspect of the disclosure, or embodiments thereof, the PAH polynucleotide is in a cell. In any aspect of the disclosure, or embodiments thereof, the cell is in vivo or in vitro. In any aspect of the disclosure, or embodiments thereof, the cell is a mammalian cell. In any aspect of the disclosure, or embodiments thereof, the cell is a liver cell. In any aspect of the disclosure, or embodiments thereof, the cell is a hepatocyte.

In any aspect of the disclosure, or embodiments thereof, alteration of the nucleobase is associated with an increase in phenylalanine hydroxylase activity in the subject or with a reduction in phenylalanine levels in a biological sample of the subject. In any aspect of the disclosure, or embodiments thereof, the phenylalanine levels in the biological sample are reduced by at least about 10, 25, 50, or 75% or where the levels are reduced to about 2-6 mg/dL or less.

In any aspect of the disclosure, or embodiments thereof, the base editor system is administered to the subject using a lipid nanoparticle containing the guide RNA and an mRNA molecule encoding the base editor.

### Definitions

Unless defined otherwise, all technical and scientific terms used herein have the meaning commonly understood by a person skilled in the art to which this disclosure belongs. The following references provide one of skill with a general definition of many of the terms used in this disclosure: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “phenylalanine hydroxylase (PAH) polypeptide” is meant a protein with at least about 85% amino acid sequence identity to GenBank Accession No. AAA60082.1, which is provided below, or a fragment thereof. A functional PAH polypeptide is capable of catalyzing the hydroxylation of phenylalanine to tyrosine.

>AAA60082.1 Phenylalanine Hydroxylase [Homo sapiens]

(SEQ ID NO: 647)
MSTAVLENPGLGRKLSDFGQETSYIEDNCNQNGAISLIFSLKEEV
GALAKVLRLFEENDVNLTHIESRPSRLKKDEYEFFTHLDKRSLPA
LTNIIKILRHDIGATVHELSRDKKKDTVPWFPRTIQELDRFANQI
LSYGAELDADHPGFKDPVYRARRKQFADIAYNYRHGQPIPRVEYM
EEEKKTWGTVFKTLKSLYKTHACYEYNHIFPLLEKYCGFHEDNIP
QLEDVSQFLQTCTGFRLRPVAGLLSSRDFLGGLAFRVFHCTQYIR
HGSKPMYTPEPDICHELLGHVPLFSDRSFAQFSQEIGLASLGAPD
EYIEKLATIYWFTVEFGLCKQGDSIKAYGAGLLSSFGELQYCLSE
KPKLLPLELEKTAIQNYTVTEFQPLYYVAESFNDAKEKVRNFAAT
IPRPFSVRYDPYTQRIEVLDNTQQLKILADSINSEIGILCSALQK
IK

By “phenylalanine hydroxylase (PAH) polynucleotide” is meant a nucleic acid molecule encoding a PAH polypeptide, as well as the introns, exons, 3′ untranslated regions, 5′ untranslated regions, and regulatory sequences associated with its expression, or fragments thereof. In embodiments, a PAH polynucleotide is the genomic sequence, cDNA, mRNA, or gene associated with and/or required for PAH expression. An exemplary PAH polynucleotide sequence is provided at ENSEMBL Gene Accession No. ENSG00000171759 (SEQ ID NO: 649). A further exemplary PAH nucleotide sequence from Homo Sapiens is provided below (GenBank Accession No.: K03020.1):

>K03020.1:223-1581 Human Phenylalanine Hydroxylase mRNA, Complete Cds

(SEQ ID NO: 648)
ATGTCCACTGCGGTCCTGGAAAACCCAGGCTTGGGCAGGAAACTC
TCTGACTTTGGACAGGAAACAAGCTATATTGAAGACAACTGCAAT
CAAAATGGTGCCATATCACTGATCTTCTCACTCAAAGAAGAAGTT
GGTGCATTGGCCAAAGTATTGCGCTTATTTGAGGAGAATGATGTA
AACCTGACCCACATTGAATCTAGACCTTCTCGTTTAAAGAAAGAT
GAGTATGAATTTTTCACCCATTTGGATAAACGTAGCCTGCCTGCT
CTGACAAACATCATCAAGATCTTGAGGCATGACATTGGTGCCACT
GTCCATGAGCTTTCACGAGATAAGAAGAAAGACACAGTGCCCTGG
TTCCCAAGAACCATTCAAGAGCTGGACAGATTTGCCAATCAGATT
CTCAGCTATGGAGCGGAACTGGATGCTGACCACCCTGGTTTTAAA
GATCCTGTGTACCGTGCAAGACGGAAGCAGTTTGCTGACATTGCC
TACAACTACCGCCATGGGCAGCCCATCCCTCGAGTGGAATACATG
GAGGAAGAAAAGAAAACATGGGGCACAGTGTTCAAGACTCTGAAG
TCCTTGTATAAAACCCATGCTTGCTATGAGTACAATCACATTTTT
CCACTTCTTGAAAAGTACTGTGGCTTCCATGAAGATAACATTCCC
CAGCTGGAAGACGTTTCTCAATTCCTGCAGACTTGCACTGGTTTC
CGCCTCCGACCTGTGGCTGGCCTGCTTTCCTCTCGGGATTTCTTG
GGTGGCCTGGCCTTCCGAGTCTTCCACTGCACACAGTACATCAGA
CATGGATCCAAGCCCATGTATACCCCCGAACCTGACATCTGCCAT
GAGCTGTTGGGACATGTGCCCTTGTTTTCAGATCGCAGCTTTGCC
CAGTTTTCCCAGGAAATTGGCCTTGCCTCTCTGGGTGCACCTGAT
GAATACATTGAAAAGCTCGCCACAATTTACTGGTTTACTGTGGAG
TTTGGGCTCTGCAAACAAGGAGACTCCATAAAGGCATATGGTGCT
GGGCTCCTGTCATCCTTTGGTGAATTACAGTACTGCTTATCAGAG
AAGCCAAAGCTTCTCCCCCTGGAGCTGGAGAAGACAGCCATCCAA
AATTACACTGTCACGGAGTTCCAGCCCCTGTATTACGTGGCAGAG
AGTTTTAATGATGCCAAGGAGAAAGTAAGGAACTTTGCTGCCACA
ATACCTCGGCCCTTCTCAGTTCGCTACGACCCATACACCCAAAGG
ATTGAGGTCTTGGACAATACCCAGCAGCTTAAGATTTTGGCTGAT
TCCATTAACAGTGAAATTGGAATCCTTTGCAGTGCCCTCCAGAAA
ATAAAGTAA

By “adenine” or “9H-Purin-6-amine” is meant a purine nucleobase with the molecular formula C₅H₅N₅, having the structure

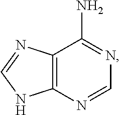

and corresponding to CAS No. 73-24-5.

By “adenosine” or “4-Amino-1-[(2R,3R,4S,5R)-3,4-dihydroxy-5-(hydroxymethyl)oxolan-2-yl]pyrimidin-2(1H)-one” is meant an adenine molecule attached to a ribose sugar via a glycosidic bond, having the structure

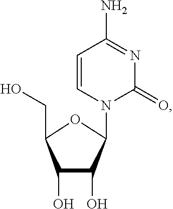

and corresponding to CAS No. 65-46-3. Its molecular formula is C₁₀H₁₃N₅O₄.

By “adenosine deaminase” or “adenine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism (e.g., eukaryotic, prokaryotic), including but not limited to algae, bacteria, fungi, plants, invertebrates (e.g., insects), and vertebrates (e.g., amphibians, mammals). In some embodiments, the adenosine deaminase is an adenosine deaminase variant with one or more alterations and is capable of deaminating both adenine and cytosine in a target polynucleotide (e.g., DNA, RNA) and may be referred to as a “dual deaminase”. Non-limiting examples of dual deaminases include those described in PCT/US22/22050. In some embodiments, the target polynucleotide is single or double stranded. In some embodiments, the adenosine deaminase variant is capable of deaminating both adenine and cytosine in DNA. In some embodiments, the adenosine deaminase variant is capable of deaminating both adenine and cytosine in single-stranded DNA. In some embodiments, the adenosine deaminase variant is capable of deaminating both adenine and cytosine in RNA. In embodiments, the adenosine deaminase variant is selected from those described in PCT/US2020/018192, PCT/US2020/049975, PCT/US2017/045381, PCT/US2021/016827, PCT/US2022/073781, PCT/US24/34189, or PCT/US2020/028568, the full contents of which are each incorporated herein by reference in their entireties for all purposes. Further non-limiting examples of adenosine deaminases include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. Further exemplary adenosine deaminase amino acid sequences include: TadA-8e (SEQ ID NO: 470), Tad1 (SEQ ID NO: 471), Tad2 (SEQ ID NO: 472), Tad3 (SEQ ID NO: 473), Tad4 (SEQ ID NO: 474), Tad6 (SEQ ID NO: 475), Tad6-SR (SEQ ID NO: 476), TadA9 (SEQ ID NO: 477), TadA20 (SEQ ID NO: 478), Staphylococcus aureus TadA (SEQ ID NO: 479), Bacillus subtilis TadA (SEQ ID NO: 480), Salmonella typhimurium TadA (SEQ ID NO: 481), Shewanella putrefaciens (SEQ ID NO: 482), Haemophilus influenzae F3031 TadA (SEQ ID NO: 483), Caulobacter crescentus TadA (SEQ ID NO: 484), Geobacter sulfurreducens TadA (SEQ ID NO: 485), Streptococcus pyogenes TadA (SEQ ID NO: 486), Aquifex aeolicus TadA (SEQ ID NO: 487), and E. coli TadA deaminase (ecTadA) (SEQ ID NO: 488).

By “adenosine deaminase activity” is meant catalyzing the deamination of adenine or adenosine to guanine in a polynucleotide.

By “Adenosine Base Editor (ABE)” is meant a base editor comprising an adenosine deaminase.

By “Adenosine Base Editor (ABE) polynucleotide” is meant a polynucleotide encoding an ABE.

By “Adenosine Base Editor 8 (ABE8) polypeptide” or “ABE8” is meant a base editor as defined herein comprising an adenosine deaminase or adenosine deaminase variant comprising one or more of the alterations listed in Table 5B, one of the combinations of alterations listed in Table 5B, or an alteration at one or more of the amino acid positions listed in Table 5B, where such alterations are relative to the following reference sequence: MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a corresponding position in another adenosine deaminase. In embodiments, ABE8 comprises alterations at amino acids 82 and/or 166 of SEQ ID NO: 1. In some embodiments, ABE8 comprises further alterations, as described herein, relative to the reference sequence.

By “Adenosine Base Editor 8 (ABE8) polynucleotide” is meant a polynucleotide encoding an ABE8 polypeptide.

“Administering” is referred to herein as providing one or more compositions described herein to a patient or a subject. By way of example and without limitation, composition administration (e.g., injection) can be performed by intravenous (i.v.) injection, subcutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally. Alternatively, or concurrently, administration can be by the oral route.

By “agent” is meant any small molecule chemical compound, antibody, nucleic acid molecule, polypeptide, or fragments thereof. In an embodiment, the agent is a base editor system described herein or a component thereof.

By “alteration” is meant a change in the level, structure, or activity of an analyte, gene or polypeptide as detected by standard art known methods such as those described herein. As used herein, an alteration includes a change (e.g., increase or reduction) in expression levels. In embodiments, the increase or reduction in expression levels is by 10%, 25%, 40%, 50% or greater. In some embodiments, an alteration includes an insertion, deletion, or substitution of a nucleobase or amino acid (by, e.g., genetic engineering).

By “ameliorate” is meant reduce, suppress, attenuate, diminish, arrest, or stabilize the development or progression of a disease. In embodiments, the disease is phenylketonuria (PKU), an autosomal recessive inborn error of metabolism associated with a deficiency of PAH. Editing of a PAH gene in a subject having PKU may ameliorate symptoms associated with the disease.

By “analog” is meant a molecule that is not identical but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor polypeptide (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain (e.g., Cas9 or Cpf1). Representative nucleic acid and protein sequences of base editors include those sequences having about or at least about 85% sequence identity to any base editor sequence provided in the sequence listing, such as those corresponding to SEQ ID NOs: 2-11.

By “BE4 cytidine deaminase (BE4) polypeptide,” is meant a base editor comprising a nucleic acid programmable DNA binding protein (napDNAbp) domain, a cytidine deaminase domain, and two uracil glycosylase inhibitor domains (UGIs). In embodiments, the napDNAbp is a Cas9n (D10A) polypeptide. Non-limiting examples of cytidine deaminase domains include rAPOBEC, ppAPOBEC, RrA3F, AmAPOBEC1, and SsAPOBEC3B.

By “BE4 cytidine deaminase (BE4) polynucleotide,” is meant a polynucleotide encoding a BE4 polypeptide.

By “base editing activity” is meant acting to chemically alter a base within a polynucleotide. In one embodiment, a first base is converted to a second base. In one embodiment, the base editing activity is cytidine deaminase activity, e.g., converting target C⋅G to T⋅A. In another embodiment, the base editing activity is adenosine or adenine deaminase activity, e.g., converting A⋅T to G⋅C.

The term “base editor system” refers to an intermolecular complex for editing a nucleobase of a target nucleotide sequence. In various embodiments, the base editor (BE) system comprises (1) a polynucleotide programmable nucleotide binding domain, a deaminase domain (e.g., cytidine deaminase or adenosine deaminase) for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domain selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine or cytosine base editor (CBE). In some embodiments, the base editor system (e.g., a base editor system comprising a cytidine deaminase) comprises a uracil glycosylase inhibitor or other agent or peptide (e.g., a uracil stabilizing protein such as provided in WO2022015969, the disclosure of which is incorporated herein by reference in its entirety for all purposes) that inhibits the inosine base excision repair system.

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease.

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

Amino acids generally can be grouped into classes according to the following common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

In some embodiments, conservative substitutions can involve the exchange of a member of one of these classes for another member of the same class. In some embodiments, non-conservative amino acid substitutions can involve exchanging a member of one of these classes for another class.

The term “coding sequence” or “protein coding sequence” as used interchangeably herein refers to a segment of a polynucleotide that codes for a protein. Coding sequences can also be referred to as open reading frames. The region or sequence is bounded nearer the 5′ end by a start codon and nearer the 3′ end with a stop codon. Stop codons useful with the base editors described herein include the following: TAG, TAA, and TGA.

By “complex” is meant a combination of two or more molecules whose interaction relies on inter-molecular forces. Non-limiting examples of inter-molecular forces include covalent and non-covalent interactions. Non-limiting examples of non-covalent interactions include hydrogen bonding, ionic bonding, halogen bonding, hydrophobic bonding, van der Waals interactions (e.g., dipole-dipole interactions, dipole-induced dipole interactions, and London dispersion forces), and π-effects. In an embodiment, a complex comprises polypeptides, polynucleotides, or a combination of one or more polypeptides and one or more polynucleotides. In one embodiment, a complex comprises one or more polypeptides that associate to form a base editor (e.g., base editor comprising a nucleic acid programmable DNA binding protein, such as Cas9, and a deaminase) and a polynucleotide (e.g., a guide RNA). In an embodiment, the complex is held together by hydrogen bonds. It should be appreciated that one or more components of a base editor (e.g., a deaminase, or a nucleic acid programmable DNA binding protein) may associate covalently or non-covalently. As one example, a base editor may include a deaminase covalently linked to a nucleic acid programmable DNA binding protein (e.g., by a peptide bond). Alternatively, a base editor may include a deaminase and a nucleic acid programmable DNA binding protein that associate noncovalently (e.g., where one or more components of the base editor are supplied in trans and associate directly or via another molecule such as a protein or nucleic acid). In an embodiment, one or more components of the complex are held together by hydrogen bonds.

By “cytosine” or “4-Aminopyrimidin-2(1H)-one” is meant a purine nucleobase with the molecular formula C₄H₅N₃O, having the structure

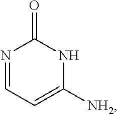

and corresponding to CAS No. 71-30-7.

By “cytidine” is meant a cytosine molecule attached to a ribose sugar via a glycosidic bond, having the structure

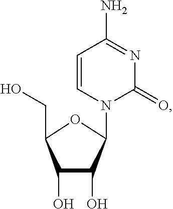

and corresponding to CAS No. 65-46-3. Its molecular formula is C₉H₁₃N₃O₅.

By “Cytidine Base Editor (CBE)” is meant a base editor comprising a cytidine deaminase. Non-limiting examples of cytidine deaminase base editor amino acid sequences include amino acid sequences for BE4max (SEQ ID NO: 553), YE1-BE4 (SEQ ID NO: 554), YE2-BE4 (SEQ ID NO: 555), YEE-BE4 (SEQ ID NO: 556), EE-BE4 (SEQ ID NO: 557), R33A−BE4 (SEQ ID NO: 558), R33A+K34A-BE4 (SEQ ID NO: 559), APOBEC3A (A3A)-BE4 (SEQ ID NO: 560), APOBEC3B (A3B)-BE4 (SEQ ID NO: 561), APOBEC3G (A3G)-BE4 (SEQ ID NO: 562), AID-BE4 (SEQ ID NO: 563), CDA-BE4 (SEQ ID NO: 564), FERNY-BE4 (SEQ ID NO: 565), evolved APOBEC3A (eA3A)-BE4 (SEQ ID NO: 566), AALN-BE4 (SEQ ID NO: 567), BE4max modified with SpCas9-NG (SEQ ID NO: 568), YE1-SpCas9-NG (YE1-NG) (SEQ ID NO: 569), YE2-SpCas9-NG (SEQ ID NO: 570), YEE-SpCas9-NG (SEQ ID NO: 571), EE-SpCas9-NG (SEQ ID NO: 572), R33A+K34A-SpCas9-NG (SEQ ID NO: 573), YE1-CP1028 (YE1-BE4-CP1028, or YE1-CP) (SEQ ID NO: 574), YE2-CP1028 (YE2-BE4-CP1028) (SEQ ID NO: 575), YEE-CP1028 (YEE-BE4-CP1028) (SEQ ID NO: 576), EE-CP1028 (EE-BE4-CP1028) (SEQ ID NO: 577), R33A+K34A-CP1028 (R33A+K34A-BE4-CP1028) (SEQ ID NO: 578), BE4max (with nickase) (SEQ ID NO: 597), BE4 (SEQ ID NO: 598), BE4 with His tag (SEQ ID NO: 599), BE4max (SEQ ID NO: 600), AncBE4max 689 (SEQ ID NO: 601), and AncBE4max 687 (SEQ ID NO: 602).

By “Cytidine Base Editor (CBE) polynucleotide” is meant a polynucleotide encoding a CBE. Non-limiting examples of polynucleotide sequences encoding cytidine deaminase base editors include those encoding BE4max (SEQ ID NO: 616), AncBE4max689 (SEQ ID NO: 617), and AncBE4max687 (SEQ ID NO: 618).

By “cytidine deaminase” or “cytosine deaminase” is meant a polypeptide or fragment thereof capable of deaminating cytidine or cytosine. In embodiments, the cytidine or cytosine is present in a polynucleotide. In one embodiment, the cytidine deaminase converts cytosine to uracil or 5-methylcytosine to thymine. The terms “cytidine deaminase” and “cytosine deaminase” are used interchangeably throughout the application. Petromyzon marinus cytosine deaminase 1 (PmCDA1) (SEQ ID NO: 13-14), Activation-induced cytidine deaminase (AICDA) (SEQ ID NOs: 15-21), and APOBEC (SEQ ID NOs: 12-61) are exemplary cytidine deaminases. Further exemplary cytidine deaminase (CDA) sequences are provided in the Sequence Listing as SEQ ID NOs: 62-66 and SEQ ID NOs: 67-189. Non-limiting examples of cytidine deaminases include those described in PCT/US20/16288, PCT/US2018/021878, 180802-021804/PCT, PCT/US2018/048969, PCT/US2016/058344, PCT/US2020/062428, and PCT/US2019/033848, the disclosures of which are incorporated herein by reference in their entireties for all purposes. Non-limiting examples of cytidine deaminase amino acid sequences include amino acid sequences for Rat APOBEC1 (SEQ ID NO: 579), Human APOBEC1 (SEQ ID NO: 580), Human APOBEC3 (SEQ ID NO: 581), Human APOBEC3B (SEQ ID NO: 582), Human APOBEC3G (SEQ ID NO: 583), evoAPOBEC3A (eA3A) (SEQ ID NO: 584), evoCDA (SEQ ID NO: 585), evoAPOBEC1 (SEQ ID NO: 586), YE1 (SEQ ID NO: 587), YE2 (SEQ ID NO: 588), YEE (SEQ ID NO: 589), EE (SEQ ID NO: 590), R33A (SEQ ID NO: 591), R33A+K34A (SEQ ID NO: 592), AALN (SEQ ID NO: 593), FERNY (SEQ ID NO: 594), evoFERNY (SEQ ID NO: 595), APOBEC (SEQ ID NO: 619), Anc686 APOBEC (SEQ ID NO: 620), Human APOBEC-3G D316R_D317R (SEQ ID NO: 621), Human APOBEC-3G chain A (SEQ ID NO: 622), Human APOBEC3-G chain A D120R_D121R (SEQ ID NO: 623), Mouse APOBEC3 (SEQ ID NO: 624), Rat APOBEC3 (SEQ ID NO: 625), Rhesus macaque APOBEC-3G (SEQ ID NO: 626), Chimpanzee APOBEC-3G (SEQ ID NO: 627), Green Monkey APOBEC-3G (SEQ ID NO: 628), Human APOBEC-3G (SEQ ID NO: 629), Human APOBEC-3F (SEQ ID NO: 630), Human APOBEC-3B (SEQ ID NO: 631), Rat APOBEC-3B (SEQ ID NO: 632), Bovine APOBEC-3B (SEQ ID NO: 633), Chimpanzee APOBEC-3B (SEQ ID NO: 634), Gorilla APOBEC-3C (SEQ ID NO: 635), Human APOBEC-3A (SEQ ID NO: 636), Rhesus macaque APOBEC-3A (SEQ ID NO: 637), Bovine APOBEC-3A (SEQ ID NO: 638), Human APOBEC-3H (SEQ ID NO: 639), Human APOBEC-3D (SEQ ID NO: 640), Rat ABOPEC1 (SEQ ID NO: 641), Anc689 APOBEC (SEQ ID NO: 642), Anc687 APOBEC (SEQ ID NO: 643), Anc686 APOBEC (SEQ ID NO: 644), Anc655 APOBEC (SEQ ID NO: 645), and Anc733 APOBEC (SEQ ID NO: 646).

By “cytidine deaminase polynucleotide” is meant a polynucleotide encoding a cytidine deaminase. Non-limiting examples of polynucleotide sequences encoding cytidine deaminase domains include those encoding Rat APOBEC1 (SEQ ID NO: 604), Anc689 APOBEC (SEQ ID NO: 605), Anc687 APOBEC (SEQ ID NO: 606), Anc686 APOBEC (SEQ ID NO: 607), Anc655 APOBEC (SEQ ID NO: 608), Anc733 APOBEC (SEQ ID NO: 609), Rat APOBEC1 (SEQ ID NO: 610), Anc689 APOBEC (SEQ ID NO: 611), Anc687 APOBEC (SEQ ID NO: 612), Anc686 APOBEC (SEQ ID NO: 613), Anc655 APOBEC (SEQ ID NO: 614), and Anc733 APOBEC (SEQ ID NO: 615).

By “cytosine deaminase activity” is meant catalyzing the deamination of cytosine or cytidine. In one embodiment, a polypeptide having cytosine deaminase activity converts an amino group to a carbonyl group. In an embodiment, a cytosine deaminase converts cytosine to uracil (i.e., C to U) or 5-methylcytosine to thymine (i.e., 5mC to T). In some embodiments, a cytosine deaminase as provided herein has increased cytosine deaminase activity (e.g., at least 10-fold, 20-fold, 30-fold, 40-fold, 50-fold, 60-fold, 70-fold, 80-fold, 90-fold, 100-fold or more) relative to a reference cytosine deaminase.

The term “deaminase” or “deaminase domain,” as used herein, refers to a protein or fragment thereof that catalyzes a deamination reaction.

The term, “detect” refers to identifying the presence, absence or amount of the analyte to be detected. In one embodiment, a sequence alteration in a polynucleotide or polypeptide is detected. In another embodiment, the presence of indels is detected. In one embodiment, PAH activity is detected. In another embodiment, levels of Phe in a biological sample (e.g., blood, serum, plasma, tissue) are detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA)), biotin, digoxigenin, or haptens.

By “disease” is meant any condition or disorder that damages or interferes with the normal function of a cell, tissue, or organ. Exemplary diseases include phenylketonuria.

By “dual editing activity” or “dual deaminase activity” is meant having adenosine deaminase and cytidine deaminase activity. In one embodiment, a base editor having dual editing activity has both A→G and C→T activity, wherein the two activities are approximately equal or are within about 10% or 20% of each other. In another embodiment, a dual editor has A→G activity that no more than about 10% or 20% greater than C→T activity. In another embodiment, a dual editor has A→G activity that is no more than about 10% or 20% less than C→T activity. In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. Non-limiting examples of proteins having dual deaminase activity include those described in International Patent Application Publications No. WO 2024/040083 and WO 2022/204574, the disclosures of which are hereby incorporated by reference in their entireties for all purposes.

By “effective amount” is meant the amount of an agent (e.g., a base editor, cell) as described herein, that is required to ameliorate the symptoms of a disease relative to an untreated patient or an individual without disease, i.e., a healthy individual, or is the amount of the agent sufficient to elicit a desired biological response. The effective amount of active compound(s) used to practice embodiments of the present disclosure for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the disclosure sufficient to introduce an alteration in a gene of interest in a cell (e.g., a cell in vitro or in vivo). In one embodiment, an effective amount is the amount of a base editor required to achieve a therapeutic effect. Such therapeutic effect need not be sufficient to alter a pathogenic gene in all cells of a subject, tissue or organ, but only to alter the pathogenic gene in about 1%, 5%, 10%, 25%, 50%, 75% or more of the cells present in a subject, tissue or organ. In one embodiment, an effective amount is sufficient to ameliorate one or more symptoms of a disease. For example, an effective amount of a base editor described herein is delivered to a tissue of a patient where it increases the level of PAH expression, thereby reducing the accumulation of Phe in the serum of the patient.

By “fragment” is meant a portion of a polypeptide or nucleic acid molecule. This portion contains, at least about 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids. In some embodiments, the fragment is a functional fragment.

By “guide polynucleotide” is meant a polynucleotide or polynucleotide complex which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

By “increase” is meant a positive alteration of at least 10%, 25%, 50%, 75%, or 100%, or about 1.5 fold, about 2 fold, about 3-fold, about 4-fold, about 5-fold, about 6-fold, about 7-fold, about 8-fold, about 9-fold, about 10-fold, about 15-fold, about 20-fold, about 25-fold, about 30-fold, about 35-fold, about 40-fold, about 45-fold, about 50-fold, or about 100-fold. In embodiments, expression of a base editor described herein increases expression of PAH in a tissue of a subject.

The terms “inhibitor of base repair”, “base repair inhibitor”, “IBR” or their grammatical equivalents refer to a protein that is capable in inhibiting the activity of a nucleic acid repair enzyme, for example a base excision repair enzyme.

An “intein” is a fragment of a protein that is able to excise itself and join the remaining fragments (the exteins) with a peptide bond in a process known as protein splicing.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this disclosure is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid molecule that is free of the genes which, in the naturally-occurring genome of the organism from which the nucleic acid molecule of the disclosure is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the disclosure that has been separated from components that naturally accompany it. Typically, the polypeptide is isolated when it is at least 60%, by weight, free from the proteins and naturally-occurring organic molecules with which it is naturally associated. In embodiments, the preparation is at least 75%, at least 90%, or at least 99%, by weight, a polypeptide of the disclosure. An isolated polypeptide of the disclosure may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, refers to a molecule that links two moieties. In one embodiment, the term “linker” refers to a covalent linker (e.g., covalent bond) or a non-covalent linker.

By “marker” is meant any protein or polynucleotide having an alteration in expression, level, structure, or activity that is associated with a disease or disorder. In an embodiment, the level of Phe in a sample (e.g., blood, serum, plasma, tissue) is a marker of PKU.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi: 10.1038/nbt.4172. In some embodiments, an NLS comprises the amino acid sequence

(SEQ ID NO: 190)
KRTADGSEFESPKKKRKV,
(SEQ ID NO: 191)
KRPAATKKAGQAKKKK,
(SEQ ID NO: 192)
KKTELQTTNAENKTKKL,
(SEQ ID NO: 193)
KRGINDRNFWRGENGRKTR,
(SEQ ID NO: 194)
RKSGKIAAIVVKRPRK,
(SEQ ID NO: 195)
PKKKRKV,
(SEQ ID NO: 196)
MDSLLMNRRKFLYQFKNVRWAKGRRETYLC,
(SEQ ID NO: 328)
PKKKRKVEGADKRTADGSEFESPKKKRKV,
or
(SEQ ID NO: 329)
RKSGKIAAIVVKRPRKPKKKRKV.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (I). A “nucleotide” consists of a nucleobase, a five carbon sugar (either ribose or deoxyribose), and at least one phosphate group. Non-limiting examples of modified nucleobases and/or chemical modifications that a modified nucleobase may include are the following: pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine.

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, Cas12i, and Cas12j/CasΦ (Cas12j/Casphi). Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Cas12j/CasΦ, Cpf1, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363 (6422): 88-91. doi: 10.1126/science.aav7271, the entire contents of each are hereby incorporated by reference. Exemplary nucleic acid programmable DNA binding proteins and nucleic acid sequences encoding nucleic acid programmable DNA binding proteins are provided in the Sequence Listing as SEQ ID NOs: 197-231, 232-245, 254-257, 260, and 378. In some embodiments, the napDNAbp is a (CRISPR-associated system) Cas9 endonuclease, for example, Cas9 (Csn1) from Streptococcus pyogenes (e.g., SEQ ID NO: 197), Cas9 from Neisseria meningitidis (NmeCas9; SEQ ID NO: 208), Nme2Cas9 (SEQ ID NO: 209), Streptococcus constellatus (ScoCas9), or derivatives thereof (e.g., a sequence with at least about 85% sequence identity to a Cas9, such as Nme2Cas9 or spCas9). Further non-limiting examples of nucleic acid programmable DNA binding proteins include those disclosed or referenced in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes, which were designed using artificial intelligence. In some embodiments, the napDNAbp is OpenCRISPR-1, or a variant thereof (e.g., a variant comprising a D10A amino acid alteration and/or lacking an N-terminal methionine). Further non-limiting examples of nucleic acid programmable DNA binding proteins include those disclosed in International Patent Application No. PCT/US2019/047996.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase).

By “OpenCRISPR-1 polypeptide” is meant a protein with an amino acid sequence having at least about 85% amino acid sequence identity to SEQ ID NO: 463, or a fragment thereof that associates with a nucleic acid, such as a guide nucleic acid or guide polynucleotide, that guides the napDNAbp to a specific nucleic acid sequence. Further details relating to the OpenCRISPR-1 polypeptide are disclosed in Rufflow, et al., “Design of highly functional genome editors by modeling of the universe of CRISPR-Cas Sequences,” bioRxiv, posted Apr. 22, 2024, doi: 10.1101/2024.04.22.590591, the disclosure of which is incorporated herein by reference in its entirety for all purposes.

By “OpenCRISPR-1 polynucleotide” is meant a nucleic acid molecule encoding an OpenCRISPR-1 polypeptide, as well as the introns, exons, 3′ untranslated regions, 5′ untranslated regions, and regulatory sequences associated with its expression, or fragments thereof. In embodiments, an OpenCRISPR-1 polynucleotide is the genomic sequence, cDNA, mRNA, or gene associated with and/or required for OpenCRISPR-1 expression. An exemplary OpenCRISPR-1 nucleotide sequence is provided at SEQ ID NO: 464.

In various embodiments, a guide RNA suitable for use in combination with an OpenCRISPR-1 polypeptide contains a scaffold having at least 85% sequence identity to a nucleotide sequence selected from the following, or fragments thereof capable of binding to an OpenCRISPR-1 polypeptide:

(SEQ ID NO: 465)
GUUUUAGAGCUGUGUUGAAAAACACAGCAAGUUAAAAUAAGGCUU
UGUCCGUAUCCAACUUGAAAAAGUGAGCACCGAUUCGGUGC;
(SEQ ID NO: 466)
GUUUUAGAGCUGGAAACAGCAAGUUAAAAUAAGGCUUUGUCCGUA
UCCAACUUGAAAAAGUGAGCACCGAUUCGGUGC;
and
(SEQ ID NO: 467)
GUUUUAGAGCUAGAAAUAGCAAGUUAAAAUAAGGCUAGUCCGUUA
UCAACUUGAAAAAGUGGCACCGAGUCGGUGC

As used herein, “obtaining” as in “obtaining an agent” includes synthesizing, purchasing, or otherwise acquiring the agent.

By “subject” or “patient” is meant a mammal, including, but not limited to, a human or non-human mammal. In embodiments, the mammal is a bovine, equine, canine, ovine, rabbit, rodent, nonhuman primate, or feline. In an embodiment, “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “pathogenic mutation”, “pathogenic variant”, “disease causing mutation”, “disease causing variant”, “deleterious mutation”, or “predisposing mutation” refers to a genetic alteration or mutation that is associated with a disease or disorder or that increases an individual's susceptibility or predisposition to a certain disease or disorder. In some embodiments, the pathogenic mutation comprises at least one wild-type amino acid substituted by at least one pathogenic amino acid in a protein encoded by a gene. In some embodiments, the pathogenic mutation is in a terminating region (e.g., stop codon). In some embodiments, the pathogenic mutation is in a non-coding region (e.g., intron, promoter, etc.).

The terms “protein”, “peptide”, “polypeptide”, and their grammatical equivalents are used interchangeably herein, and refer to a polymer of amino acid residues linked together by peptide (amide) bonds. A protein, peptide, or polypeptide can be naturally occurring, recombinant, or synthetic, or any combination thereof.

The term “fusion protein” as used herein refers to a hybrid polypeptide which comprises protein domains from at least two different proteins.

The term “recombinant” as used herein in the context of proteins or nucleic acids refers to proteins or nucleic acids that do not occur in nature but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%. In embodiments, a base editor system of the disclosure reduces and/or normalizes levels of Phe in a biological sample of the subject.

By “reference” is meant a standard or control condition. In one embodiment, the reference is the level of Phe present in the blood of a subject not having PKU. In another embodiment, the reference is the level of Phe present in the blood of a subject having PKU prior to treatment. In another embodiment, the reference is a number that describes an ideal range for Phe levels in blood. In other embodiments and without limitation, a reference is an untreated cell that is not subjected to a test condition, or is subjected to placebo or normal saline, medium, buffer, and/or a control vector that does not harbor a polynucleotide of interest. In embodiments, a reference is a base editor system containing a base editor polypeptide and/or guide polynucleotide that differs from that of a base editor system of interest.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” refer to a nuclease that forms a complex with (e.g., binds or associates with) one or more RNA(s) that is not a target for cleavage. In some embodiments, an RNA-programmable nuclease, when in a complex with an RNA, may be referred to as a nuclease-RNA complex. Typically, the bound RNA(s) is referred to as a guide RNA (gRNA).

The term “single nucleotide polymorphism (SNP)” refers to a variation in a single nucleotide that occurs at a specific position in the genome, where each variation is present to some appreciable degree within a population (e.g., >1%). SNPs can fall within coding regions of genes, non-coding regions of genes, or in the intergenic regions (regions between genes). In some embodiments, SNPs within a coding sequence do not necessarily change the amino acid sequence of the protein that is produced, due to degeneracy of the genetic code. SNPs in the coding region are of two types: synonymous and nonsynonymous SNPs. Synonymous SNPs do not affect the protein sequence, while nonsynonymous SNPs change the amino acid sequence of protein. The nonsynonymous SNPs are of two types: missense and nonsense. SNPs that are not in protein-coding regions can still affect gene splicing, transcription factor binding, messenger RNA degradation, or the sequence of noncoding RNA. Gene expression affected by this type of SNP is referred to as an eSNP (expression SNP) and can be upstream or downstream from the gene. A single nucleotide variant (SNV) is a variation in a single nucleotide without any limitations of frequency and can arise in somatic cells. A somatic single nucleotide variation can also be called a single-nucleotide alteration.

By “specifically binds” is meant a nucleic acid molecule, polypeptide, polypeptide/polynucleotide complex, compound, or molecule that recognizes and binds a polypeptide and/or nucleic acid molecule of the disclosure, but which does not substantially recognize and bind other molecules in a sample, for example, a biological sample.

By “substantially identical” is meant a polypeptide or nucleic acid molecule exhibiting at least 50% identity to a reference amino acid sequence. In one embodiment, a reference sequence is a wild-type amino acid or nucleic acid sequence. In another embodiment, a reference sequence is any one of the amino acid or nucleic acid sequences described herein. In one embodiment, such a sequence is at least about 60%, 80%, 85%, 90%, 95%, 96%, 97%, 98%, 99%, 99.9%, or even 99.99%, identical at the amino acid level or nucleic acid level to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine.

Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the disclosure include any nucleic acid molecule that encodes a polypeptide of the disclosure or a functional fragment thereof. Such nucleic acid molecules need not be 100% identical with an endogenous nucleic acid sequence but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

By “split” is meant divided into two or more fragments.

A “split polypeptide” or “split protein” refers to a protein that is provided as an N-terminal fragment and a C-terminal fragment translated as two separate polypeptides from a nucleotide sequence(s). The polypeptides corresponding to the N-terminal portion and the C-terminal portion of the split protein may be spliced in some embodiments to form a “reconstituted” protein. In embodiments, the split polypeptide is a nucleic acid programmable DNA binding protein (e.g. a Cas9) or a base editor.

The term “target site” refers to a nucleotide sequence or nucleobase of interest within a nucleic acid molecule that is modified. In embodiments, the modification is deamination of a base. The deaminase can be a cytidine or an adenine deaminase. The fusion protein or base editing complex comprising a deaminase may comprise a dCas9-adenosine deaminase fusion protein, a Cas 12b-adenosine deaminase fusion, or a base editor disclosed herein.

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, reduces the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a composition as described herein.

By “uracil glycosylase inhibitor” or “UGI” is meant an agent that inhibits the uracil-excision repair system. Base editors comprising a cytidine deaminase convert cytosine to uracil, which is then converted to thymine through DNA replication or repair. In various embodiments, a uracil DNA glycosylase (UGI) prevent base excision repair which changes the U back to a C. In some instances, contacting a cell and/or polynucleotide with a UGI and a base editor prevents base excision repair which changes the U back to a C. An exemplary UGI comprises an amino acid sequence as follows:

>splP14739IUNGI_BPPB2 Uracil-DNA Glycosylase Inhibitor

(SEQ ID NO: 231)
MTNLSDIIEKETGKQLVIQESILMLPEEVEEVIGNKPESDILVHT
AYDESTDENVMLLTSDAPEYKPWALVIQDSNGENKIKML.

In some embodiments, the agent inhibiting the uracil-excision repair system is a uracil stabilizing protein (USP). See, e.g., WO 2022015969 A1, incorporated herein by reference.

As used herein, the term “vector” refers to a means of introducing a nucleic acid molecule into a cell, resulting in a transformed cell. Vectors include plasmids, transposons, phages, viruses, liposomes, lipid nanoparticles, and episomes.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

All terms are intended to be understood as they would be understood by a person skilled in the art. Unless defined otherwise, all technical and scientific terms used herein have the same meaning as commonly understood by one of ordinary skill in the art to which the disclosure pertains.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended. This wording indicates that specified elements, features, components, and/or method steps are present, but does not exclude the presence of other elements, features, components, and/or method steps. Any embodiments specified as “comprising” a particular component(s) or element(s) are also contemplated as “consisting of” or “consisting essentially of” the particular component(s) or element(s) in some embodiments. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

### BRIEF DESCRIPTION OF THE DRAWINGS

FIG. 1 provides a bar graph showing maximum percent A to G base editing of a phenylalanine hydroxylase (PAH) polynucleotide in GM02406 cells using the indicated base editor systems. In FIG. 1, each base editor system is identified using the following terminology: the identifier preceding the first underscore (“_”) indicates the guide polynucleotide (e.g., g5847), the identifier following the first underscore indicates the MRNA molecule encoding the base editor polypeptide (e.g., MRNA4018), the term “PAH” indicates the target gene, and the identifier following the last underscore indicates the protospacer adjacent motif bound by the nucleic acid programmable DNA binding protein (napDNAbp) of the base editor polypeptide. In FIG. 1, the base editor system “sg23_MRNA4247_ALAS1,” which targets an ALAS1 polynucleotide for base editing, was used as a positive control for base editing. The cells were transfected with 1000 ng of mRNA encoding the base editor and 333 ng of the guide polynucleotide (i.e., gRNA). Base editing was measured using next-generation sequencing 72 hours following transfection. 6×10e4 cells were used in each transfection.

FIGS. 2A-2E provide schematic diagrams and bar graphs. FIG. 2A provides a schematic diagram showing the PAH target site (boxed region) for the guide polynucleotide g5847. FIG. 2B provides a schematic diagram showing the PAH target site (boxed region) for the guide polynucleotide g5849. FIG. 2C provides a schematic diagram showing the PAH target site (boxed region) for the guide polynucleotide g5851. FIG. 2D provides a schematic diagram showing the PAH target site (boxed region) for the guide polynucleotide g5855. In each of FIGS. 2A-2D, the target nucleobase is shown as a lowercase “a,” and the amino acid sequence encoded by each depicted PAH sequence is provided beneath the same. FIG. 2E provides a series of bar graphs showing percent on-target (“correct allele”; left bars) and nonsynonymous bystander editing (right bars) A to G editing measured in GM02406 cells administered the indicated base editors. The sequences shown in FIGS. 2A-2E, in order of occurrence correspond to SEQ ID NOs: 655, 657, and 656. The guide polynucleotides targeted a base editor to deaminate a nucleotide corresponding to an R408W pathogenic mutation. In FIG. 2E, each base editor system is identified using the following terminology: the identifier preceding the first underscore (“_”) indicates the guide polynucleotide (e.g., g5847), the identifier following the first underscore indicates the MRNA molecule encoding the base editor polypeptide (e.g., MRNA2743), the term “PAH” indicates the target gene, and the identifier following the last underscore indicates the protospacer adjacent motif bound by the nucleic acid programmable DNA binding protein (napDNAbp) of the base editor polypeptide.

FIG. 3 provides a bar graph showing maximum total percent A to G base editing (y-axis) of a phenylalanine hydroxylase (PAH) polynucleotide in GM02406 cells using the indicated base editor systems. In FIG. 3, each base editor system is identified using the following terminology: the guide polynucleotide relating to each cluster of bars is identified above the same; the identifier preceding the first underscore (“_”) indicates the MRNA molecule encoding the base editor polypeptide (e.g., MRNA4247), the term “ABE” indicates an adenosine deaminase, the term following the term “ABE_” indicates the identity of the adenosine deaminase domain of the base editor polypeptide (e.g., 8.20 refers to TadA*8.20), “SpCas9” indicates that the base editor polypeptide contained an SpCas9 napDNAbp, and the identifier following the last underscore indicates the protospacer adjacent motif bound by the nucleic acid programmable DNA binding protein (napDNAbp) of the base editor polypeptide. In FIG. 1, the base editor system “sg23_MRNA4247_ALAS1,” which targets an ALAS1 polynucleotide for base editing, was used as a positive control for base editing. The cells were transfected with 1000 ng of mRNA encoding the base editor and 333 ng of the guide polynucleotide (i.e., gRNA). Base editing was measured using next-generation sequencing 72 hours following transfection. 6×10e4 cells were used in each transfection.

FIG. 4 provides a series of bar graphs showing percent on-target (“correct allele”; left bars) and nonsynonymous bystander editing (right bars) A to G editing measured in GM02406 cells administered the indicated base editors. In FIG. 4, each base editor system is identified using the following terminology: the identifier preceding the first underscore (“_”) indicates the guide polynucleotide (e.g., g5851), the identifier following the first underscore indicates the MRNA molecule encoding the base editor polypeptide (e.g., MRNA3170), and the identifier following the last underscore indicates the protospacer adjacent motif bound by the nucleic acid programmable DNA binding protein (napDNAbp) of the base editor polypeptide.

FIG. 5 provides a bar graph showing percent A to G base editing measured in untreated control GM02406 cells. The cells were not transfected with any mRNA or guide RNA. These data were helpful to empirically establish the baseline, rather than use the assumed 50% that might be expected for these cells derived from a heterozygous patient.

FIG. 6 provides a plot showing an editing profile or an ABE8.20 base editor, where positions are indicated relative to the protospacer adjacent motif (PAM), where the nucleobase adjacent to the PAM is position 1. The “nom_weight” indicated for each position is proportional to the probability that the position will be edited by ABE8.20 (e.g., position 5 is the most likely to be edited). In FIG. 6 the terms 5A, 5C, 5G, etc. indicate the “nom_weight” for base editing of the indicated base at the indicated particular position.

FIG. 7 provides a series of bar graphs showing percent on-target (“correct allele”; left bars) and nonsynonymous bystander editing (right bars) A to G editing measured in GM02406 cells administered the indicated base editors. In FIG. 7, each base editor system is identified using the following terminology: the identifier preceding the first underscore (“_”) indicates the guide polynucleotide (e.g., g5851), the identifier following the first underscore indicates the MRNA molecule encoding the base editor polypeptide (e.g., MRNA3170), and the identifier following the last underscore indicates the protospacer adjacent motif bound by the nucleic acid programmable DNA binding protein (napDNAbp) of the base editor polypeptide. Beneath each plot of FIG. 7 is provided a sequence for the target site corresponding to the data of the plot, where the nucleotide targeted for base editing is underlined and the PAM sequence is shown in bold. The nucleotide sequences provided beneath each plot correspond to SEQ ID NOs: 658-661, in order of occurrence. In FIG. 7 the term “ABE_8.20_SpCas9_NG” indicates an adenosine deaminase base editor (ABE) containing a TadA*8.20 adenosine deaminase domain and an SpCas9 napDNAbp domain with specificity for an NG PAM sequence, “ABE_8.20-IBE16_SpCas9_NGC” indicates an adenosine deaminase base editor (ABE) containing a TadA*8.20 adenosine deaminase domain inserted within an SpCas9 napDNAbp domain with specificity for an NGC PAM sequence.

### DETAILED DESCRIPTION

Provided herein are base editors, endonucleases, and guide RNAs (gRNAs) for use in editing, modifying, or altering a target polynucleotide. In particular embodiments, a base editor or endonuclease of the present disclosure modifies an phenylalanine hydroxylase (PAH) polynucleotide. In particular embodiments, a base editor of the disclosure deaminates a pathogenic nucleobase in a PAH polynucleotide to increase or restore activity of the phenylalanine hydroxylase polypeptide encoded by the same.

The various aspects and embodiments of the disclosure are based, at least in part, upon the discovery that base editing (e.g., to alter or correct a pathogenic nucleotide) can be used to increase the expression or activity of a PAH polypeptide, where a deficiency in PAH is associated with phenylketonuria (PKU). In particular, increasing expression and/or activity of the PAH polypeptide in a subject diagnosed with or having a propensity to develop phenylketonuria can be an effective treatment strategy. This increase in expression and/or activity can be effected using any of the base editing systems provided herein. Accordingly, the disclosure features compositions and methods for editing a PAH polynucleotide. The edit to the PAH polynucleotide is associated with an increase or restoration of expression or activity of a PAH polypeptide in a cell of a subject, as well as a decrease in Phe in the blood of the subject and/or a decrease in symptoms associated with PKU.

In embodiments, the methods of the present disclosure include altering a PAH polynucleotide to restore or increase activity of a PAH polypeptide encoded thereby. For example, the base editors or base editor systems provided herein can be used for editing a nucleobase in an exon of the PAH polynucleotide. In some embodiments, the target sequence is or contains codon 408 of the PAH polynucleotide. In some embodiments, the deamination of an A or C nucleobase in the exon results in a restoration or increase of activity of a PAH polypeptide. In some embodiments, the subject has or has the potential to develop phenylketonuria.

In some instances, the methods of the present disclosure include modifying a PAH polynucleotide to increase levels or activity of the PAH polynucleotide and/or polypeptide. The alterations can be effected by a base editor system, such as those described herein.

In some embodiments, the present disclosure provides base editor systems that efficiently generate an intended mutation, such as an alteration of a nucleotide, in a nucleic acid molecule (e.g., a nucleic acid within a genome of a subject) without generating a significant number of unintended mutations, such as unintended point mutations. In some embodiments, an intended mutation is a mutation that is generated by a specific base editor (e.g., an adenosine base editor or a cytidine base editor) bound to a guide polynucleotide (e.g., gRNA) specifically designed to generate the intended mutation. In some embodiments, the intended mutation is an adenine (A) to guanine (G) point mutation within the coding region of a gene. In some embodiments, the intended mutation is a cytosine (C) to thymine (T) point mutation within the coding region of a gene. In some embodiments, the intended mutation is a mutation of a nucleobase corresponding to codon 408 of a PAH polynucleotide, where in some embodiments codon 408 encodes a tryptophan (W) amino acid prior to base editing. In some instances, the intended mutation results in a W408R amino acid alteration in the PAH polypeptide encoded by the PAH polynucleotide. In some embodiments, the intended mutation is an adenine (A) to guanine (G) point mutation in codon 408 of the PAH polynucleotide. In some embodiments, the intended mutation is a missense mutation.

In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more.

In some embodiments, editing of a plurality of nucleobase pairs in one or more genes using the methods provided herein results in formation of at least one intended mutation. In some embodiments, the formation of the at least one intended mutation is in the exon of a disease-associated gene and results in a restoration or increase in activity of a polypeptide encoded by the disease-associated gene. It should be appreciated that multiplex editing can be accomplished using any method or combination of methods provided herein.

The present disclosure provides methods for the treatment of a subject diagnosed with a phenylketonuria (PKU). For example, in some embodiments, a method is provided that comprises administering to a subject having or having a propensity to develop PKU, an effective amount of a nucleobase editor (e.g., an adenosine deaminase base editor or a cytidine deaminase base editor) to effect an alteration in a PKU polynucleotide sequence, thereby reducing levels of Phe in a biological sample of the subject.

### Phenylketonuria

Phenylketonuria is associated with mutations in the gene encoding PAH. In embodiments, a mutation in the PAH gene encodes a pathogenic R408W amino acid alteration to the PAH polypeptide. Phenylketonuria (commonly known as PKU) is an inherited disorder that increases the levels of a substance called phenylalanine in the blood. Phenylalanine is a protein building block (an amino acid) that is obtained from eating certain foods (such as meat, eggs, nuts, and milk) and in some artificial sweeteners. If PKU is not treated, phenylalanine can build up to harmful levels in the body, causing intellectual disability and other serious health problems.

The signs and symptoms of PKU vary from mild to severe. The most severe form of this disorder is known as classic PKU. Infants with classic PKU appear normal until they are a few months old. Without treatment, these children develop permanent intellectual disability. Seizures, delayed development, behavioral problems, and psychiatric disorders are also common. Untreated individuals may have a musty or mouse-like odor as a side effect of excess phenylalanine in the body. Children with classic PKU tend to have lighter skin and hair than unaffected family members and are also likely to have skin disorders such as eczema.

Less severe forms of this condition, sometimes called variant PKU and non-PKU hyperphenylalaninemia, have a smaller risk of brain damage. People with very mild cases may not require treatment.

PKU can often be managed by following a diet that is low in phenylalanine. Since phenylalanine is found in all proteins, the PKU diet consists of avoiding meat, dairy, nuts, tofu, and other foods that are high in protein. Infants with PKU need to be fed with a low-protein formula. Affected individuals are often limited to certain fruits and vegetables and foods containing fats and sugars (such as butter, jelly, pasta, and potato chips). The artificial sweeter aspartame, which is found in diet soda and many other low-calorie items, should be avoided as it contains high amounts of phenylalanine. The amount of phenylalanine that is safe to consume is different for each person. Affected individuals should work with a health care professional to develop an individualized diet.

Babies born to mothers who have PKU and are not following a low-phenylalanine diet have a significant risk of intellectual disability because they are exposed to very high levels of phenylalanine before birth. These infants may also have a low birth weight and grow more slowly than other children. They may also have heart defects or other heart problems, an abnormally small head size (microcephaly), and behavioral problems. Women with PKU who are not following a low-phenylalanine diet (and may have high levels of phenylalanine) also have higher risk of pregnancy loss.

Variants (also called mutations) in the PAH gene cause phenylketonuria (e.g., a mutation causing an R408W amino acid alteration in the PAH polypeptide encoded by the gene). The PAH gene provides instructions for making an enzyme called phenylalanine hydroxylase. This enzyme converts the amino acid phenylalanine into other important compounds in the body. PAH gene variants result in the production of altered versions of phenylalanine hydroxylase that cannot process phenylalanine effectively. As a result, this amino acid can build up to toxic levels in the blood and other tissues. Because nerve cells in the brain are particularly sensitive to phenylalanine levels, excessive amounts of this substance can cause brain damage.

Classic PKU, the most severe form of the disorder, occurs in people who have very low levels of phenylalanine hydroxylase activity or who have no phenylalanine hydroxylase activity at all. People with untreated classic PKU have levels of phenylalanine high enough to cause severe brain damage and other serious health problems. Variants in the PAH gene that allow the enzyme to retain some activity result in milder versions of this condition, such as variant PKU or non-PKU hyperphenylalaninemia.

Changes in other genes may influence the severity of PKU, but little is known about these additional genetic factors.

Accordingly, the present disclosure provides compositions and methods for use in the treatment of PKU in a subject. In embodiments, the methods of the disclosure are associated with a reduction in levels of phenylalanine (Phe) in a subject (e.g., in the blood of the subject). In some cases, the reduction is a 5%, 10%, 15%, 20%, 25%, 30%, 35%, 40%, 45%, 50%, 55%, 60%, 65%, 70%, 75%, 80%, 85%, 90%, or 95% reduction. In some embodiments, the reduction in Phe levels results in a Phe concentration in the blood of the subject that is below 100 μmol/L, 110 μmol/L, 120 μmol/L, 130 μmol/L, 140 μmol/L, 150 μmol/L, 160 μmol/L, 170 mol/L, 180 μmol/L, 190 μmol/L, 200 μmol/L, 250 μmol/L, 300 μmol/L, 350 μmol/L, 360 μmol/L, 400 μmol/L, 500 μmol/L, 1000 μmol/L, 1200 μmol/L, or 1500 μmol/L. In embodiments, treatment with a base editor described herein reduces the level of Phe in a biological sample of the subject to about 2-6 mg/dl (120-360 μmol/L). Methods for measuring Phe levels and/or diagnosing PKU are known in the art and described, for example, by Stone et al., “Phenylketonuria,” Treasure Island (FL): StatPearls Publishing; 2023 January; Regier et al., Phenylalanine Hydroxylase Deficiency, GeneReviews, https://www.ncbi.nlm.nih.gov/books/NBK1504/; and Vinueza, Recent Advances in Phenylketonuria: A Review, Cureus. 2023 June; 15 (6): e40459.

### Editing of Target Genes

To produce the gene edits described herein, cells (e.g., hepatocytes) are contacted with one or more guide RNAs and a nucleobase editor polypeptide (e.g., by transfecting the cells with an mRNA encoding a nucleobase editor polypeptide) comprising a nucleic acid programmable DNA binding protein (napDNAbp) and a cytidine deaminase or adenosine deaminase or comprising one or more deaminases with cytidine deaminase and/or adenosine deaminase activity (e.g., a “dual deaminase” which has cytidine and adenosine deaminase activity). In some embodiments, cells to be edited are contacted with at least one nucleic acid, wherein the at least one nucleic acid encodes one or more guide RNAs and a nucleobase editor polypeptide comprising a nucleic acid programmable DNA binding protein (napDNAbp) and a cytidine deaminase. In some embodiments, the gRNA comprises nucleotide analogs. In some instances, the gRNA is added directly to a cell. These nucleotide analogs can inhibit degradation of the gRNA from cellular processes.

Variants of the spacer sequences listed in Table 1 comprising 1, 2, 3, 4, or 5 nucleobase alterations are contemplated. For example, variation of a target polynucleotide sequence within a population (e.g., single nucleotide polymorphisms) may require said alterations to a spacer sequence to allow the spacer to better bind a variant of a target sequence in a subject.

In various instances, it is advantageous for a spacer sequence to include a 5′ and/or a 3′ “G” nucleotide. In some cases, for example, any spacer sequence or guide polynucleotide provided herein comprises or further comprises a 5′ “G”, where, in some embodiments, the 5′“G” is or is not complementary to a target sequence. In some embodiments, the 5′ “G” is added to a spacer sequence that does not already contain a 5′ “G.” For example, it can be advantageous for a guide RNA to include a 5′ terminal “G” when the guide RNA is expressed under the control of a U6 promoter or the like because the U6 promoter prefers a “G” at the transcription start site (see Cong, L. et al. “Multiplex genome engineering using CRISPR/Cas systems. Science 339:819-823 (2013) doi: 10.1126/science. 1231143). In some cases, a 5′ terminal “G” is added to a guide polynucleotide that is to be expressed under the control of a promoter but is optionally not added to the guide polynucleotide if or when the guide polynucleotide is not expressed under the control of a promoter.

Exemplary guide polynucleotide sequences are provided in the following Table 1 and exemplary base editor sequences are provided in the following Table 2.

TABLE 1
Exemplary guide polynucleotide sequences.¹

| | | SEQ | | SEQ |
|---|---|---|---|---|
| guide | | ID | | ID |
| RNA | Spacer Sequence | NO | Guide Sequence | NO |
| gRNA5847 | AAGGGCCAAGGUAUUG | 426 | mAsmAsmGsGGCCAAGGUAUUGUGGCGUU | 435 |
| | UGGC | | UUAGAGCUAGAAAUAGCAAGUUAAAAUAA | |
| | | | GGCUAGUCCGUUAUCAACUUGAAAAAGUG | |
| | | | GCACCGAGUCGGUGCmUsmUsmUsU | |
| gRNA5848 | AGGGCCAAGGUAUUGU | 427 | mAsmGsmGsGCCAAGGUAUUGUGGCAGGU | 436 |
| | GGCAG | | UUUAGUACUCUGUAAUGAAAAUUACAGAA | |
| | | | UCUACUAAAACAAGGCAAAAUGCCGUGUU | |
| | | | UAUCUCGUCAACUUGUUGGCGAGAUsmUs | |
| | | | mUsmU | |
| gRNA5849 | CCAAGGUAUUGUGGCA | 428 | mCsmCsmAsAGGUAUUGUGGCAGCAAGUU | 437 |
| | GCAA | | UUAGAGCUAGAAAUAGCAAGUUAAAAUAA | |
| | | | GGCUAGUCCGUUAUCAACUUGAAAAAGUG | |
| | | | GCACCGAGUCGGUGCmUsmUsmUsU | |
| gRNA5850 | GAAGGGCCAAGGUAUU | 429 | mGsmAsmAsGGGCCAAGGUAUUGUGGGUU | 438 |
| | GUGG | | UUAGAGCUAGAAAUAGCAAGUUAAAAUAA | |
| | | | GGCUAGUCCGUUAUCAACUUGAAAAAGUG | |
| | | | GCACCGAGUCGGUGCmUsmUsmUsU | |
| gRNA5851 | GAGAAGGGCCAAGGUA | 430 | mGsmAsmGsAAGGGCCAAGGUAUUGUGUU | 439 |
| | UUGU | | UUAGAGCUAGAAAUAGCAAGUUAAAAUAA | |
| | | | GGCUAGUCCGUUAUCAACUUGAAAAAGUG | |
| | | | GCACCGAGUCGGUGCmUsmUsmUsU | |
| gRNA5852 | GCCAAGGUAUUGUGGC | 431 | mGsmCsmCsAAGGUAUUGUGGCAGCAGUU | 440 |
| | AGCA | | UUAGAGCUAGAAAUAGCAAGUUAAAAUAA | |
| | | | GGCUAGUCCGUUAUCAACUUGAAAAAGUG | |
| | | | GCACCGAGUCGGUGCmUsmUsmUsU | |
| gRNA5853 | GGCCAAGGUAUUGUGG | 432 | mGsmGsmCsCAAGGUAUUGUGGCAGCGUU | 441 |
| | CAGC | | UUAGAGCUAGAAAUAGCAAGUUAAAAUAA | |
| | | | GGCUAGUCCGUUAUCAACUUGAAAAAGUG | |
| | | | GCACCGAGUCGGUGCmUsmUsmUsU | |
| gRNA5854 | GGGCCAAGGUAUUGUG | 433 | mGsmGsmGsCCAAGGUAUUGUGGCAGGUU | 442 |
| | GCAG | | UUAGAGCUAGAAAUAGCAAGUUAAAAUAA | |
| | | | GGCUAGUCCGUUAUCAACUUGAAAAAGUG | |
| | | | GCACCGAGUCGGUGCmUsmUsmUsU | |
| gRNA5855 | UGAGAAGGGCCAAGGU | 434 | mUsmGsmAsGAAGGGCCAAGGUAUUGGUU | 443 |
| | AUUG | | UUAGAGCUAGAAAUAGCAAGUUAAAAUAA | |
| | | | GGCUAGUCCGUUAUCAACUUGAAAAAGUG | |
| | | | GCACCGAGUCGGUGCmUsmUsmUsU | |

¹ “mN” indicates a 2′-OMe modification of the nucleotide “N”, and “Ns” indicates that the nucleotide “N” is linked to the following (i.e., 3′) nucleotide by a phosphorothioate (PS).

TABLE 2
Exemplary base editor amino acid sequences.

| | | | SEQ |
|---|---|---|---|
| MRNA | | | ID |
| Name | Editor | Base editor amino acid sequence | NO |
| MRNA4018 | ABE TadA- | MSEVEFSHEYWMRHALTLAKRARDEREVPVGAVLVLNNRVIGEGWNRA | 444 |
| | 8.20_SpCas9_ | IGLHDPTAHAEIMALRQGGLVMQNYRLYDATLYSTFEPCVMCAGAMIH | |
| | NRCH | SRIGRVVFGVRNAKTGAAGSLMDVLHHPGMNHRVEITEGILADECAAL | |
| | | LCRFFRMPRRVFNAQKKAQSSTDSGGSSGGSSGSETPGTSESATPESS | |
| | | GGSSGGSDKKYSIGLAIGTNSVGWAVITDEYKVPSKKFKVLGNTDRHS | |
| | | IKKNLIGALLFDSGETAEATRLKRTARRRYTRRKNRICYLQEIFSNEM | |
| | | AKVDDSFFHRLEESFLVEEDKKHERHPIFGNIVDEVAYHEKYPTIYHL | |
| | | RKKLVDSTDKADLRLIYLALAHMIKFRGHFLIEGDLNPDNSDVDKLFI | |
| | | QLVQTYNQLFEENPINASGVDAKAILSARLSKSRRLENLIAQLPGEKK | |
| | | NGLFGNLIALSLGLTPNFKSNFDLAEDAKLQLSKDTYDDDLDNLLAQI | |
| | | GDQYADLFLAAKNLSDAILLSDILRVNTEITKAPLSASMVKRYDEHHQ | |
| | | DLTLLKALVRQQLPEKYKEIFFDQSKNGYAGYIDGGASQEEFYKFIKP | |
| | | ILEKMDGTEELLVKLNREDLLRKQRTFDNGIIPHQIHLGELHAILRRQ | |
| | | GDFYPFLKDNREKIEKILTFRIPYYVGPLARGNSRFAWMTRKSEETIT | |
| | | PWNFEEVVDKGASAQSFIERMTNFDKNLPNEKVLPKHSLLYEYFTVYN | |
| | | ELTKVKYVTEGMRKPAFLSGEQKKAIVDLLFKTNRKVTVKQLKEDYFK | |
| | | KIECFDSVEISGVEDRFNASLGTYHDLLKIIKDKDFLDNEENEDILED | |
| | | IVLTLTLFEDREMIEERLKTYAHLFDDKVMKQLKRLRYTGWGRLSRKL | |
| | | INGIRDKQSGKTILDFLKSDGFANRNFMQLIHDDSLTFKEDIQKAQVS | |
| | | GQGDSLHEHIANLAGSPAIKKGILQTVKVVDELVKVMGGHKPENIVIE | |
| | | MARENQTTQKGQKNSRERMKRIEEGIKELGSQILKEHPVENTQLQNEK | |
| | | LYLYYLQNGRDMYVDQELDINRLSDYDVDHIVPQSFLKDDSIDNKVLT | |
| | | RSDKNRGKSDNVPSEEVVKKMKNYWRQLLNAKLITQRKFDNLTKAERG | |
| | | GLSELDKAGFIKRQLVETRQITKHVAQILDSRMNTKYDENDKLIREVK | |
| | | VITLKSKLVSDFRKDFQFYKVREINNYHHAHDAYLNAVVGTALIKKYP | |
| | | KLESEFVYGDYKVYDVRKMIAKSEQEIGKATAKYFFYSNIMNFFKTEI | |
| | | TLANGEIRKRPLIETNGETGEIVWDKGRDFATVRKVLSMPQVNIVKKT | |
| | | EVQTGGFSKESILPKGNSDKLIARKKDWDPKKYGGFNSPTVAYSVLVV | |
| | | AKVEKGKSKKLKSVKELLGITIMERSSFEKNPIDFLEAKGYKEVKKDL | |
| | | IIKLPKYSLFELENGRKRMLASAGVLQKGNELALPSKYVNFLYLASHY | |
| | | EKLKGSPEDNEQKQLFVEQHKHYLDEIIEQISEFSKRVILADANLDKV | |
| | | LSAYNKHRDKPIREQAENIIHLFTLTNLGAPAAFKYFDTTINRKQYNT | |
| | | TKEVLDATLIRQSITGLYETRIDLSQLGGDEGADKRTADGSEFESPKK | |
| | | KRKV | |
| MRNA3756 | ABE_TadA- | MKRTADGSEFESPKKKRKVSEVEFSHEYWMRHALTLAKRARDEREVPV | 445 |
| | 8.20_SpCas9_ | GAVLVLNNRVIGEGWNRAIGLHDPTAHAEIMALRQGGLVMQNYRLYDA | |
| | NG | TLYSTFEPCVMCAGAMIHSRIGRVVFGVRNAKTGAAGSLMDVLHHPGM | |
| | | NHRVEITEGILADECAALLCRFFRMPRRVFNAQKKAQSSTDSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMIKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | SIPHQIHLGELHAILRRQEDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRRRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGRHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESIRPKRNSDKLIARKKDWDP | |
| | | KKYGGFVSPTVAYSVLVVAKVEKGKSKKLKSVKELLGITIMERSSFEK | |
| | | NPIDFLEAKGYKEVKKDLIIKLPKYSLFELENGRKRMLASARFLQKGN | |
| | | ELALPSKYVNFLYLASHYEKLKGSPEDNEQKQLFVEQHKHYLDEIIEQ | |
| | | ISEFSKRVILADANLDKVLSAYNKHRDKPIREQAENIIHLFTLTNLGA | |
| | | PRAFKYFDTTIDRKVYRSTKEVLDATLIHQSITGLYETRIDLSQLGGD | |
| | | EGADKRTADGSEFESPKKKRKV | |
| MRNA3554 | ABE_8.20_ | MKRTADGSEFESPKKKRKVSEVEFSHEYWMRHALTLAKRARDEREVPV | 446 |
| | SpCas9_ | GAVLVLNNRVIGEGWNRAIGLHDPTAHAEIMALRQGGLVMQNYRLYDA | |
| | NRTH | TLYSTFEPCVMCAGAMIHSRIGRVVFGVRNAKTGAAGSLMDVLHHPGM | |
| | | NHRVEITEGILADECAALLCRFFRMPRRVFNAQKKAQSSTDSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMVKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | IIPHQIHLGELHAILRRQGDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRLRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGGHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESILPKGNSDKLIARKKDWDP | |
| | | KKYGGFNSPTVAYSVLVVAKVEKGKSKKLKSVKELLGITIMERSSFEK | |
| | | NPIGFLEAKGYKEVKKDLIIKLPKYSLFELENGRKRMLASASVLHKGN | |
| | | ELALPSKYVNFLYLASHYEKLKGSSEDNKQKQLFVEQHKHYLDEIIEQ | |
| | | ISEFSKRVILADANLDKVLSAYNKHRDKPIREQAENIIHLFTLTNLGA | |
| | | SAAFKYFDTTIGRKLYTSTKEVLDATLIHQSITGLYETRIDLSQLGGD | |
| | | EGADKRTADGSEFESPKKKRKV | |
| MRNA2743 | ABE_8.20_ | MKRTADGSEFESPKKKRKVSEVEFSHEYWMRHALTLAKRARDEREVPV | 447 |
| | SpCas9_ | GAVLVLNNRVIGEGWNRAIGLHDPTAHAEIMALRQGGLVMQNYRLYDA | |
| | NGC | TLYSTFEPCVMCAGAMIHSRIGRVVFGVRNAKTGAAGSLMDVLHHPGM | |
| | | NHRVEITEGILADECAALLCRFFRMPRRVFNAQKKAQSSTDSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMVKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | IIPHQIHLGELHAILRRQGDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRLRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGGHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESILPKGNSDKLIARKKDWDP | |
| | | KKYGGFMQPTVAYSVLVVAKVEKGKSKKLKSVKELLGITIMERSSFEK | |
| | | NPIDFLEAKGYKEVKKDLIIKLPKYSLFELENGRKRMLASAKFLQKGN | |
| | | ELALPSKYVNFLYLASHYEKLKGSPEDNEQKQLFVEQHKHYLDEIIEQ | |
| | | ISEFSKRVILADANLDKVLSAYNKHRDKPIREQAENIIHLFTLINLGA | |
| | | PRAFKYFDTTIARKEYRSTKEVLDATLIHQSITGLYETRIDLSQLGGD | |
| | | EGADKRTADGSEFESPKKKRKV | |
| MRNA3167 | ABE_8.20_ | MKRTADGSEFESPKKKRKVDKKYSIGLAIGTNSVGWAVITDEYKVPSK | 448 |
| | IBE16_ | KFKVLGNTDRHSIKKNLIGALLFDSGETAEATRLKRTARRRYTRRKNR | |
| | SpCas9_ | ICYLQEIFSNEMAKVDDSFFHRLEESFLVEEDKKHERHPIFGNIVDEV | |
| | NGC | AYHEKYPTIYHLRKKLVDSTDKADLRLIYLALAHMIKFRGHFLIEGDL | |
| | | NPDNSDVDKLFIQLVQTYNQLFEENPINASGVDAKAILSARLSKSRRL | |
| | | ENLIAQLPGEKKNGLFGNLIALSLGLTPNFKSNFDLAEDAKLQLSKDT | |
| | | YDDDLDNLLAQIGDQYADLFLAAKNLSDAILLSDILRVNTEITKAPLS | |
| | | ASMVKRYDEHHQDLTLLKALVRQQLPEKYKEIFFDQSKNGYAGYIDGG | |
| | | ASQEEFYKFIKPILEKMDGTEELLVKLNREDLLRKQRTFDNGIIPHQI | |
| | | HLGELHAILRRQGDFYPFLKDNREKIEKILTFRIPYYVGPLARGNSRF | |
| | | AWMTRKSEETITPWNFEEVVDKGASAQSFIERMTNFDKNLPNEKVLPK | |
| | | HSLLYEYFTVYNELTKVKYVTEGMRKPAFLSGEQKKAIVDLLFKTNRK | |
| | | VTVKQLKEDYFKKIECFDSVEISGVEDRFNASLGTYHDLLKIIKDKDF | |
| | | LDNEENEDILEDIVLTLTLFEDREMIEERLKTYAHLFDDKVMKQLKRL | |
| | | RYTGWGRLSRKLINGIRDKQSGKTILDFLKSDGFANRNFMQLIHDDSL | |
| | | TFKEDIQKAQVSGQGDSLHEHIANLAGSPAIKKGILQTVKVVDELVKV | |
| | | MGGHKPENIVIEMARENQTTQKGQKNSRERMKRIEEGIKELGSQILKE | |
| | | HPVENTQLQNEKLYLYYLQNGRDMYVDQELDINRLSDYDVDHIVPQSF | |
| | | LKDDSIDNKVLTRSDKNRGKSDNVPSEEVVKKMKNYWRQLLNAKLITQ | |
| | | RKFDNLTKAERGGLSELDKAGFIKRQLVETRQITKHVAQILDSRMNTK | |
| | | YDENDKLIREVKVITLKSKLVSDFRKDFQFYKVREINNYHHAHDAYLN | |
| | | AVVGTALIKKYPKLESEFVYGDYKVYDVRKMIAKSEQEIGKATAKYFF | |
| | | YSNIMNFFKTEITLANGEIRKRPLIETNGETGEIVWDKGRDFATVRKV | |
| | | LSMPQVNIVKKTEVQTGGFSKESILPKGNSDKLIARKKDWDPKKYGGF | |
| | | MQPTVAYSVLVVAKVEKGKSKKLKSVKELLGITIMERSSFEKNPIDFL | |
| | | EAKGYKEVKKDLIIKLPKYSLFELENGRKRMLASAKFLQKGNELALPS | |
| | | KYVNFLYLASHYEKLKGGSSGSETPGTSESATPESSGSEVEFSHEYWM | |
| | | RHALTLAKRARDEREVPVGAVLVLNNRVIGEGWNRAIGLHDPTAHAEI | |
| | | MALRQGGLVMQNYRLYDATLYSTFEPCVMCAGAMIHSRIGRVVFGVRN | |
| | | AKTGAAGSLMDVLHHPGMNHRVEITEGILADECAALLCRFFRMPRRVF | |
| | | NAQKKAQSSTDSPEDNEQKQLFVEQHKHYLDEIIEQISEFSKRVILAD | |
| | | ANLDKVLSAYNKHRDKPIREQAENIIHLFTLTNLGAPRAFKYFDTTIA | |
| | | RKEYRSTKEVLDATLIHQSITGLYETRIDLSQLGGDEGADKRTADGSE | |
| | | FESPKKKRKV | |
| MRNA2892 | ABE_8.20_ | MSEVEFSHEYWMRHALTLAKRARDEREVPVGAVLVLNNRVIGEGWNRA | 449 |
| | SaCas9_ | IGLHDPTAHAEIMALRQGGLVMQNYRLYDATLYSTFEPCVMCAGAMIH | |
| | NNNRRT | SRIGRVVFGVRNAKTGAAGSLMDVLHHPGMNHRVEITEGILADECAAL | |
| | | LCRFFRMPRRVFNAQKKAQSSTDSGGSSGGSSGSETPGTSESATPESS | |
| | | GGSSGGSKRNYILGLAIGITSVGYGIIDYETRDVIDAGVRLFKEANVE | |
| | | NNEGRRSKRGARRLKRRRRHRIQRVKKLLFDYNLLTDHSELSGINPYE | |
| | | ARVKGLSQKLSEEEFSAALLHLAKRRGVHNVNEVEEDTGNELSTKEQI | |
| | | SRNSKALEEKYVAELQLERLKKDGEVRGSINRFKTSDYVKEAKQLLKV | |
| | | QKAYHQLDQSFIDTYIDLLETRRTYYEGPGEGSPFGWKDIKEWYEMLM | |
| | | GHCTYFPEELRSVKYAYNADLYNALNDLNNLVITRDENEKLEYYEKFQ | |
| | | IIENVFKQKKKPTLKQIAKEILVNEEDIKGYRVTSTGKPEFTNLKVYH | |
| | | DIKDITARKEIIENAELLDQIAKILTIYQSSEDIQEELTNLNSELTQE | |
| | | EIEQISNLKGYTGTHNLSLKAINLILDELWHTNDNQIAIFNRLKLVPK | |
| | | KVDLSQQKEIPTTLVDDFILSPVVKRSFIQSIKVINAIIKKYGLPNDI | |
| | | IIELAREKNSKDAQKMINEMQKRNRQTNERIEEIIRTTGKENAKYLIE | |
| | | KIKLHDMQEGKCLYSLEAIPLEDLLNNPFNYEVDHIIPRSVSFDNSFN | |
| | | NKVLVKQEENSKKGNRTPFQYLSSSDSKISYETFKKHILNLAKGKGRI | |
| | | SKTKKEYLLEERDINRFSVQKDFINRNLVDTRYATRGLMNLLRSYFRV | |
| | | NNLDVKVKSINGGFTSFLRRKWKFKKERNKGYKHHAEDALIIANADFI | |
| | | FKEWKKLDKAKKVMENQMFEEKQAESMPEIETEQEYKEIFITPHQIKH | |
| | | IKDFKDYKYSHRVDKKPNRKLINDTLYSTRKDDKGNTLIVNNLNGLYD | |
| | | KDNDKLKKLINKSPEKLLMYHHDPQTYQKLKLIMEQYGDEKNPLYKYY | |
| | | EETGNYLTKYSKKDNGPVIKKIKYYGNKLNAHLDITDDYPNSRNKVVK | |
| | | LSLKPYRFDVYLDNGVYKFVTVKNLDVIKKENYYEVNSKCYEEAKKLK | |
| | | KISNQAEFIASFYKNDLIKINGELYRVIGVNNDLLNRIEVNMIDITYR | |
| | | EYLENMNDKRPPHIIKTIASKTQSIKKYSTDILGNLYEVKSKKHPQII | |
| | | KKGGSPKKKRKVSSDYKDHDGDYKDHDIDYKDDDDKEGADKRTADGSE | |
| | | FESPKKKRKV | |
| MRNA4681 | ABE_9.51_ | MSEVEFSHEYWMRHALTLAKRARDEREVPVGAVLVLNNRVIGEGWNRA | 450 |
| | spCas9_NRN | IGLHDPTAHAEIMALRQGGLVMQNYRLYDATLYTTFEPCVMCAGAMIH | |
| | | SRIGRVVFGVRNAKTGAAGSLMDVLHHPGMNHRVEITEGILADECAAL | |
| | | LCRFFRMPRRVFNAQKKAQSSTDSGGSSGGSSGSETPGTSESATPESS | |
| | | GGSSGGSDKKYSIGLAIGTNSVGWAVITDEYKVPSKKFKVLGNTDRHS | |
| | | IKKNLIGALLFDSGETAERTRLKRTARRRYTRRKNRICYLQEIFSNEM | |
| | | AKVDDSFFHRLEESFLVEEDKKHERHPIFGNIVDEVAYHEKYPTIYHL | |
| | | RKKLVDSTDKADLRLIYLALAHMIKFRGHFLIEGDLNPDNSDVDKLFI | |
| | | QLVQTYNQLFEENPINASGVDAKAILSARLSKSRRLENLIAQLPGEKK | |
| | | NGLFGNLIALSLGLTPNFKSNFDLAEDAKLQLSKDTYDDDLDNLLAQI | |
| | | GDQYADLFLAAKNLSDAILLSDILRVNTEITKAPLSASMIKRYDEHHQ | |
| | | DLTLLKALVRQQLPEKYKEIFFDQSKNGYAGYIDGGASQEEFYKFIKP | |
| | | ILEKMDGTEELLVKLNREDLLRKQRTFDNGSIPHQIHLGELHAILRRQ | |
| | | EDFYPFLKDNREKIEKILTFRIPYYVGPLARGNSRFAWMTRKSEETIT | |
| | | PWNFEEVVDKGASAQSFIERMTNFDKNLPNEKVLPKHSLLYEYFTVYN | |
| | | ELTKVKYVTEGMRKPAFLSGEQKKAIVDLLFKTNRKVTVKQLKEDYFK | |
| | | KIECFDSVEISGVEDRFNASLGTYHDLLKIIKDKDFLDNEENEDILED | |
| | | IVLTLTLFEDREMIEERLKTYAHLFDDKVMKQLKRRRYTGWGRLSRKL | |
| | | INGIRDKQSGKTILDFLKSDGFANRNFMQLIHDDSLTFKEDIQKAQVS | |
| | | GQGDSLHEHIANLAGSPAIKKGILQTVKVVDELVKVMGRHKPENIVIE | |
| | | MARENQTTQKGQKNSRERMKRIEEGIKELGSQILKEHPVENTQLQNEK | |
| | | LYLYYLQNGRDMYVDQELDINRLSDYDVDHIVPQSFLKDDSIDNKVLT | |
| | | RSDKNRGKSDNVPSEEVVKKMKNYWRQLLNAKLITQRKFDNLTKAERG | |
| | | GLSELDKAGFIKRQLVETRQITKHVAQILDSRMNTKYDENDKLIREVK | |
| | | VITLKSKLVSDFRKDFQFYKVREINNYHHAHDAYLNAVVGTALIKKYP | |
| | | KLESEFVYGDYKVYDVRKMIAKSEQEIGKATAKYFFYSNIMNFFKTEI | |
| | | TLANGEIRKRPLIETNGETGEIVWDKGRDFATVRKVLSMPQVNIVKKT | |
| | | EVQTGGFSKESIRPKRNSDKLIARKKDWDPKKYGGFLWPTVAYSVLVV | |
| | | AKVEKGKSKKLKSVKELLGITIMERSSFEKNPIDFLEAKGYKEVKKDL | |
| | | IIKLPKYSLFELENGRKRMLASAKQLQKGNELALPSKYVNFLYLASHY | |
| | | EKLKGSPEDNEQKQLFVEQHKHYLDEIIEQISEFSKRVILADANLDKV | |
| | | LSAYNKHRDKPIREQAENIIHLFTLTRLGAPRAFKYFDTTIDPKQYRS | |
| | | TKEVLDATLIHQSITGLYETRIDLSQLGGDEGADKRTADGSEFESPKK | |
| | | KRKV | |
| MRNA3169 | ABE_8.20_IBE | MKRTADGSEFESPKKKRKVDKKYSIGLAIGTNSVGWAVITDEYKVPSK | 451 |
| | 16_SpCas9_N | KFKVLGNTDRHSIKKNLIGALLFDSGETAEATRLKRTARRRYTRRKNR | |
| | GG | ICYLQEIFSNEMAKVDDSFFHRLEESFLVEEDKKHERHPIFGNIVDEV | |
| | | AYHEKYPTIYHLRKKLVDSTDKADLRLIYLALAHMIKFRGHFLIEGDL | |
| | | NPDNSDVDKLFIQLVQTYNQLFEENPINASGVDAKAILSARLSKSRRL | |
| | | ENLIAQLPGEKKNGLFGNLIALSLGLTPNFKSNFDLAEDAKLQLSKDT | |
| | | YDDDLDNLLAQIGDQYADLFLAAKNLSDAILLSDILRVNTEITKAPLS | |
| | | ASMIKRYDEHHQDLTLLKALVRQQLPEKYKEIFFDQSKNGYAGYIDGG | |
| | | ASQEEFYKFIKPILEKMDGTEELLVKLNREDLLRKQRTFDNGSIPHQI | |
| | | HLGELHAILRRQEDFYPFLKDNREKIEKILTFRIPYYVGPLARGNSRF | |
| | | AWMTRKSEETITPWNFEEVVDKGASAQSFIERMTNFDKNLPNEKVLPK | |
| | | HSLLYEYFTVYNELTKVKYVTEGMRKPAFLSGEQKKAIVDLLFKTNRK | |
| | | VTVKQLKEDYFKKIECFDSVEISGVEDRFNASLGTYHDLLKIIKDKDF | |
| | | LDNEENEDILEDIVLTLTLFEDREMIEERLKTYAHLFDDKVMKQLKRR | |
| | | RYTGWGRLSRKLINGIRDKQSGKTILDFLKSDGFANRNFMQLIHDDSL | |
| | | TFKEDIQKAQVSGQGDSLHEHIANLAGSPAIKKGILQTVKVVDELVKV | |
| | | MGRHKPENIVIEMARENQTTQKGQKNSRERMKRIEEGIKELGSQILKE | |
| | | HPVENTQLQNEKLYLYYLQNGRDMYVDQELDINRLSDYDVDHIVPQSF | |
| | | LKDDSIDNKVLTRSDKNRGKSDNVPSEEVVKKMKNYWRQLLNAKLITQ | |
| | | RKFDNLTKAERGGLSELDKAGFIKRQLVETRQITKHVAQILDSRMNTK | |
| | | YDENDKLIREVKVITLKSKLVSDFRKDFQFYKVREINNYHHAHDAYLN | |
| | | AVVGTALIKKYPKLESEFVYGDYKVYDVRKMIAKSEQEIGKATAKYFF | |
| | | YSNIMNFFKTEITLANGEIRKRPLIETNGETGEIVWDKGRDFATVRKV | |
| | | LSMPQVNIVKKTEVQTGGFSKESILPKRNSDKLIARKKDWDPKKYGGF | |
| | | DSPTVAYSVLVVAKVEKGKSKKLKSVKELLGITIMERSSFEKNPIDFL | |
| | | EAKGYKEVKKDLIIKLPKYSLFELENGRKRMLASAGELQKGNELALPS | |
| | | KYVNFLYLASHYEKLKGGSSGSETPGTSESATPESSGSEVEFSHEYWM | |
| | | RHALTLAKRARDEREVPVGAVLVLNNRVIGEGWNRAIGLHDPTAHAEI | |
| | | MALRQGGLVMQNYRLYDATLYSTFEPCVMCAGAMIHSRIGRVVFGVRN | |
| | | AKTGAAGSLMDVLHHPGMNHRVEITEGILADECAALLCRFFRMPRRVF | |
| | | NAQKKAQSSTDSPEDNEQKQLFVEQHKHYLDEIIEQISEFSKRVILAD | |
| | | ANLDKVLSAYNKHRDKPIREQAENIIHLFTLTNLGAPAAFKYFDTTID | |
| | | RKRYTSTKEVLDATLIHQSITGLYETRIDLSQLGGDEGADKRTADGSE | |
| | | FESPKKKRKV | |
| MRNA3170 | ABE_8.20_IBE | MKRTADGSEFESPKKKRKVDKKYSIGLAIGTNSVGWAVITDEYKVPSK | 452 |
| | 12_SpCas9_N | KFKVLGNTDRHSIKKNLIGALLFDSGETAEATRLKRTARRRYTRRKNR | |
| | GC | ICYLQEIFSNEMAKVDDSFFHRLEESFLVEEDKKHERHPIFGNIVDEV | |
| | | AYHEKYPTIYHLRKKLVDSTDKADLRLIYLALAHMIKFRGHFLIEGDL | |
| | | NPDNSDVDKLFIQLVQTYNQLFEENPINASGVDAKAILSARLSKSRRL | |
| | | ENLIAQLPGEKKNGLFGNLIALSLGLTPNFKSNFDLAEDAKLQLSKDT | |
| | | YDDDLDNLLAQIGDQYADLFLAAKNLSDAILLSDILRVNTEITKAPLS | |
| | | ASMVKRYDEHHQDLTLLKALVRQQLPEKYKEIFFDQSKNGYAGYIDGG | |
| | | ASQEEFYKFIKPILEKMDGTEELLVKLNREDLLRKQRTFDNGIIPHQI | |
| | | HLGELHAILRRQGDFYPFLKDNREKIEKILTFRIPYYVGPLARGNSRF | |
| | | AWMTRKSEETITPWNFEEVVDKGASAQSFIERMTNFDKNLPNEKVLPK | |
| | | HSLLYEYFTVYNELTKVKYVTEGMRKPAFLSGEQKKAIVDLLFKTNRK | |
| | | VTVKQLKEDYFKKIECFDSVEISGVEDRFNASLGTYHDLLKIIKDKDF | |
| | | LDNEENEDILEDIVLTLTLFEDREMIEERLKTYAHLFDDKVMKQLKRL | |
| | | RYTGWGRLSRKLINGIRDKQSGKTILDFLKSDGFANRNFMQLIHDDSL | |
| | | TFKEDIQKAQVSGQGDSLHEHIANLAGSPAIKKGILQTVKVVDELVKV | |
| | | MGGHKPENIVIEMARENQTTQKGQKNSRERMKRIEEGIKELGSQILKE | |
| | | HPVENTQLQNEKLYLYYLQNGRDMYVDQELDINRLSDYDVDHIVPQSF | |
| | | LKDDSIDNKVLTRSDKNRGKSDNVPSEEVVKKMKNYWRQLLNAKLITQ | |
| | | RKFDNLTKAERGGLSELDKAGFIKRQLVETRQITKHVAQILDSRMNTK | |
| | | YDENDKLIREVKVITLKSKLVSDFRKDFQFYKVREINNYHHAHDAYLN | |
| | | AVVGTALIKKYPKLESEFVYGDYKVYDVRKMIAKSEQEIGSSGSETPG | |
| | | TSESATPESSGSEVEFSHEYWMRHALTLAKRARDEREVPVGAVLVLNN | |
| | | RVIGEGWNRAIGLHDPTAHAEIMALRQGGLVMQNYRLYDATLYSTFEP | |
| | | CVMCAGAMIHSRIGRVVFGVRNAKTGAAGSLMDVLHHPGMNHRVEITE | |
| | | GILADECAALLCRFFRMPRRVFNAQKKAQSSTDGKATAKYFFYSNIMN | |
| | | FFKTEITLANGEIRKRPLIETNGETGEIVWDKGRDFATVRKVLSMPQV | |
| | | NIVKKTEVQTGGFSKESILPKGNSDKLIARKKDWDPKKYGGFMQPTVA | |
| | | YSVLVVAKVEKGKSKKLKSVKELLGITIMERSSFEKNPIDFLEAKGYK | |
| | | EVKKDLIIKLPKYSLFELENGRKRMLASAKFLQKGNELALPSKYVNFL | |
| | | YLASHYEKLKGSPEDNEQKQLFVEQHKHYLDEIIEQISEFSKRVILAD | |
| | | ANLDKVLSAYNKHRDKPIREQAENIIHLFTLTNLGAPRAFKYFDTTIA | |
| | | RKEYRSTKEVLDATLIHQSITGLYETRIDLSQLGGDEGADKRTADGSE | |
| | | FESPKKKRKV | |
| MRNA4022 | ABE_TadA- | MSEVEFSHEYWMRHALTLAKRARDEREVPVGAVLVLNNRVIGEGWNRA | 453 |
| | 9.51_SpCas9 | IGLHDPTAHAEIMALRQGGLVMQNYRLYDATLYTTFEPCVMCAGAMIH | |
| | NRCH | SRIGRVVFGVRNAKTGAAGSLMDVLHHPGMNHRVEITEGILADECAAL | |
| | | LCRFFRMPRRVFNAQKKAQSSTDSGGSSGGSSGSETPGTSESATPESS | |
| | | GGSSGGSDKKYSIGLAIGTNSVGWAVITDEYKVPSKKFKVLGNTDRHS | |
| | | IKKNLIGALLFDSGETAEATRLKRTARRRYTRRKNRICYLQEIFSNEM | |
| | | AKVDDSFFHRLEESFLVEEDKKHERHPIFGNIVDEVAYHEKYPTIYHL | |
| | | RKKLVDSTDKADLRLIYLALAHMIKFRGHFLIEGDLNPDNSDVDKLFI | |
| | | QLVQTYNQLFEENPINASGVDAKAILSARLSKSRRLENLIAQLPGEKK | |
| | | NGLFGNLIALSLGLTPNFKSNFDLAEDAKLQLSKDTYDDDLDNLLAQI | |
| | | GDQYADLFLAAKNLSDAILLSDILRVNTEITKAPLSASMVKRYDEHHQ | |
| | | DLTLLKALVRQQLPEKYKEIFFDQSKNGYAGYIDGGASQEEFYKFIKP | |
| | | ILEKMDGTEELLVKLNREDLLRKQRTFDNGIIPHQIHLGELHAILRRQ | |
| | | GDFYPFLKDNREKIEKILTFRIPYYVGPLARGNSRFAWMTRKSEETIT | |
| | | PWNFEEVVDKGASAQSFIERMTNFDKNLPNEKVLPKHSLLYEYFTVYN | |
| | | ELTKVKYVTEGMRKPAFLSGEQKKAIVDLLFKTNRKVTVKQLKEDYFK | |
| | | KIECFDSVEISGVEDRFNASLGTYHDLLKIIKDKDFLDNEENEDILED | |
| | | IVLTLTLFEDREMIEERLKTYAHLFDDKVMKQLKRLRYTGWGRLSRKL | |
| | | INGIRDKQSGKTILDFLKSDGFANRNFMQLIHDDSLTFKEDIQKAQVS | |
| | | GQGDSLHEHIANLAGSPAIKKGILQTVKVVDELVKVMGGHKPENIVIE | |
| | | MARENQTTQKGQKNSRERMKRIEEGIKELGSQILKEHPVENTQLQNEK | |
| | | LYLYYLQNGRDMYVDQELDINRLSDYDVDHIVPQSFLKDDSIDNKVLT | |
| | | RSDKNRGKSDNVPSEEVVKKMKNYWRQLLNAKLITQRKFDNLTKAERG | |
| | | GLSELDKAGFIKRQLVETRQITKHVAQILDSRMNTKYDENDKLIREVK | |
| | | VITLKSKLVSDFRKDFQFYKVREINNYHHAHDAYLNAVVGTALIKKYP | |
| | | KLESEFVYGDYKVYDVRKMIAKSEQEIGKATAKYFFYSNIMNFFKTEI | |
| | | TLANGEIRKRPLIETNGETGEIVWDKGRDFATVRKVLSMPQVNIVKKT | |
| | | EVQTGGFSKESILPKGNSDKLIARKKDWDPKKYGGFNSPTVAYSVLVV | |
| | | AKVEKGKSKKLKSVKELLGITIMERSSFEKNPIDFLEAKGYKEVKKDL | |
| | | IIKLPKYSLFELENGRKRMLASAGVLQKGNELALPSKYVNFLYLASHY | |
| | | EKLKGSPEDNEQKQLFVEQHKHYLDEIIEQISEFSKRVILADANLDKV | |
| | | LSAYNKHRDKPIREQAENIIHLFTLTNLGAPAAFKYFDTTINRKQYNT | |
| | | TKEVLDATLIRQSITGLYETRIDLSQLGGDEGADKRTADGSEFESPKK | |
| | | KRKV | |
| MRNA4232 | ABE_9.1_SpC | MKRTADGSEFESPKKKRKVSEVEFSHEYWMRHALTLAKRARDEREVPV | 454 |
| | as9_NGC | GAVLVLNNRVIGEGWNRAIGLHDPTAHAEIMALRQGGLVMQNYRLYDA | |
| | | TLYTTFEPCVMCAGAMIHSRIGRVVFGVRNAKTGAAGSLMDVLHHPGM | |
| | | NHRVEITEGILADECAALLCRFYRMPRRVFNAQKKAQSSTDSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMVKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | IIPHQIHLGELHAILRRQGDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRLRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGGHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESILPKGNSDKLIARKKDWDP | |
| | | KKYGGFMQPTVAYSVLVVAKVEKGKSKKLKSVKELLGITIMERSSFEK | |
| | | NPIDFLEAKGYKEVKKDLIIKLPKYSLFELENGRKRMLASAKFLQKGN | |
| | | ELALPSKYVNFLYLASHYEKLKGSPEDNEQKQLFVEQHKHYLDEIIEQ | |
| | | ISEFSKRVILADANLDKVLSAYNKHRDKPIREQAENIIHLFTLTNLGA | |
| | | PRAFKYFDTTIARKEYRSTKEVLDATLIHQSITGLYETRIDLSQLGGD | |
| | | EGADKRTADGSEFESPKKKRKV | |
| MRNA4233 | ABE_9.2_SpC | MKRTADGSEFESPKKKRKVSEVEFSHEYWMRHALTLAKRARDEREVPV | 455 |
| | as9_NGC | GAVLVLNNRVIGEGWNRAIGLHDPTAHAEIMALRQGGLVMQNYRLYDA | |
| | | TLYTTFEPCVMCAGAMIHSRIGRVVFGVRNAKTGAAGSLMDVLHHPGM | |
| | | NHRVEITEGILADECAALLCDFYRMPRRVFNAQKKAQSSINSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMVKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | IIPHQIHLGELHAILRRQGDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRLRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGGHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESILPKGNSDKLIARKKDWDP | |
| | | KKYGGFMQPTVAYSVLVVAKVEKGKSKKLKSVKELLGITIMERSSFEK | |
| | | NPIDFLEAKGYKEVKKDLIIKLPKYSLFELENGRKRMLASAKFLQKGN | |
| | | ELALPSKYVNFLYLASHYEKLKGSPEDNEQKQLFVEQHKHYLDEIIEQ | |
| | | ISEFSKRVILADANLDKVLSAYNKHRDKPIREQAENIIHLFTLTNLGA | |
| | | PRAFKYFDTTIARKEYRSTKEVLDATLIHQSITGLYETRIDLSQLGGD | |
| | | EGADKRTADGSEFESPKKKRKV | |
| MRNA4234 | ABE_9.1_SpC | MKRTADGSEFESPKKKRKVSEVEFSHEYWMRHALTLAKRARDEREVPV | 456 |
| | as9_NG | GAVLVLNNRVIGEGWNRAIGLHDPTAHAEIMALRQGGLVMQNYRLYDA | |
| | | TLYTTFEPCVMCAGAMIHSRIGRVVFGVRNAKTGAAGSLMDVLHHPGM | |
| | | NHRVEITEGILADECAALLCRFYRMPRRVFNAQKKAQSSTDSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMIKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | SIPHQIHLGELHAILRRQEDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRRRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGRHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESIRPKRNSDKLIARKKDWDP | |
| | | KKYGGFVSPTVAYSVLVVAKVEKGKSKKLKSVKELLGITIMERSSFEK | |
| | | NPIDFLEAKGYKEVKKDLIIKLPKYSLFELENGRKRMLASARFLQKGN | |
| | | ELALPSKYVNFLYLASHYEKLKGSPEDNEQKQLFVEQHKHYLDEIIEQ | |
| | | ISEFSKRVILADANLDKVLSAYNKHRDKPIREQAENIIHLFTLTNLGA | |
| | | PRAFKYFDTTIDRKAYRSTKEVLDATLIHQSITGLYETRIDLSQLGGD | |
| | | EGADKRTADGSEFESPKKKRKV | |
| MRNA4235 | ABE_9.2_SpC | MKRTADGSEFESPKKKRKVSEVEFSHEYWMRHALTLAKRARDEREVPV | 457 |
| | as9_NG | GAVLVLNNRVIGEGWNRAIGLHDPTAHAEIMALRQGGLVMQNYRLYDA | |
| | | TLYTTFEPCVMCAGAMIHSRIGRVVFGVRNAKTGAAGSLMDVLHHPGM | |
| | | NHRVEITEGILADECAALLCDFYRMPRRVFNAQKKAQSSINSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMIKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | SIPHQIHLGELHAILRRQEDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRRRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGRHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESIRPKRNSDKLIARKKDWDP | |
| | | KKYGGFVSPTVAYSVLVVAKVEKGKSKKLKSVKELLGITIMERSSFEK | |
| | | NPIDFLEAKGYKEVKKDLIIKLPKYSLFELENGRKRMLASARFLQKGN | |
| | | ELALPSKYVNFLYLASHYEKLKGSPEDNEQKQLFVEQHKHYLDEIIEQ | |
| | | ISEFSKRVILADANLDKVLSAYNKHRDKPIREQAENIIHLFTLTNLGA | |
| | | PRAFKYFDTTIDRKAYRSTKEVLDATLIHQSITGLYETRIDLSQLGGD | |
| | | EGADKRTADGSEFESPKKKRKV | |
| MRNA4684 | ABE_TadA- | MSEVEFSHEYWMRHALTLAKRARDEREVPVGAVLVLNNRVIGEGWNRA | 458 |
| | 9.51_SpCas9 | IGLHDPTAHAEIMALRQGGLVMQNYRLYDATLYTTFEPCVMCAGAMIH | |
| | NG | SRIGRVVFGVRNAKTGAAGSLMDVLHHPGMNHRVEITEGILADECAAL | |
| | | LCRFFRMPRRVFNAQKKAQSSTDSGGSSGGSSGSETPGTSESATPESS | |
| | | GGSSGGSDKKYSIGLAIGTNSVGWAVITDEYKVPSKKFKVLGNTDRHS | |
| | | IKKNLIGALLFDSGETAEATRLKRTARRRYTRRKNRICYLQEIFSNEM | |
| | | AKVDDSFFHRLEESFLVEEDKKHERHPIFGNIVDEVAYHEKYPTIYHL | |
| | | RKKLVDSTDKADLRLIYLALAHMIKFRGHFLIEGDLNPDNSDVDKLFI | |
| | | QLVQTYNQLFEENPINASGVDAKAILSARLSKSRRLENLIAQLPGEKK | |
| | | NGLFGNLIALSLGLTPNFKSNFDLAEDAKLQLSKDTYDDDLDNLLAQI | |
| | | GDQYADLFLAAKNLSDAILLSDILRVNTEITKAPLSASMIKRYDEHHQ | |
| | | DLTLLKALVRQQLPEKYKEIFFDQSKNGYAGYIDGGASQEEFYKFIKP | |
| | | ILEKMDGTEELLVKLNREDLLRKQRTFDNGSIPHQIHLGELHAILRRQ | |
| | | EDFYPFLKDNREKIEKILTFRIPYYVGPLARGNSRFAWMTRKSEETIT | |
| | | PWNFEEVVDKGASAQSFIERMTNFDKNLPNEKVLPKHSLLYEYFTVYN | |
| | | ELTKVKYVTEGMRKPAFLSGEQKKAIVDLLFKTNRKVTVKQLKEDYFK | |
| | | KIECFDSVEISGVEDRFNASLGTYHDLLKIIKDKDFLDNEENEDILED | |
| | | IVLTLTLFEDREMIEERLKTYAHLFDDKVMKQLKRRRYTGWGRLSRKL | |
| | | INGIRDKQSGKTILDFLKSDGFANRNFMQLIHDDSLTFKEDIQKAQVS | |
| | | GQGDSLHEHIANLAGSPAIKKGILQTVKVVDELVKVMGRHKPENIVIE | |
| | | MARENQTTQKGQKNSRERMKRIEEGIKELGSQILKEHPVENTQLQNEK | |
| | | LYLYYLQNGRDMYVDQELDINRLSDYDVDHIVPQSFLKDDSIDNKVLT | |
| | | RSDKNRGKSDNVPSEEVVKKMKNYWRQLLNAKLITQRKFDNLTKAERG | |
| | | GLSELDKAGFIKRQLVETRQITKHVAQILDSRMNTKYDENDKLIREVK | |
| | | VITLKSKLVSDFRKDFQFYKVREINNYHHAHDAYLNAVVGTALIKKYP | |
| | | KLESEFVYGDYKVYDVRKMIAKSEQEIGKATAKYFFYSNIMNFFKTEI | |
| | | TLANGEIRKRPLIETNGETGEIVWDKGRDFATVRKVLSMPQVNIVKKT | |
| | | EVQTGGFSKESILPKRNSDKLIARKKDWDPKKYGGFLWPTVAYSVLVV | |
| | | AKVEKGKSKKLKSVKELLGITIMERSSFEKNPIDFLEAKGYKEVKKDL | |
| | | IIKLPKYSLFELENGRKRMLASAKQLQKGNELALPSKYVNFLYLASHY | |
| | | EKLKGSPEDNEQKQLFVEQHKHYLDEIIEQISEFSKRVILADANLDKV | |
| | | LSAYNKHRDKPIREQAENIIHLFTLTNLGAPAAFKYFDTTIDRKQYRS | |
| | | TKEVLDATLIHQSITGLYETRIDLSQLGGDEGADKRTADGSEFESPKK | |
| | | KRKV | |
| MRNA3921 | ABE_9.51_Sp | MKRTADGSEFESPKKKRKVSEVEFSHEYWMRHALTLAKRARDEREVPV | 459 |
| | Cas9_NRTH | GAVLVLNNRVIGEGWNRAIGLHDPTAHAEIMALRQGGLVMQNYRLYDA | |
| | | TLYTTFEPCVMCAGAMIHSRIGRVVFGVRNAKTGAAGSLMDVLHHPGM | |
| | | NHRVEITEGILADECAALLCRFFRMPRRVFNAQKKAQSSTDSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMVKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | IIPHQIHLGELHAILRRQGDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRLRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGGHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESILPKGNSDKLIARKKDWDP | |
| | | KKYGGFNSPTVAYSVLVVAKVEKGKSKKLKSVKELLGITIMERSSFEK | |
| | | NPIGFLEAKGYKEVKKDLIIKLPKYSLFELENGRKRMLASASVLHKGN | |
| | | ELALPSKYVNFLYLASHYEKLKGSSEDNKQKQLFVEQHKHYLDEIIEQ | |
| | | ISEFSKRVILADANLDKVLSAYNKHRDKPIREQAENIIHLFTLTNLGA | |
| | | SAAFKYFDTTIGRKLYTSTKEVLDATLIHQSITGLYETRIDLSQLGGD | |
| | | EGADKRTADGSEFESPKKKRKV | |
| MRNA4236 | ABE_9.1_SpC | MKRTADGSEFESPKKKRKVSEVEFSHEYWMRHALTLAKRARDEREVPV | 460 |
| | as9_NRTH | GAVLVLNNRVIGEGWNRAIGLHDPTAHAEIMALRQGGLVMQNYRLYDA | |
| | | TLYTTFEPCVMCAGAMIHSRIGRVVFGVRNAKTGAAGSLMDVLHHPGM | |
| | | NHRVEITEGILADECAALLCRFYRMPRRVFNAQKKAQSSTDSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMVKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | IIPHQIHLGELHAILRRQGDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRLRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGGHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESILPKGNSDKLIARKKDWDP | |
| | | KKYGGFNSPTVAYSVLVVAKVEKGKSKKLKSVKELLGITIMERSSFEK | |
| | | NPIGFLEAKGYKEVKKDLIIKLPKYSLFELENGRKRMLASASVLHKGN | |
| | | ELALPSKYVNFLYLASHYEKLKGSSEDNKQKQLFVEQHKHYLDEIIEQ | |
| | | ISEFSKRVILADANLDKVLSAYNKHRDKPIREQAENIIHLFTLTNLGA | |
| | | SAAFKYFDTTIGRKLYTSTKEVLDATLIHQSITGLYETRIDLSQLGGD | |
| | | EGADKRTADGSEFESPKKKRKV | |
| MRNA4237 | ABE_9.2_SpC | MKRTADGSEFESPKKKRKVSEVEFSHEYWMRHALTLAKRARDEREVPV | 461 |
| | as9_NRTH | GAVLVLNNRVIGEGWNRAIGLHDPTAHAEIMALRQGGLVMQNYRLYDA | |
| | | TLYTTFEPCVMCAGAMIHSRIGRVVFGVRNAKTGAAGSLMDVLHHPGM | |
| | | NHRVEITEGILADECAALLCDFYRMPRRVFNAQKKAQSSINSGGSSGG | |
| | | SSGSETPGTSESATPESSGGSSGGSDKKYSIGLAIGTNSVGWAVITDE | |
| | | YKVPSKKFKVLGNTDRHSIKKNLIGALLFDSGETAEATRLKRTARRRY | |
| | | TRRKNRICYLQEIFSNEMAKVDDSFFHRLEESFLVEEDKKHERHPIFG | |
| | | NIVDEVAYHEKYPTIYHLRKKLVDSTDKADLRLIYLALAHMIKFRGHF | |
| | | LIEGDLNPDNSDVDKLFIQLVQTYNQLFEENPINASGVDAKAILSARL | |
| | | SKSRRLENLIAQLPGEKKNGLFGNLIALSLGLTPNFKSNFDLAEDAKL | |
| | | QLSKDTYDDDLDNLLAQIGDQYADLFLAAKNLSDAILLSDILRVNTEI | |
| | | TKAPLSASMVKRYDEHHQDLTLLKALVRQQLPEKYKEIFFDQSKNGYA | |
| | | GYIDGGASQEEFYKFIKPILEKMDGTEELLVKLNREDLLRKQRTFDNG | |
| | | IIPHQIHLGELHAILRRQGDFYPFLKDNREKIEKILTFRIPYYVGPLA | |
| | | RGNSRFAWMTRKSEETITPWNFEEVVDKGASAQSFIERMTNFDKNLPN | |
| | | EKVLPKHSLLYEYFTVYNELTKVKYVTEGMRKPAFLSGEQKKAIVDLL | |
| | | FKTNRKVTVKQLKEDYFKKIECFDSVEISGVEDRFNASLGTYHDLLKI | |
| | | IKDKDFLDNEENEDILEDIVLTLTLFEDREMIEERLKTYAHLFDDKVM | |
| | | KQLKRLRYTGWGRLSRKLINGIRDKQSGKTILDFLKSDGFANRNFMQL | |
| | | IHDDSLTFKEDIQKAQVSGQGDSLHEHIANLAGSPAIKKGILQTVKVV | |
| | | DELVKVMGGHKPENIVIEMARENQTTQKGQKNSRERMKRIEEGIKELG | |
| | | SQILKEHPVENTQLQNEKLYLYYLQNGRDMYVDQELDINRLSDYDVDH | |
| | | IVPQSFLKDDSIDNKVLTRSDKNRGKSDNVPSEEVVKKMKNYWRQLLN | |
| | | AKLITQRKFDNLTKAERGGLSELDKAGFIKRQLVETRQITKHVAQILD | |
| | | SRMNTKYDENDKLIREVKVITLKSKLVSDFRKDFQFYKVREINNYHHA | |
| | | HDAYLNAVVGTALIKKYPKLESEFVYGDYKVYDVRKMIAKSEQEIGKA | |
| | | TAKYFFYSNIMNFFKTEITLANGEIRKRPLIETNGETGEIVWDKGRDF | |
| | | ATVRKVLSMPQVNIVKKTEVQTGGFSKESILPKGNSDKLIARKKDWDP | |
| | | KKYGGFNSPTVAYSVLVVAKVEKGKSKKLKSVKELLGITIMERSSFEK | |
| | | NPIGFLEAKGYKEVKKDLIIKLPKYSLFELENGRKRMLASASVLHKGN | |
| | | ELALPSKYVNFLYLASHYEKLKGSSEDNKQKQLFVEQHKHYLDEIIEQ | |
| | | ISEFSKRVILADANLDKVLSAYNKHRDKPIREQAENIIHLFTLTNLGA | |
| | | SAAFKYFDTTIGRKLYTSTKEVLDATLIHQSITGLYETRIDLSQLGGD | |
| | | EGADKRTADGSEFESPKKKRKV | |
| MRNA2960 | ABE_8.20_IBE | MKRTADGSEFESPKKKRKVDKKYSIGLAIGTNSVGWAVITDEYKVPSK | 462 |
| | 12_SpCas9_N | KFKVLGNTDRHSIKKNLIGALLFDSGETAEATRLKRTARRRYTRRKNR | |
| | GG | ICYLQEIFSNEMAKVDDSFFHRLEESFLVEEDKKHERHPIFGNIVDEV | |
| | | AYHEKYPTIYHLRKKLVDSTDKADLRLIYLALAHMIKFRGHFLIEGDL | |
| | | NPDNSDVDKLFIQLVQTYNQLFEENPINASGVDAKAILSARLSKSRRL | |
| | | ENLIAQLPGEKKNGLFGNLIALSLGLTPNFKSNFDLAEDAKLQLSKDT | |
| | | YDDDLDNLLAQIGDQYADLFLAAKNLSDAILLSDILRVNTEITKAPLS | |
| | | ASMIKRYDEHHQDLTLLKALVRQQLPEKYKEIFFDQSKNGYAGYIDGG | |
| | | ASQEEFYKFIKPILEKMDGTEELLVKLNREDLLRKQRTFDNGSIPHQI | |
| | | HLGELHAILRRQEDFYPFLKDNREKIEKILTFRIPYYVGPLARGNSRF | |
| | | AWMTRKSEETITPWNFEEVVDKGASAQSFIERMTNFDKNLPNEKVLPK | |
| | | HSLLYEYFTVYNELTKVKYVTEGMRKPAFLSGEQKKAIVDLLFKTNRK | |
| | | VTVKQLKEDYFKKIECFDSVEISGVEDRFNASLGTYHDLLKIIKDKDF | |
| | | LDNEENEDILEDIVLTLTLFEDREMIEERLKTYAHLFDDKVMKQLKRR | |
| | | RYTGWGRLSRKLINGIRDKQSGKTILDFLKSDGFANRNFMQLIHDDSL | |
| | | TFKEDIQKAQVSGQGDSLHEHIANLAGSPAIKKGILQTVKVVDELVKV | |
| | | MGRHKPENIVIEMARENQTTQKGQKNSRERMKRIEEGIKELGSQILKE | |
| | | HPVENTQLQNEKLYLYYLQNGRDMYVDQELDINRLSDYDVDHIVPQSF | |
| | | LKDDSIDNKVLTRSDKNRGKSDNVPSEEVVKKMKNYWRQLLNAKLITQ | |
| | | RKFDNLTKAERGGLSELDKAGFIKRQLVETRQITKHVAQILDSRMNTK | |
| | | YDENDKLIREVKVITLKSKLVSDFRKDFQFYKVREINNYHHAHDAYLN | |
| | | AVVGTALIKKYPKLESEFVYGDYKVYDVRKMIAKSEQEIGSSGSETPG | |
| | | TSESATPESSGSEVEFSHEYWMRHALTLAKRARDEREVPVGAVLVLNN | |
| | | RVIGEGWNRAIGLHDPTAHAEIMALRQGGLVMQNYRLYDATLYSTFEP | |
| | | CVMCAGAMIHSRIGRVVFGVRNAKTGAAGSLMDVLHHPGMNHRVEITE | |
| | | GILADECAALLCRFFRMPRRVFNAQKKAQSSTDGKATAKYFFYSNIMN | |
| | | FFKTEITLANGEIRKRPLIETNGETGEIVWDKGRDFATVRKVLSMPQV | |
| | | NIVKKTEVQTGGFSKESILPKRNSDKLIARKKDWDPKKYGGFDSPTVA | |
| | | YSVLVVAKVEKGKSKKLKSVKELLGITIMERSSFEKNPIDFLEAKGYK | |
| | | EVKKDLIIKLPKYSLFELENGRKRMLASAGELQKGNELALPSKYVNFL | |
| | | YLASHYEKLKGSPEDNEQKQLFVEQHKHYLDEIIEQISEFSKRVILAD | |
| | | ANLDKVLSAYNKHRDKPIREQAENIIHLFTLTNLGAPAAFKYFDTTID | |
| | | RKRYTSTKEVLDATLIHQSITGLYETRIDLSQLGGDEGADKRTADGSE | |
| | | FESPKKKRKV | |

### Nucleobase Editors

Useful in the methods and compositions described herein are nucleobase editors that edit, modify or alter a target nucleotide sequence of a polynucleotide. Nucleobase editors described herein typically include a polynucleotide programmable nucleotide binding domain and a nucleobase editing domain (e.g., adenosine deaminase, cytidine deaminase, or a dual deaminase). A polynucleotide programmable nucleotide binding domain, when in conjunction with a bound guide polynucleotide (e.g., gRNA), can specifically bind to a target polynucleotide sequence and thereby localize the base editor to the target nucleic acid sequence desired to be edited.

### Polynucleotide Programmable Nucleotide Binding Domain

Polynucleotide programmable nucleotide binding domains bind polynucleotides (e.g., RNA, DNA). A polynucleotide programmable nucleotide binding domain of a base editor can itself comprise one or more domains (e.g., one or more nuclease domains). In some embodiments, the nuclease domain of a polynucleotide programmable nucleotide binding domain comprises an endonuclease or an exonuclease.

Disclosed herein are base editors comprising a polynucleotide programmable nucleotide binding domain comprising all or a portion (e.g., a functional portion) of a CRISPR protein (i.e., a base editor comprising as a domain all or a portion (e.g., a functional portion) of a CRISPR protein (e.g., a Cas protein), also referred to as a “CRISPR protein-derived domain” of the base editor). A CRISPR protein-derived domain incorporated into a base editor can be modified compared to a wild-type or natural version of the CRISPR protein. A CRISPR protein-derived domain can comprise one or more mutations, insertions, deletions, rearrangements and/or recombinations relative to a wild-type or natural version of the CRISPR protein.

Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1 (e.g., SEQ ID NO: 232), Cas12c/C2c3, Cas12d/CasY, Cas12e/CasX, Cas12g, Cas12h, Cas12i, and Cas12j/CasΦ, CARF, DinG, Turbo Cas9 (i.e., an SpCas9 with the amino acid alterations Q844R, V842L, F846Y, L847M, and I852F), homologues thereof, or modified versions thereof. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect to a corresponding wild-type enzyme such that the mutated CRISPR enzyme lacks the ability to cleave one or both strands of a target polynucleotide containing a target sequence can be used. A Cas protein (e.g., Cas9, Cas12) or a Cas domain (e.g., Cas9, Cas12) can refer to a polypeptide or domain with at least or at least about 50%, 60%, 70%, 80%, 90%, 91%, 92%, 93%, 94%, 95%, 96%, 97%, 98%, 99%, or 100% sequence identity and/or sequence homology to a wild-type exemplary Cas polypeptide or Cas domain. Cas (e.g., Cas9, Cas12) can refer to the wild-type or a modified form of the Cas protein that can comprise an amino acid change such as a deletion, insertion, substitution, variant, mutation, fusion, chimera, or any combination thereof. In some embodiments, a CRISPR protein-derived domain of a base editor can include all or a portion (e.g., a functional portion) of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

Some aspects of the disclosure provide high fidelity Cas9 domains. High fidelity Cas9 domains are known in the art and described, for example, in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each of which are incorporated herein by reference. An Exemplary high fidelity Cas9 domain is provided in the Sequence Listing as SEQ ID NO: 233.

In some embodiments, any of the Cas9 fusion proteins or complexes provided herein comprise one or more of a D10A, N497X, a R661X, a Q695X, and/or a Q926X mutation, or a corresponding mutation in any of the amino acid sequences provided herein, wherein X is any amino acid.

Typically, Cas9 proteins, such as Cas9 from S. pyogenes (spCas9), require a “protospacer adjacent motif (PAM)” or PAM-like motif, which is a 2-6 base pair DNA sequence immediately following the DNA sequence targeted by the Cas9 nuclease in the CRISPR bacterial adaptive immune system. The presence of an NGG PAM sequence is required to bind a particular nucleic acid region, where the “N” in “NGG” is adenosine (A), thymidine (T), or cytosine (C), and the G is guanosine. In some embodiments, any of the fusion proteins or complexes provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

In some embodiments, the napDNAbp is a circular permutant (e.g., SEQ ID NO: 238).

In some embodiments, the polynucleotide programmable nucleotide binding domain comprises a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). For example, where a polynucleotide programmable nucleotide binding domain comprises a nickase domain derived from Cas9, the Cas9-derived nickase domain can include a D10A mutation and a histidine at position 840. In another example, a Cas9-derived nickase domain comprises an H840A mutation, while the amino acid residue at position 10 remains a D.

In some embodiments, a Cas9 nuclease has an inactive (e.g., an inactivated) DNA cleavage domain, that is, the Cas9 is a nickase, referred to as an “nCas9” protein (for “nickase” Cas9; SEQ ID NO: 201). The Cas9 nickase may be a Cas9 protein that is capable of cleaving only one strand of a duplexed nucleic acid molecule (e.g., a duplexed DNA molecule). In some embodiments the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field and are within the scope of this disclosure.

Also provided herein are base editors comprising a polynucleotide programmable nucleotide binding domain which is catalytically dead (i.e., incapable of cleaving a target polynucleotide sequence). For example, in the case of a base editor comprising a Cas9 domain, the Cas9 can comprise both a D10A mutation and an H840A mutation. In further embodiments, a catalytically dead polynucleotide programmable nucleotide binding domain comprises a point mutation (e.g., D10A or H840A) as well as a deletion of all or a portion (e.g., a functional portion) of a nuclease domain. dCas9 domains are known in the art and described, for example, in Qi et al., “Repurposing CRISPR as an RNA-guided platform for sequence-specific control of gene expression.” Cell. 2013; 152 (5): 1173-83, the entire contents of which are incorporated herein by reference.

The term “protospacer adjacent motif (PAM)” or PAM-like motif refers to a 2-6 base pair DNA sequence immediately following the DNA sequence targeted by a nucleic acid programmable DNA binding protein. In some embodiments, the PAM can be a 5′ PAM (i.e., located upstream of the 5′ end of the protospacer). In other embodiments, the PAM can be a 3′ PAM (i.e., located downstream of the 5′ end of the protospacer). The PAM sequence can be any PAM sequence known in the art. Suitable PAM sequences include, but are not limited to, NGG, NGA, NGC, NGN, NGT, NGTT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A base editor provided herein can comprise a CRISPR protein-derived domain that is capable of binding a nucleotide sequence that contains a canonical or non-canonical protospacer adjacent motif (PAM) sequence.

In some embodiments, the PAM is an “NRN” PAM where the “N” in “NRN” is adenine (A), thymine (T), guanine (G), or cytosine (C), and the R is adenine (A) or guanine (G); or the PAM is an “NYN” PAM, wherein the “N” in NYN is adenine (A), thymine (T), guanine (G), or cytosine (C), and the Y is cytidine (C) or thymine (T), for example, as described in R. T. Walton et al., 2020, Science, 10.1126/science.aba8853 (2020), the entire contents of which are incorporated herein by reference.

Several PAM variants are described in Table 3 below.

TABLE 3
Cas9 proteins and corresponding PAM sequences.
N is A, C, T, or G; and V is A, C, or G.

| Variant | PAM |
|---|---|
| spCas9 | NGG |
| spCas9-VRQR | NGA |
| spCas9-VRER | NGCG |
| xCas9 (sp) | NGN |
| saCas9 | NNGRRT |
| saCas9-KKH | NNNRRT |
| spCas9-LRKIQK | NGTN |
| spCas9-LRVSQK | NGTN |
| spCas9-LRVSQL | NGTN |
| Cpf1 | 5′ (TTTV) |
| SpyMac | 5′-NAA-3′ |

In some embodiments, the PAM is NGC. In some embodiments, the NGC PAM is recognized by a Cas9 variant. In some embodiments, the Cas9 variant contains one or more amino acid substitutions selected from D1135V, G1218R, R1335Q, and T1337R (collectively termed VRQR) of spCas9 (SEQ ID No: 197), or a corresponding mutation in another Cas9. In some embodiments, the Cas9 variant contains one or more amino acid substitutions selected from D1135V, G1218R, R1335E, and T1337R (collectively termed VRER) of spCas9 (SEQ ID No: 197), or a corresponding mutation in another Cas9. In some embodiments, the Cas9 variant contains one or more amino acid substitutions selected from E782K, N968K, and R1015H (collectively termed KHH) of saCas9 (SEQ ID NO: 218).

In some cases, a Cas9 variant has specificity for the PAM 5′-NGC-3′. In some embodiments, a Cas9 variant includes one or more amino acid substitutions selected from D1135M, S1136Q, G1218K, E1219F, A1322R, D1332A, R1335E, and T1337K of spCas9 (SEQ ID No: 197), or a corresponding mutation in another Cas9. In some embodiments, a Cas9 variant includes one or more amino acid substitutions selected from D1135M, S1136Y, G1218K, E1219F, A1322R, D1332A, R1335E, and T1337K of spCas9 (SEQ ID No: 197), or a corresponding mutation in another Cas9. In some embodiments, the a Cas9 variant includes one or more amino acid substitutions selected from D1135L, S1136Y, G1218K, E1219F, A1322R, D1332A, R1335E, and T1337R of spCas9 (SEQ ID No: 197), or a corresponding mutation in another Cas9. In some embodiments, a Cas9 variant includes one or more amino acid substitutions selected from D1135M, S1136Y, G1218K, E1219F, A1322R, D1332A, R1335E, and T1337K of spCas9 (SEQ ID No: 197), or a corresponding mutation in another Cas9. In some embodiments, a Cas9 variant includes one or more amino acid substitutions selected from D1135L, S1136Y, G1218K, E1219F, A1283D, A1322R, D1332A, R1335E, and T1337K of spCas9 (SEQ ID No: 197), or a corresponding mutation in another Cas9. In some embodiments, a Cas9 variant includes one or more amino acid substitutions selected from D1135L, S1136Q, G1218K, E1219F, E1250K, A1283D, A1322R, D1332A, R1335E, and T1337K of spCas9 (SEQ ID No: 197), or a corresponding mutation in another Cas9. In some embodiments, a Cas9 variant includes one or more amino acid substitutions selected from D1135M, S1136Y, G1218K, E1219F, E1250K, A1283D, A1322R, D1332A, R1335E, and T1337R of spCas9 (SEQ ID No: 197), or a corresponding mutation in another Cas9. In some embodiments, a Cas9 variant includes one or more amino acid substitutions selected from R765A, Q768A, D1135L, S1136Y, G1218K, A1283D, E1219F, A1322R, D1332A, R1335E, and T1337K of spCas9 (SEQ ID No: 197), or a corresponding mutation in another Cas9. In some embodiments, any of the Cas9 proteins provided herein, including an SpCas9 comprises any one, two, three, four, five, six, seven, eight, nine, or ten of the following amino acid substitutions in a corresponding residue: R765A, Q768A, W1126R, R1359W, E1250K, A1239T, A1239V, A1283D, R1335D, D1135L, D1135M, D1135R, D1135W, S1136H, S1136Q, S1136Y, G1218D, G1218K, G1218R, G1218E, G1218L, E1219F, E1219K, E1219N, A1322A, A1322R, A1322K, D1332A, R1335V, T1337K, T1337T, D1332A, D1135V and T1337R.

In some embodiments, a CRISPR protein-derived domain of a base editor comprises all or a portion (e.g., a functional portion) of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); R. T. Walton et al. “Unconstrained genome targeting with near-PAMless engineered CRISPR-Cas9 variants” Science 10.1126/science.aba8853 (2020); Hu et al. “Evolved Cas9 variants with broad PAM compatibility and high DNA specificity,” Nature, 2018 Apr. 5, 556 (7699), 57-63; Miller et al., “Continuous evolution of SpCas9 variants compatible with non-G PAMs” Nat. Biotechnol., 2020 April; 38 (4): 471-481; the entire contents of each are hereby incorporated by reference.

Fusion Proteins or Complexes Comprising a NapDNAbp and a Cytidine Deaminase and/or Adenosine Deaminase

Some aspects of the disclosure provide fusion proteins or complexes comprising a Cas9 domain or other nucleic acid programmable DNA binding protein (e.g., Cas12) and one or more cytidine deaminase, adenosine deaminase, or cytidine adenosine deaminase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and/or adenosine deaminases provided herein. The domains of the base editors disclosed herein can be arranged in any order.

In some embodiments, the fusion proteins or complexes comprising a cytidine deaminase or adenosine deaminase and a napDNAbp (e.g., Cas9 or Cas12 domain) do not include a linker sequence. In some embodiments, a linker is present between the cytidine or adenosine deaminase and the napDNAbp. In some embodiments, cytidine or adenosine deaminase and the napDNAbp are fused via any of the linkers provided herein. For example, in some embodiments the cytidine or adenosine deaminase and the napDNAbp are fused via any of the linkers provided herein.

It should be appreciated that the fusion proteins or complexes of the present disclosure may comprise one or more additional features. For example, in some embodiments, the fusion protein or complex may comprise inhibitors, cytoplasmic localization sequences, export sequences, such as nuclear export sequences, or other localization sequences, as well as sequence tags that are useful for solubilization, purification, or detection of the fusion proteins or complexes. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FLASH tags, V5 tags, and SBP-tags. Additional suitable sequences will be apparent to those of skill in the art. In some embodiments, the fusion protein or complex comprises one or more His tags.

Exemplary, yet nonlimiting, fusion proteins are described in International PCT Application Nos. PCT/US2017/045381, PCT/US2019/044935, and PCT/US2020/016288, each of which is incorporated herein by reference for its entirety.

Fusion Proteins or Complexes with Internal Insertions

Provided herein are fusion proteins or complexes comprising a heterologous polypeptide fused to a nucleic acid programmable nucleic acid binding protein, for example, a napDNAbp. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is a deaminase (e.g., cytidine or adenosine deaminase) or a functional fragment thereof. For example, a fusion protein can comprise a deaminase flanked by an N-terminal fragment and a C-terminal fragment of a Cas9 or Cas12 (e.g., Cas12b/C2c1), polypeptide.

The deaminase can be a circular permutant deaminase. In some embodiments, the deaminase is a circular permutant TadA, circularly permutated at amino acid residue 116, 136, or 65 as numbered in a TadA reference sequence.

The fusion protein or complexes can comprise more than one deaminase. The fusion protein or complex can comprise, for example, 1, 2, 3, 4, 5 or more deaminases. The deaminases in a fusion protein or complex can be adenosine deaminases, cytidine deaminases, or a combination thereof.

In some embodiments, the napDNAbp in the fusion protein or complex contains a Cas9 polypeptide or a fragment thereof. The Cas9 polypeptide can be a variant Cas9 polypeptide. The Cas9 polypeptide can be a circularly permuted Cas9 protein.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase (dual deaminase)) can be inserted into a napDNAbp without compromising function of the deaminase (e.g., base editing activity) or the napDNAbp (e.g., ability to bind to target nucleic acid and guide nucleic acid).

In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in SEQ ID NO: 197. Cas9 polypeptide regions comprising a higher than average B-factor can include, for example, residues 792-872, 792-906, and 2-791 as numbered in SEQ ID NO: 197.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in SEQ ID NO: 197, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in SEQ ID NO: 197, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in SEQ ID NO: 197, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. Exemplary internal fusions base editors are provided in Table 4A below:

TABLE 4A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix truncated 165-end | ISLAY21 |
| IBE029 | TadA-Circular Permutant116 ins1067 | ISLAY29 |
| IBE031 | TadA-Circular Permutant 136 ins1248 | ISLAY31 |
| IBE032 | TadA-Circular Permutant 136ins 1052 | ISLAY32 |
| IBE035 | delta 792-872 TadA ins | ISLAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

A fusion protein can comprise a linker between the deaminase and the napDNAbp polypeptide. The linker can be a peptide or a non-peptide linker. For example, the linker can be an XTEN, (GGGS)ₙ (SEQ ID NO: 246), SGGSSGGS (SEQ ID NO: 330), (GGGGS)ₙ (SEQ ID NO: 247), (G)ₙ, (EAAAK)ₙ (SEQ ID NO: 248), (GGS)ₙ, SGSETPGTSESATPES (SEQ ID NO: 249). In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase but does not comprise a linker between the C-terminal Cas9 fragment and the deaminase. In some embodiments, the fusion protein comprises a linker between the C-terminal Cas9 fragment and the deaminase but does not comprise a linker between the N-terminal Cas9 fragment and the deaminase.

In some embodiments, the napDNAbp in the fusion protein or complex is a Cas12 polypeptide, e.g., Cas12b/C2c1, or a functional fragment thereof capable of associating with a nucleic acid (e.g., a gRNA) that guides the Cas 12 to a specific nucleic acid sequence. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS (SEQ ID NO: 250) or GSSGSETPGTSESATPESSG (SEQ ID NO: 251). In other embodiments, the linker is a rigid linker. In other embodiments of the above aspects, the linker is encoded by GGAGGCTCTGGAGGAAGC (SEQ ID NO: 252) or GGCTCTTCTGGATCTGAAACACCTGGCACAAGCGAGAGCGCCACCCCTGAGAGCTCTGGC (SEQ ID NO: 253).

In other embodiments, the fusion protein or complex contains a nuclear localization signal (e.g., a bipartite nuclear localization signal). In other embodiments, the amino acid sequence of the nuclear localization signal is MAPKKKRKVGIHGVPAA (SEQ ID NO: 261). In other embodiments of the above aspects, the nuclear localization signal is encoded by the following sequence:

ATGGCCCCAAAGAAGAAGCGGAAGGTCGGTATCCACGGAGTCCCAGCAGCC (SEQ ID NO: 262). In other embodiments, the Cas12b polypeptide contains a mutation that silences the catalytic activity of a RuvC domain. In other embodiments, the Cas12b polypeptide contains D574A, D829A and/or D952A mutations.

In some embodiments, the fusion protein or complex comprises a napDNAbp domain (e.g., Cas12-derived domain) with an internally fused nucleobase editing domain (e.g., all or a portion (e.g., a functional portion) of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 4B below.

TABLE 4B
Insertion loci in Cas12b proteins

| | Insertion site | Inserted between aa |
|---|---|---|
| BhCas12b | | |
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | | |
| position 1 | 147 | PD |
| position 2 | 248 | GG |
| position 3 | 299 | PE |
| position 4 | 991 | GE |
| position 5 | 1031 | KM |
| AaCas12b | | |
| position 1 | 157 | PG |
| position 2 | 258 | VG |
| position 3 | 310 | DP |
| position 4 | 1008 | GE |
| position 5 | 1044 | GK |

In some embodiments, the base editing system described herein is an ABE with TadA inserted into a Cas9. Polypeptide sequences of relevant ABEs with TadA inserted into a Cas9 are provided in the attached Sequence Listing as SEQ ID NOs: 263-308.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### A to G Editing

In some embodiments, a base editor described herein comprises an adenosine deaminase domain. Such an adenosine deaminase domain of a base editor can facilitate the editing of an adenine (A) nucleobase to a guanine (G) nucleobase by deaminating the A to form inosine (I), which exhibits base pairing properties of G. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In an embodiment an adenosine deaminase domain of a base editor comprises all or a portion (e.g., a functional portion) of an ADAT comprising one or more mutations which permit the ADAT to deaminate a target A in DNA. For example, the base editor can comprise all or a portion (e.g., a functional portion) of an ADAT from Escherichia coli (EcTadA) comprising one or more of the following mutations: D108N, A106V, D147Y, E155V, L84F, H123Y, I156F, or a corresponding mutation in another adenosine deaminase. Exemplary ADAT homolog polypeptide sequences are provided in the Sequence Listing as SEQ ID NOs: 1 and 309-315.

The adenosine deaminase can be derived from any suitable organism (e.g., E. coli). In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that correspond to any of the mutations described herein (e.g., any of the mutations identified in ecTadA) can be generated accordingly.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identify plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein.

It should be appreciated that any of the mutations provided herein (e.g., based on a TadA reference sequence, such as TadA*7.10 (SEQ ID NO: 1)) can be introduced into other adenosine deaminases, such as E. coli TadA (ecTadA), S. aureus TadA (saTadA), or other adenosine deaminases (e.g., bacterial adenosine deaminases). In some embodiments, the TadA reference sequence is TadA*7.10 (SEQ ID NO: 1). It would be apparent to the skilled artisan that additional deaminases may similarly be aligned to identify homologous amino acid residues that can be mutated as provided herein. Thus, any of the mutations identified in a TadA reference 5 sequence can be made in other adenosine deaminases (e.g., ecTada) that have homologous amino acid residues. It should also be appreciated that any of the mutations provided herein can be made individually or in any combination in a TadA reference sequence or another adenosine deaminase.

In some embodiments, the adenosine deaminase comprises an alteration or set of alterations selected from those listed in Tables 5A-5E below:

TABLE 5A
Adenosine Deaminase Variants. Residue positions in the E. coli TadA variant (TadA*) are indicated.

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| TadA*0.1 | W | R | H | N | P | | R | N | L | S | A | D | H | G | A | S | D | R | E | I | K | K |
| TadA*0.2 | W | R | H | N | P | | R | N | L | S | A | D | H | G | A | S | D | R | E | I | K | K |
| TadA*1.1 | W | R | H | N | P | | R | N | L | S | A | N | H | G | A | S | D | R | E | I | K | K |
| TadA*1.2 | W | R | H | N | P | | R | N | L | S | V | N | H | G | A | S | D | R | E | I | K | K |
| TadA*2.1 | W | R | H | N | P | | R | N | L | S | V | N | H | G | A | S | Y | R | V | I | K | K |
| TadA*2.2 | W | R | H | N | P | | R | N | L | S | V | N | H | G | A | S | Y | R | V | I | K | K |
| TadA*2.3 | W | R | H | N | P | | R | N | L | S | V | N | H | G | A | S | Y | R | V | I | K | K |
| TadA*2.4 | W | R | H | N | P | | R | N | L | S | V | N | H | G | A | S | Y | R | V | I | K | K |
| TadA*2.5 | W | R | H | N | P | | R | N | L | S | V | N | H | G | A | S | Y | R | V | I | K | K |
| TadA*2.6 | W | R | H | N | P | | R | N | L | S | V | N | H | G | A | S | Y | R | V | I | K | K |
| TadA*2.7 | W | R | H | N | P | | R | N | L | S | V | N | H | G | A | S | Y | R | V | T | K | K |
| TadA*2.8 | W | R | H | N | P | | R | N | L | S | V | N | H | G | A | S | Y | R | V | I | K | K |
| TadA*2.9 | W | R | H | N | P | | R | N | L | S | V | N | H | G | A | S | Y | R | V | I | K | K |
| TadA*2.10 | W | R | H | N | P | | R | N | L | S | V | N | H | G | A | S | Y | R | V | I | K | K |
| TadA*2.11 | W | R | H | N | P | | R | N | L | S | V | N | H | G | A | S | Y | R | V | I | K | K |
| TadA*2.12 | W | R | H | N | P | | R | N | L | S | V | N | H | G | A | S | Y | R | V | I | K | K |
| TadA*3.1 | W | R | H | N | P | | R | N | F | S | V | N | Y | G | A | S | Y | R | V | F | K | K |
| TadA*3.2 | W | R | H | N | P | | R | N | F | S | V | N | Y | G | A | S | Y | R | V | F | K | K |
| TadA*3.3 | W | R | H | N | P | | R | N | F | S | V | N | Y | G | A | S | Y | R | V | F | K | K |
| TadA*3.4 | W | R | H | N | P | | R | N | F | S | V | N | Y | G | A | S | Y | R | V | F | K | K |
| TadA*3.5 | W | R | H | N | P | | R | N | F | S | V | N | Y | G | A | S | Y | R | V | F | K | K |
| TadA*3.6 | W | R | H | N | P | | R | N | F | S | V | N | Y | G | A | S | Y | R | V | F | K | K |
| TadA*3.7 | W | R | H | N | P | | R | N | F | S | V | N | Y | G | A | S | Y | R | V | F | K | K |
| TadA*3.8 | W | R | H | N | P | | R | N | F | S | V | N | Y | G | A | S | Y | R | V | F | K | K |
| TadA*4.1 | W | R | H | N | P | | R | N | L | S | V | N | H | G | N | S | Y | R | V | I | K | K |
| TadA*4.2 | W | G | H | N | P | | R | N | L | S | V | N | H | G | N | S | Y | R | V | I | K | K |
| TadA*4.3 | W | R | H | N | P | | R | N | F | S | V | N | Y | G | N | S | Y | R | V | F | K | K |
| TadA*5.1 | W | R | L | N | P | | L | N | F | S | V | N | Y | G | A | C | Y | R | V | F | N | K |
| TadA*5.2 | W | R | H | S | P | | R | N | F | S | V | N | Y | G | A | S | Y | R | V | F | K | T |
| TadA*5.3 | W | R | L | N | P | | L | N | I | S | V | N | Y | G | A | C | Y | R | V | F | N | K |
| TadA*5.4 | W | R | H | S | P | | R | N | F | S | V | N | Y | G | A | S | Y | R | V | F | K | T |
| TadA*5.5 | W | R | L | N | P | | L | N | F | S | V | N | Y | G | A | C | Y | R | V | F | N | K |
| TadA*5.6 | W | R | L | N | P | | L | N | F | S | V | N | Y | G | A | C | Y | R | V | F | N | K |
| TadA*5.7 | W | R | L | N | P | | L | N | F | S | V | N | Y | G | A | C | Y | R | V | F | N | K |
| TadA*5.8 | W | R | L | N | P | | L | N | F | S | V | N | Y | G | A | C | Y | R | V | F | N | K |
| TadA*5.9 | W | R | L | N | P | | L | N | F | S | V | N | Y | G | A | C | Y | R | V | F | N | K |
| TadA*5.10 | W | R | L | N | P | | L | N | F | S | V | N | Y | G | A | C | Y | R | V | F | N | K |
| TadA*5.11 | W | R | L | N | P | | L | N | F | S | V | N | Y | G | A | C | Y | R | V | F | N | K |
| TadA*5.12 | W | R | L | N | P | | L | N | F | S | V | N | Y | G | A | C | Y | R | V | F | N | K |
| TadA*5.13 | W | R | H | N | P | | L | D | F | S | V | N | Y | A | A | S | Y | R | V | F | K | K |
| TadA*5.14 | W | R | H | N | S | | L | N | F | C | V | N | Y | G | A | S | Y | R | V | F | K | K |
| TadA*6.1 | W | R | H | N | S | | L | N | F | S | V | N | Y | G | N | S | Y | R | V | F | K | K |
| TadA*6.2 | W | R | H | N | T | V | L | N | F | S | V | N | Y | G | N | S | Y | R | V | F | N | K |
| TadA*6.3 | W | R | L | N | S | | L | N | F | S | V | N | Y | G | A | C | Y | R | V | F | N | K |
| TadA*6.4 | W | R | L | N | S | | L | N | F | S | V | N | Y | G | N | C | Y | R | V | F | N | K |
| TadA*6.5 | W | R | L | N | T | V | L | N | F | S | V | N | Y | G | A | C | Y | R | V | F | N | K |
| TadA*6.6 | W | R | L | N | T | V | L | N | F | S | V | N | Y | G | N | C | Y | R | V | F | N | K |
| TadA*7.1 | W | R | L | N | A | | L | N | F | S | V | N | Y | G | A | C | Y | R | V | F | N | K |
| TadA*7.2 | W | R | L | N | A | | L | N | F | S | V | N | Y | G | N | C | Y | R | V | F | N | K |
| TadA*7.3 | L | R | L | N | A | | L | N | F | S | V | N | Y | G | A | C | Y | R | V | F | N | K |
| TadA*7.4 | R | R | L | N | A | | L | N | F | S | V | N | Y | G | A | C | Y | R | V | F | N | K |
| TadA*7.5 | W | R | L | N | A | | L | N | F | S | V | N | Y | G | A | C | Y | H | V | F | N | K |
| TadA*7.6 | W | R | L | N | A | | L | N | I | S | V | N | Y | G | A | C | Y | P | V | F | N | K |
| TadA*7.7 | L | R | L | N | A | | L | N | F | S | V | N | Y | G | A | C | Y | P | V | F | N | K |
| TadA*7.8 | L | R | L | N | A | | L | N | F | S | V | N | Y | G | N | C | Y | R | V | F | N | K |
| TadA*7.9 | L | R | L | N | A | | L | N | F | S | V | N | Y | G | N | C | Y | P | V | F | N | K |
| TadA*7.10 | R | R | L | N | A | | L | N | F | S | V | N | Y | G | A | C | Y | P | V | F | N | K |

TABLE 5B
TadA*8 Adenosine Deaminase Variants. Residue positions in the E. coli TadA variant
(TadA*) are indicated. Alterations are referenced to TadA*7.10 (first row).

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| TadA*7.10 | R | L | A | L | I | V | F | V | N | Y | C | Y | P | Q | V | F | N | T |
| TadA*8.1 | | | | | | | | | | | | T | | | | | | |
| TadA*8.2 | | | | | | | | | | | | R | | | | | | |
| TadA*8.3 | | | | | | | | | | | | | | S | | | | |
| TadA*8.4 | | | | | | | | | | H | | | | | | | | |
| TadA*8.5 | | | | | | S | | | | | | | | | | | | |
| TadA*8.6 | | | | | | | | | | | | | | | | | | R |
| TadA*8.7 | | | | | | | | | | | | | | R | | | | |
| TadA*8.8 | | | | | | | | | | H | | R | | R | | | | |
| TadA*8.9 | | | | | Y | | | | | | | R | | R | | | | |
| TadA*8.10 | | | | | | | | | | | | R | | R | | | | R |
| TadA*8.11 | | | | | | | | | | | | T | | R | | | | |
| TadA*8.12 | | | | | | | | | | | | T | | S | | | | |
| TadA*8.13 | | | | | Y | | | | | H | | R | | R | | | | |
| TadA*8.14 | | | | | Y | S | | | | | | | | | | | | |
| TadA*8.15 | | | | | | S | | | | | | R | | | | | | |
| TadA*8.16 | | | | | | S | | | | H | | R | | | | | | |
| TadA*8.17 | | | | | | S | | | | | | | | R | | | | |
| TadA*8.18 | | | | | | S | | | | H | | | | R | | | | |
| TadA*8.19 | | | | | | S | | | | H | | R | | R | | | | |
| TadA*8.20 | | | | | Y | S | | | | H | | R | | R | | | | |
| TadA*8.21 | | | | | | | | | | | | R | | S | | | | |
| TadA*8.22 | | | | | | S | | | | | | | | S | | | | |
| TadA*8.23 | | | | | | S | | | | H | | | | | | | | |
| TadA*8.24 | | | | | | S | | | | H | | T | | | | | | |

TABLE 5C
TadA*9 Adenosine Deaminase Variants.

| TadA*9 Description | Alterations |
|---|---|
| TadA*9.1 | E25F, V82S, Y123H, T133K, Y147R, Q154R |
| TadA*9.2 | E25F, V82S, Y123H, Y147R, Q154R |
| TadA*9.3 | V82S, Y123H, P124W, Y147R, Q154R |
| TadA*9.4 | L51W, V82S, Y123H, C146R, Y147R, Q154R |
| TadA*9.5 | P54C, V82S, Y123H, Y147R, Q154R |
| TadA*9.6 | Y73S, V82S, Y123H, Y147R, Q154R |
| TadA*9.7 | N38G, V82T, Y123H, Y147R, Q154R |
| TadA*9.8 | R23H, V82S, Y123H, Y147R, Q154R |
| TadA*9.9 | R21N, V82S, Y123H, Y147R, Q154R |
| TadA*9.10 | V82S, Y123H, Y147R, Q154R, A158K |
| TadA*9.11 | N72K, V82S, Y123H, D139L, Y147R, Q154R, |
| TadA*9.12 | E25F, V82S, Y123H, D139M, Y147R, Q154R |
| TadA*9.13 | M70V, V82S, M94V, Y123H, Y147R, Q154R |
| TadA*9.14 | Q71M, V82S, Y123H, Y147R, Q154R |
| TadA*9.15 | E25F, V82S, Y123H, T133K, Y147R, Q154R |
| TadA*9.16 | E25F, V82S, Y123H, Y147R, Q154R |
| TadA*9.17 | V82S, Y123H, P124W, Y147R, Q154R |
| TadA*9.18 | L51W, V82S, Y123H, C146R, Y147R, Q154R |
| TadA*9.19 | P54C, V82S, Y123H, Y147R, Q154R |
| TadA*9.2 | Y73S, V82S, Y123H, Y147R, Q154R |
| TadA*9.21 | N38G, V82T, Y123H, Y147R, Q154R |
| TadA*9.22 | R23H, V82S, Y123H, Y147R, Q154R |
| TadA*9.23 | R21N, V82S, Y123H, Y147R, Q154R |
| TadA*9.24 | V82S, Y123H, Y147R, Q154R, A158K |
| TadA*9.25 | N72K, V82S, Y123H, D139L, Y147R, Q154R, |
| TadA*9.26 | E25F, V82S, Y123H, D139M, Y147R, Q154R |
| TadA*9.27 | M70V, V82S, M94V, Y123H, Y147R, Q154R |
| TadA*9.28 | Q71M, V82S, Y123H, Y147R, Q154R |
| TadA*9.29 | E25F, I76Y, V82S, Y123H, Y147R, Q154R |
| TadA*9.30 | I76Y, V82T, Y123H, Y147R, Q154R |
| TadA*9.31 | N38G, I76Y, V82S, Y123H, Y147R, Q154R |
| TadA*9.32 | N38G, I76Y, V82T, Y123H, Y147R, Q154R |
| TadA*9.33 | R23H, I76Y, V82S, Y123H, Y147R, Q154R |
| TadA*9.34 | P54C, I76Y, V82S, Y123H, Y147R, Q154R |
| TadA*9.35 | R21N, I76Y, V82S, Y123H, Y147R, Q154R |
| TadA*9.36 | I76Y, V82S, Y123H, D138M, Y147R, Q154R |
| TadA*9.37 | Y72S, I76Y, V82S, Y123H, Y147R, Q154R |
| TadA*9.38 | E25F, I76Y, V82S, Y123H, Y147R, Q154R |
| TadA*9.39 | I76Y, V82T, Y123H, Y147R, Q154R |
| TadA*9.40 | N38G, I76Y, V82S, Y123H, Y147R, Q154R |
| TadA*9.41 | N38G, I76Y, V82T, Y123H, Y147R, Q154R |
| TadA*9.42 | R23H, I76Y, V82S, Y123H, Y147R, Q154R |
| TadA*9.43 | P54C, I76Y, V82S, Y123H, Y147R, Q154R |
| TadA*9.44 | R21N, I76Y, V82S, Y123H, Y147R, Q154R |
| TadA*9.45 | I76Y, V82S, Y123H, D138M, Y147R, Q154R |
| TadA*9.46 | Y72S, I76Y, V82S, Y123H, Y147R, Q154R |
| TadA*9.47 | N72K, V82S, Y123H, Y147R, Q154R |
| TadA*9.48 | Q71M, V82S, Y123H, Y147R, Q154R |
| TadA*9.49 | M70V, V82S, M94V, Y123H, Y147R, Q154R |
| TadA*9.50 | V82S, Y123H, T133K, Y147R, Q154R |
| TadA*9.51 | V82S, Y123H, T133K, Y147R, Q154R, A158K |
| TadA*9.52 | M70V, Q71M, N72K, V82S, Y123H, Y147R, Q154R |
| TadA*9.53 | N72K, V82S, Y123H, Y147R, Q154R |
| TadA*9.54 | Q71M, V82S, Y123H, Y147R, Q154R |
| TadA*9.55 | M70V, V82S, M94V, Y123H, Y147R, Q154R |
| TadA*9.56 | V82S, Y123H, T133K, Y147R, Q154R |
| TadA*9.57 | V82S, Y123H, T133K, Y147R, Q154R, A158K |
| TadA*9.58 | M70V, Q71M, N72K, V82S, Y123H, Y147R, Q154R |

Alterations are referenced to TadA*7.10. Additional details of TadA*9 adenosine deaminases are described in International PCT Application No. PCT/US2020/049975, which is incorporated herein by reference in its entirety for all purposes.

In some embodiments, the adenosine deaminase comprises a TadA*8.20 adenosine deaminase variant further comprising an F149Y amino acid alteration. In some embodiments, the adenosine deaminase comprises a TadA*8.20 adenosine deaminase variant further comprising the amino acid alterations R147D, F149Y, T166I, and D167N (TadA*8.10+). In some embodiments, the adenosine deaminase comprises a TadA*8.20 adenosine deaminase variant further comprising the amino acid alterations S82T and F149Y (TadA*9v1). In some embodiments, the adenosine deaminase comprises a TadA*8.20 adenosine deaminase variant further comprising the amino acid alterations Y147D, F149Y, T166I, D167N and S82T (TadA*9v2).

In some embodiments, the adenosine deaminase comprises one or more of M1I, M1S, S2A, S2E, S2H, S2R, S2L, E3L, V4D, V4E, V4M, V4K, V4S, V4T, V4A, E5K, F6S, F6G, F6H, F6Y, F6I, F6E, S7K, H8E, H8Y, H8H, H8Q, H8E, H8G, H8S, E9Y, E9K, E9V, E9E, Y10F, Y10W, Y10Y, M12S, M12L, M12R, M12W, R13H, R13I, R13Y, R13R, R13G, R13S, H14N, A15D, A15V, A15L, A15H, T17T, T17A, T17W, T17L, T17F, T17R, T17S, L18A, L18E, L18N, L18L, L18S, A19N, A19H, A19K, A19A, A19D, A19G, A19M, R21N, K20K, K20A, K20R, K20E, K20G, K20C, K20Q R21A, R21R, R21N, R21Y, R21C G22P, A22W, A22R, W23D, R23H, W23G, W23Q, W23L, W23R, W23H W23D W23M, W23W, W23I, D24E, D24G, D24W, D24D, D24R, E25F, E25M, E25D, E25A, E25G, E25R, E25E, E25H E25V, E25S, E25Y, R26D, R26E, R26G, R26N, R26Q, R26C, R26L, R26K, R26W, R26C, R26P, R26R, R26A, R26H, E27E, E27Q, E27H, E27C, E27G, E27K, E27S, E27P, E27R, E27L, E27V, E27D, V28V, V28A, V28C, V28G, V28P, V28S, V28T, P29V, P29P, P29A, P29G, P29K, P29L, V30V, V30I, V30L, V30F, V30G, V30A, V30M, L34S, L34V, L34L, L34M, L34W, L34G, H36E, H36V, L36H, H36L, H36N, N37N, N37H, N37R, N37T, N37S, N38G, N38R, N38N, N38E, V40I, W45A, W45W, W45R, W45L, W45N, N46N, N46M, N46P, N46G, N46L, N46R, N46V, R46W, R46F, R46Q, R46M, R47A, R47Q, R47F, R47K, R47P, R47W, R47M, R47R, R47G, R47S, R47V, R47H, P48T, P48L, P48A, P48I, P48S, P48R, P48K, P48D, P48E, P48H, P48G, P48P, P48N, I49G, I49H, I49V, I49F, I49H, I49I, I49M, I49N, I49K, I49Q, I49T, G50L, G50S, G50R, G50G, R51H, R51L, R51N, L51W, R51Y, R51G, R51V, R51R, H52D, H52Y, H521, H52H, D53D, D53E, D53G, D53P, P54C, P54T, P54P, P54E, A55H, T55A, T55I, T55V, T55G, T55T, A56A, A56H, A56W, A56E, A56S, H57P, H57A, H57H, H57N, A58G, A58E, A58A, A58R, E59A, E59G, E59I, E59Q, E59W, E59E, E59T, E59H, E59P, M61A, M61I, M61L, M61V, M61P, M61G, M61I, L63S, L63V, L63T, L63R, L63H, L63A, R64A, R64Q, R64R, R64D, Q65V, Q65H, Q65G, Q65P, Q65F, Q65Q, Q65R, G66V, G66E, G66T, G66G, G66C, G67G, G67W, G67I, G67A, G67D, G67L, G67V, L68Q, L68M, L68V, L68H, L68L, L68G, V69A, V69M, V69V, M70V, M70L, E70A, M70A, M70M, M70E, M70T, M70v, Q71M, Q71N, Q71L, Q71R, Q71Q, Q71I, N72A, N72K, N72S, N72D, N72Y, N72N, N72H, N72G, N72M, Y73G, Y73I, Y73K, Y73R, Y73S, Y73Y, Y73H, Y73A, R74A, R74Q, R74G, R74K, R74L, R74N, R74G, R74K, R74R, I76H, I76R, I76W, I76Y, I76V, I76Q, I76L, I76D, I76F, I76I, I76N, I76T, I76Y, D77G, D77D, D77A, D77Q, A78Y, A78T, A78G, A78A, A78I, T79M, T79R, T79L, T79T, L80M, L80Y, L80I, L80V, L80L, Y81D, Y81V, Y81Y, Y81M, V82A, V82S, V82G, V82T, V82V, V82Q, V82Y, T83L, T83F, T83T, T83N, L84E, L84F, L84Y, L84I, L84L, L84M, L84A, L84T, L84S, E85K, E85G, E85P, E85S, E85E, E85F, E85V, E85R, P86T, P86C, P86P, P86L, P86N, P86K, P86H, C87M, C87I, C87S, C87N, C87P, S87C, S87L, S87V, V88A, V88M, V88V, V88T, V88E, V88D, V88S, C90S, C90P, C90A, C90T, C90M, A91A, A91G, A91S, A91V, A91T, A91C, A91L, G92T, G92M, G92A, G92Y, G92G, A93I, A93C, A93M, A93V, A93A, M94M, M94T, M94A, M94V, M94L, M94I, M94H, 195S, 195G, 195L, 195H, 195V, H96A, H96L, H96R, H96S, H96H, H96N, H96E, S97C, S97G, S97I, S97M, S97R, S97S, S97P, R98K, R981, R98N, R98Q, R98G, R98H, R98C, R98L, R98R, G100R, G100V, G100K, G100A, G100S, G100M, G100I, R101V, R101R, R101S, R101C, V102A, V102F, V102I, V102V, D103A, V103A, V103G, V103F, V103V, F104G, D104N, F104V, F104I, F104L, F104A, F104F, F104R, G105V, G105W, G105G, G105M, G105A, A106T, V106Q, V106F, V106W, V106M, A106A, A106Q, A106F, A106G, A106W, A106M, A106V, A106R, A106L, A106S, A106B, A106I, R107C, R107G, R107P, R107K, R107A, R107N, R107W, R107H, R107S, R107R, R107F, D108N, D108F, D108G, D108V, D108A, D108Y, D108H, D108I, D108K, D108L, D108M, D108Q, N108Q, N108F, N108W, N108M, N108K, D108K, D108F, D108M, D108Q, D108R, D108W, D108S, D108E, D108T, D108R, D108D, A109H, A109K, A109R, A109S, A109T, A109V, A109A, A109D, K110G, K110H, K110I, K110R, K110T, K110K, K110A, K110I, T111A, T111G, T111H, T111R, T111T, T111K, G112A, G112G, G112H, G112T, G112R, A113N, A114G, A114H, A114V, A114C, A114S, A114A, G115S, G115G, G115M, G115L, G115A, G115F, L117M, L117L, L117W, L117A, L117S, L117N, L117V, M118D, M118G, M118K, M118N, M118V, M118M, M118L, M118R, D119L, D119N, D119S, D119V, D119D, V120H, V120L, V120V, V120T, V120A, V120E, V120G, V120D, L121D, L121M, L121N, L121K, L121L, H122H, H122N, H122P, H122R, H122S, H122Y, H122G, H122T, H122L, H123C, H123G, H123P, H123V, H123Y, Y123H, H123Y, H123H, P124P, P124H, P124A, P124Y, P124D, P124G, P124I, P124L, P124W, G125H, G125I, G125A, G125M, G125K, G125G, G125P, M126D, M126H, M126K, M126I, M126N, M1260, M126S, M126Y, M126M, M126G, N127H, N127S, N127D, N127K, N127R, N127N, N127I, N127P, N127M, H128R, H128N, H128L, H128H, R129H, R129Q, R129V, R129I, R129E, R129V, R129R, R129M, R129P, V130R, V130V, V130E, V130D, E131E, E131I, E131V, E131K, I132I, I132F, I132T, I132L, I132V, I132E, T133V, T133E, T133G, T133K, T133T, T133A, T133H, T133F, T133I, E134A, E134E, E134G, E134I, E134H, E134K, E134T, G135G, G135V, G135I, G135P, G135E, I136G, I136L, I136T, I136I, 1137A, 1137D, 1137E, L137M, 1137S, L137L, L1371, A138D, A138E, A138G, S138A, A138N, A138S, A138T, A138V, A138Y, A138A, A138M, A138L, D139E, D139I, D139C, D139L, D139M, D139D, D139G, D139H, D139A, E140A, E140C, E140L, E140R, E140K, E140E, E140D, C141S, C141A, C141C, C141V, C141E, A142N, A142D, A142G, A142A, A142L, A142S, A142T, A142N, A142S, A142V, A142E, A142C, A143D, A143E, A143G, A143D, A143G, A143E, A143L, A143W, A143M, A143S, A143Q, A143R, A143A, A143I, L144S, L144L, L144T, L144A, L145A, L145F, L145G, L145D, L145L, L145C, L145E, L145s, C146R, S146A, S146C, S146D, S146F, S146R, S146T, S146D, S146G, S146S, S146L, D147D, D147L, D147F, D147G, D147Y, Y147T, Y147R, Y147D, D147R, D147Y, D147A, D147T, D147H, D147F, D147U, D147V, D1471, D147C, F148L, F148F, F148R, F148Y, F148A, F148T, F149C, F149M, F149R, F149Y, F149N, F149F, F149A, F149T, F149V R150R, R150M, R150D, R150F, M151F, M151P, M151R, M151V, M151M, M151E, R152C, R152F, R152H, R152P, R152R, R152P, R152Q, R152M, R1520, R153C, R153Q, R153R, R153V, R153E, R153A, R153P, Q154E, Q154H, Q154M, Q154R, Q154L, Q154S, Q154V, Q154Q, Q154F, Q154I, Q154A, Q154K, E155F, E155G, E155I, E155K, E155P, E155V, E155D, E155E, E155L, E155Q, I156V, I156A, I156I, I156L, I156F, I156D, I156K, I156N, I156R, I156Y, E157A, E157F, E157I, E157P, E157T, E157V, N157K, K157N, K157V, K157P, K157I, K157F, K157F, K157T, K157A, K157S, K157R, A158Q, A158K, A158V, A158A, A158D, A158S, A158T, A158N, Q159S, Q159Q, Q159A, Q159F, Q159K, Q159L, Q159N, K160A, K160S, K160E, K160K, K160N, K160F, K160Q, K161T, K161K, K161R, K161I, K161A, K161N, K161Q, K161S, K161T, A162D, A162Q, R162H, R162P, A162S, A162A, A162N, A162M, A162K, Q163G, Q163S, Q163Q, Q163A, Q163H, Q163N, Q163R, S164F, S164S, S164Q, S164I, S164R, S164Y, S165S, S165P, S165Q, S165A, S165D, S165I, S165T, S165Y, T166T, T166Q, T166E, T166S, T166D, T166K, T166I, T166N, T166P, T166R, D167S D167D, D167I, D167G, D167T, D167A and/or D167N mutation in a TadA reference sequence (e.g., TadA*7.10,ecTadA, or TadA8e), and any alternative mutation at the corresponding position, or one or more corresponding mutations in another adenosine deaminase. Additional mutations are described in U.S. Patent Application Publication No. 2022/0307003 A1 U.S.

U.S. Pat. No. 11,155,803, and International Patent Application Publications No. WO 2023/288304 A2, PCT/CN2022/143408, WO 2018/027078 A1, WO 2021/158921 A1 and WO 2023/034959 A2, the disclosures of which are incorporated herein by reference in their entirety for all purposes.

In various embodiments, an adenosine deaminase of the disclosure lacks an N-terminal methionine.

In some embodiments, the disclosure provides TadA variants comprising an alteration at an amino acid selected from one or more of L36, 176, V82, Y147, Q154, and N157 compared to TadA*7.10. In some embodiments, the disclosure provides TadA variants comprising one or more of the following alterations relative to TadA*7.10: L36H, I76Y, V82T, Y147T, Q154S, and N157K. In some embodiments, the disclosure provides TadA variants comprising the following alterations relative to TadA*7.10: L36H, I76Y, V82T, Y147T, Q154S, and N157K. In some embodiments, the disclosure provides TadA variants comprising the following alterations relative to TadA*7.10: F84Y, A109L, A109V, A109I, A109F, A109S, A109T, A109N, V155S, V155T, V155N, F156Y, F156W, F156R, F156N, and F156Q. In some embodiments, the disclosure provides TadA variants comprising the following alterations relative to TadA*7.10: E3N, E3K, E3G, F6A, H14D, L18A, W23I, W23R, P29T, P29Y, P29Q, V35Q, L36S, N38D, G42M, N46Y, P48A, G50A, H52L, A62V, L63R, L63F, Q65R, G67N, L68V, M70I, N72Y, T79H, Y81V, V82S, M94R, G100V, V102E, V102S, R107A, A114C, G115E, M118L, D119L, H122T, P124H, P124K, P124Q, H128R, V130F, I132K, I132T, E140L, A142N, A142S, L144Q, L145R, L145N, Y147A, F149A, R152P, F156N, and K160E.

In some embodiments, the disclosure provides TadA variants comprising a V82T, Y147T, and/or a Q154S mutation. In some embodiments, the disclosure provides TadA variants comprising a V82T, Y147T, and/or a Q154S mutation. In some embodiments, the disclosure provides TadA*8.8 further comprising a V82T mutation. In some embodiments, the disclosure provides TadA*8.8 further comprising a V82T, a Y147T, and a Q154S mutation. In some embodiments, the disclosure provides TadA*8.17 further comprising a V82T mutation. In some embodiments, the disclosure provides TadA*8.17 further comprising a V82T, a Y147T, and a Q154S mutation. In some embodiments, the disclosure provides TadA*8.20 further comprising a V82T mutation. In some embodiments, the disclosure provides TadA*8.20 further comprising a V82T, a Y147T, and a Q154S mutation.

In embodiments, a variant of TadA*7.10 comprises one or more alterations selected from any of those alterations provided herein.

In particular embodiments, an adenosine deaminase heterodimer comprises a TadA*8 domain and an adenosine deaminase domain selected from Staphylococcus aureus (S. aureus) TadA, Bacillus subtilis (B. subtilis) TadA, Salmonella typhimurium (S. typhimurium) TadA, Shewanella putrefaciens (S. putrefaciens) TadA, Haemophilus influenzae F3031 (H. influenzae) TadA, Caulobacter crescentus (C. crescentus) TadA, Geobacter sulfurreducens (G. sulfurreducens) TadA, or TadA*7.10.

In some embodiments, the TadA*8 is a variant as shown in Table 5D. Table 5D shows certain amino acid position numbers in the TadA amino acid sequence and the amino acids present in those positions in the TadA-7.10 adenosine deaminase. Table 5D also shows amino acid changes in TadA variants relative to TadA-7.10 following phage-assisted non-continuous evolution (PANCE) and phage-assisted continuous evolution (PACE), as described in M. Richter et al., 2020, Nature Biotechnology, doi.org/10.1038/s41587-020-0453-z, the entire contents of which are incorporated by reference herein. In some embodiments, the TadA*8 is TadA*8a, TadA*8b, TadA*8c, TadA*8d, or TadA*8e. In some embodiments, the TadA*8 is TadA*8e. In one embodiment, an adenosine deaminase is a TadA*8 that comprises or consists essentially of SEQ ID NO: 316 or a fragment thereof having adenosine deaminase activity.

TABLE 5D
Select TadA*8 Variants

| | | TadA amino acid number | | | | | | | | | |
|---|---|---|---|---|---|---|---|---|---|---|---|
| | TadA | 26 | 88 | 109 | 111 | 119 | 122 | 147 | 149 | 166 | 167 |
| | TadA- | R | V | A | T | D | H | Y | F | T | D |
| | 7.10 | | | | | | | | | | |
| PANCE 1 | | | | | R | | | | | | |
| PANCE 2 | | | | S/T | R | | | | | | |
| PACE | TadA-8a | C | | S | R | N | N | D | Y | I | N |
| | TadA-8b | | A | S | R | N | N | | Y | I | N |
| | TadA-8c | C | | S | R | N | N | | Y | I | N |
| | TadA-8d | | A | | R | N | | | Y | | |
| | TadA-8e | | | S | R | N | N | D | Y | I | N |

In some embodiments, the TadA variant is a variant as shown in Table 5E. Table 5E 5 shows certain amino acid position numbers in the TadA amino acid sequence and the amino acids present in those positions in the TadA*7.10 adenosine deaminase. In some embodiments, the TadA variant is MSP605, MSP680, MSP823, MSP824, MSP825, MSP827, MSP828, or MSP829. In some embodiments, the TadA variant is MSP828. In some embodiments, the TadA variant is MSP829.

TABLE 5E
TadA Variants

| | TadA Amino Acid Number | | | | | | | |
|---|---|---|---|---|---|---|---|---|
| Variant | 36 | 76 | 82 | 147 | 149 | 154 | 157 | 167 |
| TadA-7.10 | L | I | V | Y | F | Q | N | D |
| MSP605 | | | G | T | | S | | |
| MSP680 | | Y | G | T | | S | | |
| MSP823 | H | | G | T | | S | K | |
| MSP824 | | | G | D | Y | S | | N |
| MSP825 | H | | G | D | Y | S | K | N |
| MSP827 | H | Y | G | T | | S | K | |
| MSP828 | | Y | G | D | Y | S | | N |
| MSP829 | H | Y | G | D | Y | S | K | N |

TABLE 5F
TadA Variants

| | Amino Acid No. | | | | | | | | | |
|---|---|---|---|---|---|---|---|---|---|---|
| | 23 | 36 | 48 | 51 | 76 | 82 | 84 | 106 | 108 | 123 |
| | TadA (wt) | | | | | | | | | |
| Description | W | H | P | R | I | V | L | A | D | H |
| TadA*7.10 | R | L | A | L | I | V | F | V | N | Y |
| TadA*8.8 | | | | | | | | | | H |
| TadA*8.13 | | | | | Y | | | | | H |
| TadA*8.17 | | | | | | S | | | | |
| TadA*8.20 | | | | | Y | S | | | | H |
| TadA*8.8 + V82T | | | | | | T | | | | H |
| TadA*8.8 + V82T + | | | | | | T | | | | H |
| Y147T + Q154S | | | | | | | | | | |
| TadA*8.17 + V82T | | | | | | T | | | | |
| TadA*8.17 + V82T + | | | | | | T | | | | |
| Y147T + Q154S | | | | | | | | | | |
| TadA*8.20 + V82T | | | | | Y | T | | | | H |
| TadA*8.20 + V82T + | | | | | Y | T | | | | H |
| Y147T + Q154S | | | | | | | | | | |

| | Amino Acid No. | | | | | | | |
|---|---|---|---|---|---|---|---|---|
| | 146 | 147 | 152 | 154 | 155 | 156 | 157 | 166 |
| | TadA (wt) | | | | | | | |
| Official Name | S | D | R | Q | E | I | K | T |
| TadA*7.10 | C | Y | P | Q | V | F | N | T |
| TadA*8.8 | | R | | R | | | | |
| TadA*8.13 | | R | | R | | | | |
| TadA*8.17 | | | | R | | | | |
| TadA*8.20 | | R | | R | | | | |
| TadA*8.8 + V82T | | R | | R | | | | |
| TadA*8.8 + V82T + | | T | | S | | | | |
| Y147T + Q154S | | | | | | | | |
| TadA*8.17 + V82T | | | | R | | | | |
| TadA*8.17 + V82T + | | T | | S | | | | |
| Y147T + Q154S | | | | | | | | |
| TadA*8.20 + V82T | | R | | R | | | | |
| TadA*8.20 + V82T + | | T | | S | | | | |
| Y147T + Q154S | | | | | | | | |

In particular embodiments, the fusion proteins or complexes comprise a single (e.g., provided as a monomer) TadA* (e.g., TadA*8 or TadA*9). Throughout the present disclosure, an adenosine deaminase base editor that comprises a single TadA* domain is indicates using the terminology ABEm or ABE #m, where “#” is an identifying number (e.g., ABE8.20m), where “m” indicates “monomer.” In some embodiments, the TadA* is linked to a Cas9 nickase. In some embodiments, the fusion proteins or complexes of the disclosure comprise as a heterodimer of a wild-type TadA (TadA(wt)) linked to a TadA*. Throughout the present disclosure, an adenosine deaminase base editor that comprises a single TadA* domain and a TadA(wt) domain is indicates using the terminology ABEd or ABE #d, where “#” is an identifying number (e.g., ABE8.20d), where “d” indicates “dimer.” In other embodiments, the fusion proteins or complexes of the disclosure comprise as a heterodimer of a TadA*7.10 linked to a TadA*. In some embodiments, the base editor is ABE8 comprising a TadA* variant monomer. In some embodiments, the base editor is ABE comprising a heterodimer of a TadA* and a TadA(wt). In some embodiments, the base editor is ABE comprising a heterodimer of a TadA* and TadA*7.10. In some embodiments, the base editor is ABE comprising a heterodimer of a TadA*. In some embodiments, the TadA* is selected from Tables 5A-5E.

In some embodiments, the adenosine deaminase is expressed as a monomer. In other embodiments, the adenosine deaminase is expressed as a heterodimer. In some embodiments, the deaminase or other polypeptide sequence lacks a methionine, for example when included as a component of a fusion protein. This can alter the numbering of positions. However, the skilled person will understand that such corresponding mutations refer to the same mutation.

Any of the mutations provided herein and any additional mutations (e.g., based on the ecTadA amino acid sequence) can be introduced into any other adenosine deaminases. Any of the mutations provided herein can be made individually or in any combination in a TadA reference sequence or another adenosine deaminase (e.g., ecTadA).

Details of A to G nucleobase editing proteins are described in International PCT Application No. PCT/US2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

### C to T Editing

In some embodiments, a base editor disclosed herein comprises a fusion protein or complex comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, the base editor can comprise a uracil stabilizing protein as described herein. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids.

In some embodiments, a cytidine deaminase of a base editor comprises all or a portion (e.g., a functional portion) of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase.

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Some aspects of the present disclosure are based on the recognition that modulating the deaminase domain catalytic activity of any of the fusion proteins or complexes described herein, for example by making point mutations in the deaminase domain, affect the processivity of the fusion proteins (e.g., base editors) or complexes. For example, mutations that reduce, but do not eliminate, the catalytic activity of a deaminase domain within a base editing fusion protein or complexes can make it less likely that the deaminase domain will catalyze the deamination of a residue adjacent to a target residue, thereby narrowing the deamination window. The ability to narrow the deamination window can prevent unwanted deamination of residues adjacent to specific target residues, which can reduce or prevent off-target effects.

In some embodiments, an APOBEC deaminase incorporated into a base editor can comprise one or more mutations selected from the group consisting of R33A, K34A, E63A, H102P, D104N, H121R, H122R, H122L, D124N; R126A, R126E, R118A, W90A, W90Y, and R132E of rAPOBEC1; D316R, D317R, R320A, R320E, R313A, W285A, W285Y, and R326E of hAPOBEC3G; and any alternative mutation at the corresponding position, or one or more corresponding mutations in another APOBEC deaminase. In some embodiments, an APOBEC deaminase incorporated into a base editor can comprise one or more combinations of mutations selected from K34A, H122L, and D124N (AALN); H102P and D104N (evoFERNY derived from FERNY); W90Y and R126E (YE1); W90Y and R132E (YE2); R126E and R132E (EE); W90Y, R126E, and R132E (YEE), or rAPOBEC1; and any alternative mutation at the corresponding positions, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion (e.g., a functional portion) of an APOBEC1 deaminase.

In some embodiments, the fusion proteins or complexes of the disclosure comprise one or more cytidine deaminase domains. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). Some embodiments provide a polynucleotide molecule encoding the cytidine deaminase nucleobase editor polypeptide of any previous aspect or as delineated herein. In some embodiments, the polynucleotide is codon optimized.

In embodiments, a fusion protein of the disclosure comprises two or more nucleic acid editing domains.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference. Further non-limiting examples of C to T nucleobase editing proteins are described in PCT Applications No. PCT/US2020/062428 and PCT/US2019/033848, the entire contents of which are hereby incorporated by reference.

### Cytidine Adenosine Base Editors (CABEs)

In some embodiments, a base editor described herein comprises an adenosine deaminase variant that has increased cytidine deaminase activity. Such base editors may be referred to as “cytidine adenosine base editors (CABEs)” or “cytosine base editors derived from TadA* (CBE-Ts),” and their corresponding deaminase domains may be referred to as “TadA* acting on DNA cytosine (T_{AD}C)” domains or TadA-derived cytidine deaminases (TadA-CD).

Base editors containing adenosine deaminase variants having both cytidine deamianse and adenosine deaminase activity (i.e., TadA-Dual deaminases) may be referred to as TadA-based dual editors (TadDE). In some instances, an adenosine deaminase variant has both adenine and cytosine deaminase activity (i.e., is a dual deaminase). In some embodiments, the adenosine deaminase variants deaminate adenine and cytosine in DNA. In some embodiments, the adenosine deaminase variants deaminate adenine and cytosine in single-stranded DNA. In some embodiments, the adenosine deaminase variants deaminate adenine and cytosine in RNA. In some embodiments, the adenosine deaminase variant predominantly deaminates cytosine in DNA and/or RNA (e.g., greater than 30%, 40%, 50%, 60%, 70%, 80%, 90%, 95%, or 99% of all deaminations catalyzed by the adenosine deaminase variant, or the number of cytosine deaminations catalyzed by the variant is about or at least about 2-fold, 3-fold, 4-fold, 5-fold, 6-fold, 7-fold, 8-fold, 9-fold, 10-fold, 25-fold, 50-fold, 75-fold, 100-fold, 500-fold, or 1,000-fold greater than the number adenine deaminations catalyzed by the variant). In some embodiments, the adenosine deaminase variant has approximately equal cytosine and adenosine deaminase activity (e.g., the two activities are within about 10% or 20% of each other). In some embodiments, the adenosine deaminase variant has predominantly cytosine deaminase activity, and little, if any, adenosine deaminase activity. In some embodiments, the adenosine deaminase variant has cytosine deaminase activity, and no significant or no detectable adenosine deaminase activity. In some embodiments, the target polynucleotide is present in a cell in vitro or in vivo. In some embodiments, the cell is a bacteria, yeast, fungi, insect, plant, or mammalian cell. Examples of adenosine deaminase variants having increased cytidine deaminase activity include those described in International Patent Application Publications No. WO 2024/040083 and WO 2022/204574, the disclosures of which are hereby incorporated by reference in their entireties for all purposes.

In some embodiments, the CABE comprises a bacterial TadA deaminase variant (e.g., ecTadA). In some embodiments, the CABE comprises a truncated TadA deaminase variant. In some embodiments, the CABE comprises a fragment of a TadA deaminase variant. In some embodiments, the CABE comprises a TadA*8.20 variant.

In some embodiments, an adenosine deaminase variant of the disclosure is a TadA adenosine deaminase comprising one or more alterations that increase cytosine deaminase activity (e.g., at least about 10-fold, 20-fold, 30-fold, 40-fold, 50-fold, 60-fold, 70-fold or more increase) while maintaining adenosine deaminase activity (e.g., at least about 30%, 40%, 50% or more of the activity of a reference adenosine deaminase (e.g., TadA*8.20 or TadA*8.19)). In some instances, the adenosine deaminase variant comprises one or more alterations that increase cytosine deaminase activity (e.g., at least about 10-fold, 20-fold, 30-fold, 40-fold, 50-fold, 60-fold, 70-fold or more increase) relative to the activity of a reference adenosine deaminase and comprise undetectable adenosine deaminase activity or adenosine deaminase activity that is less than 30%, 20%, 10%, or 5% of that of a reference adenosine deaminase. In some embodiments, the reference adenosine deaminase is TadA*8.20 or TadA*8.19.

In some embodiments, the adenosine deaminase variant is an adenosine deaminase comprising two or more alterations at an amino acid position selected from the group consisting of 2, 4, 6, 8, 13, 17, 23, 27, 29, 30, 47, 48, 49, 67, 76, 77, 82, 84, 96, 100, 107, 112, 114, 115, 118, 119, 122, 127, 142, 143, 147, 149, 158, 159, 162 165, 166, and 167, of an amino acid sequence having at least about 70%, 75%, 80%, 85%, 90%, 95%, 98%, 99% or greater identity to SEQ ID NO: 1, or a corresponding alteration in another deaminase. I

In some embodiments, the adenosine deaminase variant is an adenosine deaminase comprising one or more alterations selected from the group consisting of S2H, V4K, V4S, V4T, V4Y, F6G, F6H, F6Y, H8Q, R13G, T17A, T17W, R23Q, E27C, E27G, E27H, E27K, E27Q, E27S, E27G, P29A, P29G, P29K, V30F, V30I, R47G, R47S, A48G, 149K, 149M, 149N, 149Q, I49T, G67W, I76H, I76R, I76W, Y76H, Y76R, Y76W, F84A, F84M, H96N, G100A, G100K, T111H, G112H, A114C, G115M, M118L, H122G, H122R, H122T, N127I, N127K, N127P, A142E, R147H, A158V, Q159S, A162C, A162N, A162Q, and S165P of an amino acid sequence having at least about 70%, 75%, 80%, 85%, 90%, 95%, 98%, 99% or greater identity to SEQ ID NO: 1, or a corresponding alteration in another deaminase.

In some embodiments, the adenosine deaminase variant is an adenosine deaminase comprising an amino acid alteration or combination of amino acid alterations selected from those listed in any of Tables 6A-6F.

The residue identity of exemplary adenosine deaminase variants that are capable of deaminating adenine and/or cytidine in a target polynucleotide (e.g., DNA) is provided in Tables 6A-6F below. Further examples of adenosine deaminase variants include the following variants of 1.17 (see Table 6A): 1.17+E27H; 1.17+E27K; 1.17+E27S; 1.17+E27S+I49K; 1.17+E27G; 1.17+I49N; 1.17+E27G+I49N; and 1.17+E27Q. In some embodiments, any of the amino acid alterations provided herein are substituted with a conservative amino acid. Additional mutations known in the art can be further added to any of the adenosine deaminase variants provided herein.

In some embodiments, the base editor systems comprising a CABE provided herein have at least about a 30%, 40%, 50%, 60%, 70% or more C to T editing activity in a target polynucleotide (e.g., DNA). In some embodiments, a base editor system comprising a CABE as provided herein has an increased C to T base editing activity (e.g., increased at least about 30-fold, 40-fold, 50-fold, 60-fold, 70-fold or more) relative to a reference base editor system comprising a reference adenosine deaminase (e.g., TadA*8.20 or TadA*8.19).

TABLE 6A
Adenosine Deaminase Variants. Mutations are indicated with reference to TadA*8.20.

| | location in structure | | | | | | | | | | |
|---|---|---|---|---|---|---|---|---|---|---|---|
| | N/A | S h1 | S h1 | S h1 | NAS | NAS | NAS | NAS | | S | |
| | Amino Acid No. (*START Met is AA#1) | | | | | | | | | | |
| | 2 | 8 | 13 | 17 | 27 | 47 | 48 | 49 | 67 | 76 | 77 |
| TadA*8.20 | S | H | R | T | E | R | A | I | G | Y | D |
| TadA*8.19 | | | | | | | | | | I | |
| 1.1 | | | | | H | | | | | I | |
| 1.2 | | | | | H | | | K | | I | |
| 1.3 | | | | | S | | | K | | I | |
| 1.4 | | | | | S | | | K | | I | |
| 1.5 | | | | | K | | | | | | |
| 1.6 | | | | | K | | | | | | |
| 1.7 | | | | | H | | | | | I | |
| 1.8 | | | | | S | | | K | W | | |
| 1.9 | | | | | | | | T | W | | |
| 1.10 | | | | | C | | | | | I | |
| 1.11 | | | G | | Q | | | | | | |
| 1.12 | | | | A | H | | | M | | I | |
| 1.13 | | | | | | | | Q | | T | |
| 1.14 | H | | | | | | | K | | I | |
| 1.15 | | | | | | S | | | | | |
| 1.16 | | Q | | | | | | Q | | I | |
| 1.17 | | | | A | | | G | | | | |
| 1.18 | | | | | G | | | | | | |
| 1.19 | | | | | G | | | N | | | |
| 1.20 | | | | | G | | | | | | G |

| | location in structure | | | | | | | | | | | |
|---|---|---|---|---|---|---|---|---|---|---|---|---|
| | I | NAS | | NAS | S | | S | S | S | | S | |
| | Amino Acid No. (*START Met is AA#1) | | | | | | | | | | | |
| | 82 | 84 | 96 | 107 | 112 | 115 | 118 | 119 | 127 | 142 | 162 | 165 |
| TadA*8.20 | S | F | H | R | G | G | M | D | N | A | A | S |
| TadA*8.19 | | | | | | | | | | | | |
| 1.1 | | M | | | | | | | | | | |
| 1.2 | | | | | | | | | | | | |
| 1.3 | | | | | | | | | | | | |
| 1.4 | | | | | | | | | | | N | |
| 1.5 | | | | | | | | | | | | |
| 1.6 | | | | | | | | N | | | | |
| 1.7 | | | | | | | | | | | | |
| 1.8 | | | | | | | | | | | | |
| 1.9 | | | N | | | | | | | | | |
| 1.10 | | | | | | | | N | | | | |
| 1.11 | | | | | | | | | K | | | |
| 1.12 | | | | | | | L | | | | | |
| 1.13 | | | | | | M | | | | | | |
| 1.14 | | | | | H | | | | | | | |
| 1.15 | | | | C | | | | | | | | |
| 1.16 | | | | | | | | | | | | |
| 1.17 | T | | | | | | | | | E | | |
| 1.18 | | | | | | | | | | | | |
| 1.19 | | | | | | | | | | | | |
| 1.20 | | | | | | | | | | | | P |

“I” indicates “Internal,” “S” indicates “Surface,” and “NAS” indicates “Near Active Site.”

TABLE 6B
Adenosine deaminase variants. Mutations are
indicated with reference to TadA*8.20.

| | Position No. | | | | | | | | | |
|---|---|---|---|---|---|---|---|---|---|---|
| | 27 | 29 | 30 | 49 | 82 | 84 | 107 | 112 | 115 | 142 |
| | TadA*8.20 | | | | | | | | | |
| | E | P | V | I | S | F | R | G | G | A |
| | Alterations Evaluated | | | | | | | | | |
| | G/S/H | G/A/K | I/L/F | K | T | L/A | C | H | M | E |
| S1.1 | S | | | K | T | | | | | |
| S1.2 | S | | | K | T | | C | | | |
| S1.3 | S | | | K | T | | | H | | |
| S1.4 | S | | | K | T | | | | M | |
| S1.5 | S | | | K | T | | | | | E |
| S1.6 | S | | | K | T | | C | H | | |
| S1.7 | S | | | K | T | | C | | M | |
| S1.8 | S | | | K | T | | C | | | E |
| S1.9 | S | | | K | T | | | H | | E |
| S1.10 | S | | | K | T | | | | M | E |
| S1.11 | S | | | K | T | | C | H | M | E |
| S1.12 | S | | I | K | T | | | | | |
| S1.13 | S | | I | K | T | | C | | | |
| S1.14 | S | | I | K | T | | | H | | |
| S1.15 | S | | I | K | T | | | | M | |
| S1.16 | S | | I | K | T | | | | | E |
| S1.17 | S | | I | K | T | | C | H | | |
| S1.18 | S | | I | K | T | | C | | M | |
| S1.19 | S | | I | K | T | | C | | | E |
| S1.20 | S | | I | K | T | | | H | | E |
| S1.21 | S | | I | K | T | | | | M | E |
| S1.22 | S | | I | K | T | | C | H | M | E |
| S1.23 | S | | L | K | T | | | | | |
| S1.24 | S | | T | K | T | | C | | | |
| S1.25 | S | | L | K | T | | | H | | |
| S1.26 | S | | T | K | T | | | | M | |
| S1.27 | S | | L | K | T | | | | | E |
| S1.28 | S | | T | K | T | | C | H | | |
| S1.29 | S | | I | K | T | | C | | M | |
| $1.30 | S | | L | K | T | | C | | | E |
| S1.31 | S | | L | K | T | | | H | | E |
| S1.32 | S | | H | K | T | | | | M | E |
| S1.33 | S | | T | K | T | | C | H | M | E |
| S1.34 | S | | F | K | T | A | | | | |
| S1.35 | S | | F | K | T | A | C | | | |
| S1.36 | S | | F | K | T | A | | H | | |
| S1.37 | S | | F | K | T | A | | | M | |
| S1.38 | S | | F | K | T | A | | | | E |
| S1.39 | S | | F | K | T | A | C | H | | |
| S1.40 | S | | F | K | T | A | C | | M | |
| S1.41 | S | | F | K | T | A | C | | | E |
| S1.42 | S | | F | K | T | A | | H | | E |
| S1.43 | S | | F | K | T | A | | | M | E |
| S1.44 | S | | F | K | T | A | C | H | M | E |
| S1.45 | S | | | K | T | L | | | | |
| S1.46 | S | | | K | T | L | C | | | |
| S1.47 | S | | | K | T | L | | H | | |
| S1.48 | S | | | K | T | L | | | M | |
| S1.49 | S | | | K | T | L | | | | E |
| S1.50 | S | | | K | T | L | C | H | | |
| S1.51 | S | | | K | T | L | C | | M | |
| S1.52 | S | | | K | T | L | C | | | E |
| S1.53 | S | | | K | T | L | | H | | E |
| S1.54 | S | | | K | T | L | | | M | E |
| S1.55 | S | | | K | T | L | C | H | M | E |
| S1.56 | S | | I | K | T | L | | | | |
| S1.57 | S | | | K | T | L | C | | | |
| S1.58 | S | | T | K | T | 1 | | H | | |
| $1.59 | S | | I | K | T | L | | | M | |
| S1.60 | S | | I | K | T | L | | | | E |
| S1.61 | S | | I | K | T | L | C | H | | |
| S1.62 | S | | I | K | T | L | C | | M | |
| S1.63 | S | | I | K | T | L | C | | | E |
| S1.64 | S | | H | K | T | L | | H | | E |
| S1.65 | S | | H | K | T | L | | | M | E |
| S1.66 | S | | I | K | T | L | C | H | M | E |
| S1.67 | S | G | | K | T | | | | | |
| S1.68 | S | G | | K | T | | C | | | |
| S1.69 | S | G | | K | T | | | H | | |
| S1.70 | S | G | | K | T | | | | M | |
| S1.71 | S | G | | K | T | | | | | E |
| S1.72 | S | G | | K | T | | C | H | | |
| S1.73 | S | G | | K | T | | C | | M | |
| S1.74 | S | G | | K | T | | C | | | E |
| S1.75 | S | G | | K | T | | | H | | E |
| S1.76 | S | G | | K | T | | | | M | E |
| S1.77 | S | G | | K | T | | C | H | M | E |
| S1.78 | | G | | K | T | | | | | |
| S1.79 | | G | | K | T | | ? | | | |
| S1.80 | | G | | K | T | | | H | | |
| S1.81 | | G | | K | T | | | | M | |
| S1.82 | | G | | K | T | | | | | E |
| S1.83 | | G | | K | T | | C | H | | |
| S1.84 | | G | | K | T | | C | | M | |
| S1.85 | | G | | K | T | | C | | | E |
| S1.86 | | G | | K | T | | | H | | E |
| S1.87 | | G | | K | T | | | | M | E |
| S1.88 | | G | | K | T | | C | H | M | E |
| S1.89 | | K | | K | T | | | | | |
| $1.90 | | K | | K | T | | C | | | |
| S1.91 | | K | | K | T | | | H | | |
| S1.92 | | K | | K | T | | | | M | |
| S1.93 | | K | | K | T | | | | | E |
| S1.94 | | K | | K | T | | C | H | | |
| S1.95 | | K | | K | T | | C | | M | |
| S1.96 | | K | | K | T | | C | | | E |
| S1.97 | | K | | K | T | | | H | | E |
| $1.98 | | K | | K | T | | | | M | E |
| $1.99 | | K | | K | T | | C | H | M | E |
| S1.100 | | K | I | K | T | | | | | |
| S1.101 | | K | I | K | T | | C | | | |
| S1.102 | | K | I | K | T | | | H | | |
| S1.103 | | K | I | K | T | | | | M | |
| S1.104 | | K | I | K | T | | | | | E |
| S1.105 | | K | I | K | T | | C | H | | |
| S1.106 | | K | I | K | T | | C | | M | |
| S1.107 | | K | ? | K | T | | C | | | E |
| S1.108 | | K | T | K | T | | | H | | E |
| S1.109 | | K | I | K | T | | | | M | E |
| S1.110 | | K | ? | K | T | | C | H | M | E |
| S1.111 | | K | | K | T | L | | | | |
| S1.112 | | K | | K | T | L | C | | | |
| S1.113 | | K | | K | T | L | | H | | |
| S1.114 | | K | | K | T | L | | | M | |
| S1.115 | | K | | K | T | L | | | | E |
| S1.116 | | K | | K | T | L | C | H | | |
| S1.117 | | K | | K | T | L | C | | M | |
| S1.118 | | K | | K | T | L | C | | | E |
| S1.119 | | K | | K | T | L | | H | | E |
| S1.120 | | K | | K | T | L | | | M | E |
| S1.121 | | K | | K | T | L | C | H | M | E |
| S1.122 | | K | I | K | T | L | | | | |
| S1.123 | | K | I | K | T | L | C | | | |
| S1.124 | | K | T | K | T | L | | H | | |
| S1.125 | | K | I | K | T | L | | | M | |
| S1.126 | | K | I | K | T | L | | | | E |
| S1.127 | | K | I | K | T | L | C | H | | |
| S1.128 | | K | ? | K | T | L | C | | M | |
| S1.129 | | K | I | K | T | L | C | | | E |
| S1.130 | | K | I | K | T | L | | H | | E |
| S1.131 | | K | I | K | T | L | | | M | E |
| S1.132 | | K | I | K | T | L | C | H | M | E |
| S1.133 | G | | | K | T | | | | | |
| S1.134 | G | | | K | T | | C | | | |
| S1.135 | G | | | K | T | | | H | | |
| S1.136 | G | | | K | T | | | | M | |
| S1.137 | G | | | K | T | | | | | E |
| S1.138 | G | | | K | T | | C | H | | |
| S1.139 | G | | | K | T | | C | | M | |
| S1.140 | G | | | K | T | | C | | | F |
| S1.141 | G | | | K | T | | | H | | E |
| S1.142 | G | | | K | T | | | | M | E |
| S1.143 | G | | | K | T | | C | H | M | E |
| S1.144 | H | | | K | T | | | | | |
| S1.145 | H | | | K | T | | C | | | |
| S1.146 | H | | | K | T | | | H | | |
| S1.147 | H | | | K | T | | | | M | |
| S1.148 | H | | | K | T | | | | | E |
| S1.149 | H | | | K | T | | C | H | | |
| S1.150 | H | | | K | T | | C | | M | |
| S1.151 | H | | | K | T | | C | | | E |
| S1.152 | H | | | K | T | | | H | | |
| S1.153 | H | | | K | T | | | | M | E |
| S1.154 | H | | | K | T | | C | H | M | E |
| S1.155 | S | | | | T | | | | | |
| S1.156 | S | | | | | | C | | | |
| S1.157 | S | | | | | | | H | | |
| S1.158 | S | | | | | | | | M | |
| S1.159 | S | | | | | | | | | E |
| S1.160 | S | | | | | | C | H | | |
| S1.161 | S | | | | | | | | M | |
| S1.162 | S | | | | | | C | | | E |
| S1.163 | S | | | | | | | H | | E |
| S1.164 | S | | | | | | | | M | E |
| S1.165 | S | | | | | | C | H | M | E |
| S1.166 | | A | | | | | | | | |
| S1.167 | | A | | | | | C | | | |
| S1.168 | | A | | | | | | H | | |
| S1.169 | | A | | | | | | | M | |
| S1.170 | | A | | | | | | | | E |
| S1.171 | | A | | | | | C | H | | |
| S1.172 | | A | | | | | | | M | |
| S1.173 | | A | | | | | C | | | E |
| S1.174 | | A | | | | | | H | | E |
| S1.175 | | A | | | | | | | M | E |
| S1.176 | | A | | | H | | C | H | M | E |
| S1.177 | S | | I | | | | | | | |
| S1.178 | S | | I | | | | C | | | |
| S1.179 | S | | I | | | | | H | | |
| S1.180 | S | | ? | | | | | | M | |
| S1.181 | S | | I | | | | | | | E |
| S1.182 | S | | I | | T | | C | H | | |
| S1.183 | S | | I | | | | C | | M | |
| S1.184 | S | | I | | | | C | | | E |
| S1.185 | S | | T | | | | | H | | E |
| S1.186 | S | | I | | | | | | M | E |
| S1.187 | S | | I | | | | C | H | M | E |
| S1.188 | | A | I | | | L | | | | |
| S1.189 | | A | I | | | L | C | | | |
| S1.190 | | A | I | | | L | | H | | |
| S1.191 | | A | I | | | L | | | M | |
| S1.192 | | A | I | | T | L | | | | E |
| S1.193 | | A | I | | T | L | C | H | | |
| S1.194 | | A | I | | | L | C | | M | |
| S1.195 | | A | I | | T | L | C | | | E |
| S1.196 | | A | I | | T | L | | H | | E |
| S1.197 | | A | I | | T | L | | | M | E |
| S1.198 | | A | I | | T | L | C | H | M | E |
| S1.199 | S | A | L | K | T | L | C | H | M | E |

TABLE 6C
Adenosine deaminase variants. Mutations are indicated with reference to variant 1.2 (Table 6A) .

| | | Residue identity (START Met is amino | | | | | | | | |
|---|---|---|---|---|---|---|---|---|---|---|
| Variant | Alternative Variant | acid #1) | | | | | | | | |
| Name | Names | 4 | 6 | 17 | 23 | 79 | 77 | 100 | 111 | 114 |
| Reference | 1.2 (see Table 6A) | V | F | T | R | I | D | G | T | A |
| TadAC2.1 | pDKL-135; 2.1 | K | | | | | | | | C |
| TadAC2.2 | pDKL-136; 2.2 | K | | | | | G | | | |
| TadAC2.3 | pDKL-137; 2.3 | | Y | | | | | A | | |
| TadAC2.4 | pDKL-138; 2.4 | T | | | | R | | | | |
| TadAC2.5 | pDKL-139; 2.5 | | Y | | | W | | | | |
| TadAC2.6 | pDKL-140; 2.6 | | Y | | | | | | | |
| TadAC2.7 | pDKL-141; 2.7 | | Y | | | | | | | C |
| TadAC2.8 | pDKL-142; 2.8 | | Y | | | | | | | |
| TadAC2.9 | pDKL-143; 2.9 | K | | | | W | | | | |
| TadAC2.10 | pDKL-144; 2.10 | | G | | | R | | K | | |
| TadAC2.11 | pDKL-145; 2.11 | | H | | | | | | | |
| TadAC2.12 | pDKL-146; 2.12 | | | | | | | | | C |
| TadAC2.13 | pDKL-147; 2.13 | | Y | | | H | | | | |
| TadAC2.14 | pDKL-148; 2.14 | | | | | | | | | |
| TadAC2.15 | pDKL-149; 2.15 | | | | Q | R | | | | |
| TadAC2.16 | pDKL-150; 2.16 | | | | | H | | | | |
| TadAC2.17 | pDKL-151; 2.17 | | Y | | | | | | H | |
| TadAC2.18 | pDKL-152; 2.18 | | | | | W | | | | |
| TadAC2.19 | pDKL-153; 2.19 | | | | | | | | H | |
| TadAC2.20 | pDKL-154; 2.20 | | | | | | | | | |
| TadAC2.21 | pDKL-155; 2.21 | | Y | | | R | | | | |
| TadAC2.22 | pDKL-156; 2.22 | | | M | | H | | | | |
| TadAC2.23 | pDKL-157; 2.23 | S | | | | Y | | | | |
| TadAC2.24 | pDKL-158; 2.24 | | | | | | | | | |

| | | Residue identity (START Met is | | | | | | | | |
|---|---|---|---|---|---|---|---|---|---|---|
| | Alternative Variant | amino acid #1) | | | | | | | | |
| Variant Name | Names | 119 | 122 | 127 | 143 | 147 | 158 | 159 | 162 | 166 |
| Reference | 1.2 (see Table 6A) | D | H | N | A | R | A | Q | A | T |
| TadAC2.1 | pDKL-135; 2.1 | | | | | | | | | |
| TadAC2.2 | pDKL-136; 2.2 | | | | | | | | | |
| TadAC2.3 | pDKL-137; 2.3 | | R | | | | | | | |
| TadAC2.4 | pDKL-138; 2.4 | | G | | | | | | | |
| TadAC2.5 | pDKL-139; 2.5 | | | | | | | | | |
| TadAC2.6 | pDKL-140; 2.6 | N | | | | | | | | |
| TadAC2.7 | pDKL-141; 2.7 | | | | | | | | | |
| TadAC2.8 | pDKL-142; 2.8 | | | | | | | | | |
| TadAC2.9 | pDKL-143; 2.9 | | T | | | | | | | |
| TadAC2.10 | pDKL-144; 2.10 | | | | | | | | | |
| TadAC2.11 | pDKL-145; 2.11 | | N | | | | | | | |
| TadAC2.12 | pDKL-146; 2.12 | | | | | | | | | |
| TadAC2.13 | pDKL-147; 2.13 | | R | | | | | | | I |
| TadAC2.14 | pDKL-148; 2.14 | | | P | | | | | | |
| TadAC2.15 | pDKL-149; 2.15 | | | | | | | | | |
| TadAC2.16 | pDKL-150; 2.16 | | R | | | | V | | | |
| TadAC2.17 | pDKL-151; 2.17 | | | | | | | | | |
| TadAC2.18 | pDKL-152; 2.18 | | | | | | | | | |
| TadAC2.19 | pDKL-153; 2.19 | | G | | | | | | C | |
| TadAC2.20 | pDKL-154; 2.20 | | | | E | | | | | |
| TadAC2.21 | pDKL-155; 2.21 | | | | | | | | | |
| TadAC2.22 | pDKL-156; 2.22 | | G | | | | V | | | |
| TadAC2.23 | pDKL-157; 2.23 | | | | E | | | S | | |
| TadAC2.24 | pDKL-158; 2.24 | | | I | | | | | Q | |

TABLE 6D
Adenosine deaminase variants. Mutations are indicated with reference to TadA*8.20.

| | AA Positions | | | | | | | | | | | | | | |
|---|---|---|---|---|---|---|---|---|---|---|---|---|---|---|---|
| | 6 | 27 | 49 | 76 | 77 | 82 | 107 | 112 | 114 | 115 | 119 | 122 | 127 | 142 | 143 |
| | TadA*8.20 | | | | | | | | | | | | | | |
| | F | E | I | Y | D | S | R | G | A | G | D | H | N | A | A |
| S1.154 | F | H | K | Y | D | T | C | H | | M | | | | E | |
| Alterations | Y | | | W | G | | | | C | | N | G | P | | E |
| from Table | | | | | | | | | | | | | | | |
| 6C | | | | | | | | | | | | | | | |
| S2.1 | Y | H | K | W | | T | C | H | | M | | | | E | |
| S2.2 | Y | H | K | | G | T | C | H | | M | | | | E | |
| S2.3 | Y | H | K | | | T | C | H | C | M | | | | E | |
| S2.4 | Y | H | K | | | T | C | H | | M | N | | | E | |
| S2.5 | Y | H | K | | | T | C | H | | M | | G | | E | |
| S2.6 | Y | H | K | | | T | C | H | | M | | | P | E | |
| S2.7 | Y | H | K | | | T | C | H | | M | | | | E | E |
| S2.8 | Y | H | K | | | T | C | H | | M | | | | A | E |
| S2.9 | Y | H | K | W | G | T | C | H | | M | | | | E | |
| S2.10 | Y | H | K | W | | T | C | H | C | M | | | | E | |
| S2.11 | Y | H | K | W | | T | C | H | | M | N | | | E | |
| S2.12 | Y | H | K | W | | T | C | H | | M | | G | | E | |
| S2.13 | Y | H | K | W | | T | C | H | | M | | | P | E | |
| S2.14 | Y | H | K | W | | T | C | H | | M | | | | E | E |
| S2.15 | Y | H | K | W | | T | C | H | | M | | | | A | E |
| S2.16 | Y | H | K | | G | T | C | H | C | M | | | | E | |
| S2.17 | Y | H | K | | | T | C | H | | M | N | | | E | |
| S2.18 | Y | H | K | | G | T | C | H | | M | | G | | E | |
| S2.19 | Y | H | K | | G | T | C | H | | M | | | P | E | |
| S2.20 | Y | H | K | | G | T | C | H | | M | | | | E | E |
| S2.21 | Y | H | K | | G | T | C | H | | M | | | | A | E |
| S2.22 | Y | H | K | | | T | C | H | C | M | N | | | E | |
| S2.23 | Y | H | K | | | T | C | H | C | M | | G | | E | |
| S2.24 | Y | H | K | | | T | C | H | C | M | | | P | E | |
| S2.25 | Y | H | K | | | T | C | H | | M | N | G | | E | |
| S2.26 | Y | H | K | | | T | C | H | | M | N | | P | E | |
| S2.27 | Y | H | K | | | T | C | H | | M | | G | P | E | |
| S2.28 | Y | H | K | W | G | T | C | H | C | M | | | | E | |
| S2.29 | Y | H | K | W | G | T | C | H | | M | N | | | E | |
| S2.30 | Y | H | K | W | G | T | C | H | | M | | G | | E | |
| S2.31 | Y | H | K | W | G | T | C | H | | M | | | P | E | |
| S2.32 | Y | H | K | W | G | T | C | H | | M | | | | E | E |
| S2.33 | Y | H | K | W | G | T | C | H | | M | | | | A | E |
| S2.34 | Y | H | K | W | | T | C | H | C | M | N | | | E | |
| S2.35 | Y | H | K | W | | T | C | H | C | M | | G | | E | |
| S2.36 | Y | H | K | W | | T | C | H | C | M | | | P | E | |
| S2.37 | Y | H | K | W | | T | C | H | C | M | | | | E | E |
| S2.38 | Y | H | K | W | | T | C | H | C | M | | | | A | E |
| S2.39 | Y | H | K | W | | T | C | H | | M | N | G | | E | |
| S2.40 | Y | H | K | W | | T | C | H | | M | N | | P | E | |
| S2.41 | Y | H | K | W | | T | C | H | | M | | G | P | E | |
| S2.42 | Y | H | K | W | | T | C | H | C | M | N | G | | E | |
| S2.43 | Y | H | K | W | | T | C | H | C | M | N | | P | E | |
| S2.44 | Y | H | K | W | | T | C | H | C | M | | G | P | E | |
| S2.45 | Y | H | K | W | G | T | C | H | C | M | N | | | E | |
| S2.46 | Y | H | K | W | G | T | C | H | C | M | | G | | E | |
| S2.47 | Y | H | K | W | G | T | C | H | C | M | | | P | E | |
| S2.48 | Y | H | K | W | G | T | C | H | C | M | | | | E | E |
| S2.49 | Y | H | K | W | G | T | C | H | C | M | | | | A | E |
| S2.50 | Y | H | K | W | G | T | C | H | C | M | N | G | | E | |
| S2.51 | Y | H | K | W | G | T | C | H | C | M | N | | P | E | |
| S2.52 | Y | H | K | W | G | T | C | H | C | M | | G | P | E | |
| S2.53 | Y | H | K | W | | T | C | H | C | M | N | G | P | E | E |
| S2.54 | Y | H | K | W | | T | C | H | C | M | N | G | P | A | E |
| S2.55 | Y | H | K | W | G | T | C | H | C | M | N | G | P | E | E |
| S2.56 | Y | H | K | W | G | T | C | H | C | M | N | G | P | A | E |

TABLE 6E
Hybrid constructs. Mutations are indicated with reference to TadA*7.10.

| | TadA amino acid subsitutions | | | | | | | | | | | |
|---|---|---|---|---|---|---|---|---|---|---|---|---|
| | 76 | 82 | 109 | 111 | 119 | 122 | 123 | 147 | 149 | 154 | 166 | 167 |
| | TadA*7.10 | | | | | | | | | | | |
| | I | V | A | T | D | H | Y | Y | F | Q | T | D |
| | TadA*8e | | | | | | | | | | | |
| | | | S | R | N | N | | D | Y | | I | N |
| | TadA*8.20 | | | | | | | | | | | |
| | Y | S | | | | | H | R | | R | | |
| | TadA*8.17 | | | | | | | | | | | |
| | | S | | | | | | | | R | | |
| pNMG-B878 | Y | S | | | | | H | D | | R | | |
| pNMG-B879 | Y | S | | | | | H | R | Y | R | | |
| pNMG-B880 | Y | S | | | | | H | R | | R | I | |
| pNMG-B881 | Y | S | | | | | H | R | | R | | N |
| pNMG-B882 | Y | S | | | | | H | D | Y | R | I | N |
| pNMG-B883 | Y | S | | R | N | | H | R | | R | | |
| pNMG-B884 | Y | S | S | R | N | N | H | R | | R | | |
| pNMG-B885 | Y | S | S | | | | H | R | | R | | |
| pNMG-B886 | Y | S | | R | | | H | R | | R | | |
| pNMG-B887 | Y | S | | | N | | H | R | | R | | |
| pNMG-B888 | Y | S | | | | N | H | R | | R | | |
| pNMG-B889 | Y | S | S | R | | | H | R | | R | | |
| pNMG-B890 | Y | S | | | N | N | H | R | | R | | |
| pNMG-B891 | Y | S | S | R | N | N | H | D | Y | R | I | N |

TABLE 6F
Base editor variants. Mutations are indicated with reference to TadA*8.19/8.20.

| | AA positions: | | | | | | | | | | | | |
|---|---|---|---|---|---|---|---|---|---|---|---|---|---|
| | 17 | 27 | 48 | 49 | 76 | 82 | 84 | 118 | 142 | 147 | 149 | 166 | 167 |
| | ABE8.19m/8.20m | | | | | | | | | | | | |
| | T | E | A | I | Y/I | S | F | M | A | Y | F | T | D |
| 1.1 + 8e(B879) | | H | | | I | | M | | | | Y | | |
| 1.2 + 8e(B879) | | H | | K | I | | | | | | Y | | |
| 1.12 + 8e(B879) | A | H | | M | I | | | L | | | Y | | |
| 1.17 + 8e(B879) | A | | G | | | T | | | E | | Y | | |
| 1.18 + 8e(B879) | | G | | | | | | | | | Y | | |
| 1.19 + 8e(B879) | | G | | N | | | | | | | Y | | |
| 1.1 + 8e(B882) | | H | | | I | | M | | | D | Y | I | N |
| 1.2 + 8e(B882) | | H | | K | I | | | | | D | Y | I | N |
| 1.12 + 8e(B882) | A | H | | M | I | | | L | | D | Y | I | N |
| 1.17 + 8e(B882) | A | | G | | | T | | | E | D | Y | I | N |
| 1.18 + 8e(B882) | | G | | | | | | | | D | Y | I | N |
| 1.19 + 8e(B882) | | G | | N | | | | | | D | Y | I | N |

A TadA-derived cytidine deaminase (e.g., TadA-CD), according to certain embodiments, comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 489, wherein residue 27 of SEQ ID NO: 489 is any amino acid expect for E (glutamic acid). TadA-CDs with other sequence homologies are also possible. For example, in certain embodiments, the TadA-derived cytidine deaminase (e.g., TadA-CD) comprises an amino acid sequence that is at least 80% identical, at least 85% identical, at least 90% identical, at least 95% identical, at least 98% identical, at least 99% identical, and at least 99.5% identical to the amino acid sequence of SEQ ID NO: 489, wherein residue 28 of SEQ ID NO: 489 is any amino acid expect for V (valine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 489, wherein residue 96 of SEQ ID NO: 489 is any amino acid expect for H (histidine). In another exemplary embodiment, the TadA-derived cytidine deaminase is at least 80% identical, at least 85% identical, at least 90% identical, at least 95% identical, at least 98% identical, at least 99% identical, and at least 99.5% identical to the amino acid sequence of SEQ ID NO: 489, wherein residue 26 of SEQ ID NO: 489 is any amino acid expect for R (arginine). In various embodiments, the TadA-derived cytidine deaminase comprises an alteration at one or more of positions 26, 27, 28, 48, 73, or 96 compared to SEQ ID NO: 489.

As will be appreciated by those of skill in the art, TadA-derived cytidine deaminases (e.g., TadA-CD) may comprise a plurality of mutations relative to the parent adenosine deaminase (e.g., TadA-8e). In some embodiments, the deaminase of the instant application (e.g., TadA-CD) comprises mutations at residues E27, V28, and H96. In some embodiments, the disclosed deaminase further comprises at least one mutation at a residue selected from R26, M61, Y73, 176, M151, Q154, and A158, in the amino acid sequence of SEQ ID NO: 489, or corresponding mutations in a homologous adenosine deaminase.

In some embodiments, the deaminase comprises at least one mutation selected from E27A, E27K, V28G, V28A, and H96N, and further comprises at least one mutation at a residue selected from R26G, M61I, Y73H, Y73S, Y73C, I76F, M151I, Q154R, Q154H, and A158S, in the amino acid sequence of SEQ ID NO: 489, or a corresponding mutation in a homologous adenosine deaminase. Other mutations are also possible. For example, in certain embodiments, the TadA-CD enzyme comprises mutations selected from E27A, V28G, and H96N, and further comprises at least one mutation selected from R26G, M61I, Y73H, Y73S, Y73C, I76F, M151I, Q154R, Q154H, and A158S, in the amino acid sequence of SEQ ID NO: 489, or corresponding mutations in a homologous adenosine deaminase.

Other exemplary embodiments may include (1) deaminases comprising mutations E27K, V28G, and H96N, and further comprising at least one mutation selected from R26G, M61I, Y73H, Y73S, Y73C, I76F, M151I, Q154R, Q154H, and A158S, in the amino acid sequence of SEQ ID NO: 489 or corresponding mutations in a homologous adenosine deaminase; (2) deaminases comprising mutations E27A, V28A, and H96N, and further comprising at least one mutation selected from R26G, M61I, Y73H, Y73S, Y73C, I76F, M151I, Q154R, Q154H, and A158S, in the amino acid sequence of SEQ ID NO: 489, or corresponding mutations in a homologous adenosine deaminase; (3) deaminases comprising mutations E27K, V28A, and H96N, and further comprising at least one mutation selected from R26G, M61I, Y73H, Y73S, Y73C, I76F, M151I, Q154R, Q154H, and A158S, in the amino acid sequence of SEQ ID NO: 489, or corresponding mutations in a homologous adenosine deaminase.

In some embodiments, the TadA-derived cytidine deaminases (TadA-CD) comprise at least two mutations at residues selected from R26, M61, Y73, 176, M151, Q154, and A158 (relative to a reference adenosine deaminase). In other embodiments, the TadA-CD comprises at least two mutations at residues selected from R26G, M61I, Y73H, I76F, M151I, Q154H, Q154R, and A158S.

In some embodiments, the addition of a V106W mutation improves the selectivity by suppressing A deamination to a greater extent than C deamination.

In some embodiments, a TadA-based dual editor comprises an adenosine deaminase variant comprising one, two, three, four, or five mutations selected from R26G, V28A, A48R, Y73S, and H96N (e.g., SEQ ID NO: 495).

As such, in some embodiments, provided herein are deaminases that comprise mutations at residues R26, V28, A48, and Y73 in the amino acid sequence of SEQ ID NO: 489, or corresponding mutations in a homologous adenosine deaminase. Further provided herein are deaminases that comprise mutations at residues R26, E27, V28, A48, and Y73 (e.g., further comprise a mutation at E27) in the amino acid sequence of SEQ ID NO: 489. In particular embodiments, these deaminases comprise the mutations R26G, V28A, A48R, Y73S, and H96N. In some embodiments, these deaminases comprise the mutations R26G, V28G, A48R, and Y73C.

TadA-CD variants may comprise at least one mutation selected from R26G, E27A, V28G, I76F, H96N, and M151I (e.g, TadA-CDa, SEQ ID NO: 490); R26G, E27A, V28G, I76F, H96N, and A158S (e.g, TadA-CDb, SEQ ID NO: 491); R26G, E27A, V28G, I76F, H96N, Q154R, and A158S (e.g, TadA-CDc, SEQ ID NO: 492); E27A, V28G, Y73H, H96N, Q154H, and A158S (e.g., TadA-CDd, SEQ ID NO: 493); R26G, V28A, A48R, Y73S, and H96N (e.g., TadA-CDe, SEQ ID NO: 494); V28A, A48R, and Y73S (e.g, TadA-CDf, SEQ ID NO: 495), and R26G, V28G, A48R, and Y73C (e.g, TadA-CDg, SEQ ID NO: 496).

In some preferred embodiments, the deaminase comprises the mutations R26G, E27A, V28G, I76F, H96N, and A158S (e.g., TadA-CDa, SEQ ID NO: 490), R26G, E27A, V28G, I76F, H96N, Q154R, and A158S (e.g., TadA-CDb, SEQ ID NO: 491), R26G, E27A, V28G, I76F, H96N, and M151I (e.g., TadA-CDc, SEQ ID NO: 492), E27K, V28A, M61I, and H96N (e.g., TadA-CDd, SEQ ID NO: 493), E27A, V28G, Y73H, H96N, Q154H, and A158S (e.g., TadA-CDe, SEQ ID NO: 494), R26G, V28A, A48R, Y73S, and H96N (e.g., TadA-CDf, SEQ ID NO: 495), and R26G, V28G, A48R, and Y73C (e.g., TadA-CDg, SEQ ID NO: 496).

In some embodiments, the TadA-CD variants described above and herein may also comprises a V106W mutation.

In some embodiments, the TadA-CD variants comprise at least 80%, at least 85%, at least 90%, at least 95%, at least 98%, at least 99%, or at least 99.5% to any of the amino acid sequences of SEQ ID NOs: 489-496.

In some embodiments, the evolved TadA-Dual deaminase comprises the mutations R26G, V28A, N46I, A48R, Y73P, and H96N (TadA-CD-1, SEQ ID NO: 497) relative to the amino acid sequence of SEQ ID NO: 489. In some embodiments, the evolved TadA-Dual deaminase comprises the mutations R26G, V28A, N46T, A48R, Y73P, and H96N (TadA-CD-2, SEQ ID NO: 498) relative to the amino acid sequence of SEQ ID NO: 489. In some embodiments, the evolved TadA-Dual deaminase comprises the mutations R26G, V28A, N46T, A48R, Y73S, and H96N (TadA-CD-3, SEQ ID NO: 499) relative to the amino acid sequence of SEQ ID NO: 489. In some embodiments, the evolved TadA-Dual deaminase comprises the mutations R26G, V28A, N46V, A48R, Y73S, and H96N (TadA-CD-4, SEQ ID NO:500) relative to the amino acid sequence of SEQ ID NO: 489. In some embodiments, the evolved TadA-Dual deaminase comprises the mutations R26G, V28A, N46V, A48R, Y73P, and H96N (TadA-CD-5, SEQ ID NO: 501) relative to the amino acid sequence of SEQ ID NO: 489. In some embodiments, the evolved TadA-Dual deaminase comprises the mutations R26G, V28A, N46L, A48R, Y73P, and H96N (TadA-CD-6, SEQ ID NO: 502) relative to the amino acid sequence of SEQ ID NO: 489. In some embodiments, the evolved TadA-Dual deaminase comprises the mutations V28A, N46L, A48P, and Y73P (TadA-CD-7, SEQ ID NO: 503) relative to the amino acid sequence of SEQ ID NO: 489. In some embodiments, the evolved TadA-Dual deaminase comprises the mutations V28A, N46C, A48P, and Y73P (TadA-CD-8, SEQ ID NO: 504) relative to the amino acid sequence of SEQ ID NO: 489. In some embodiments, the evolved TadA-Dual deaminase comprises the mutations R26G, V28A, N46V, A48R, Y73P, and H96N (TadA-CD-9, SEQ ID NO: 505) relative to the amino acid sequence of SEQ ID NO: 489. In some embodiments, the evolved TadA-Dual deaminase comprises the mutations R26G, V28A, N46V, A48R, Q71H, Y73P, and H96N (TadA-CD-10, SEQ ID NO: 506) relative to the amino acid sequence of SEQ ID NO: 489. In some embodiments, the evolved TadA-Dual deaminase comprises the mutations R26G, V28A, N46L, A48R, Y73P, and H96N (TadA-CD-11, SEQ ID NO: 507) relative to the amino acid sequence of SEQ ID NO: 489. In some embodiments, the evolved TadA-Dual deaminase comprises the mutations R26G, V28A, N46C, A48R, Y73P, and H96N (TadA-CD-12, SEQ ID NO: 508) relative to the amino acid sequence of SEQ ID NO: 489. In some embodiments, the evolved TadA-Dual deaminase comprises the mutations R26G, V28A, N46C, A48R, Y73P, H96N, and A162V (TadA-CD-13, SEQ ID NO: 509) relative to the amino acid sequence of SEQ ID NO: 489.

In some embodiments, the evolved TadA-Dual deaminase comprises the mutations R26G, V28A, N46I, A48R, Y73S, and H96N (TadA-CD-14, SEQ ID NO: 510) relative to the amino acid sequence of SEQ ID NO: 489. In some embodiments, the evolved TadA-Dual deaminase comprises the mutations R26G, V28A, A48R, Q71S, Y73S, and H96N (TadA-CD-15, SEQ ID NO: 511) relative to the amino acid sequence of SEQ ID NO: 489. In some embodiments, the evolved TadA-Dual deaminase comprises the mutations R26G, V28A, N46L, A48R, and Y73P (TadA-CD-16, SEQ ID NO: 512) relative to the amino acid sequence of SEQ ID NO: 489. In some embodiments, the evolved TadA-Dual deaminase comprises the mutations R26G, V28A, N46L, A48R, Y73P, and H96N (TadA-CD-17, SEQ ID NO: 513) relative to the amino acid sequence of SEQ ID NO: 489. In some embodiments, the evolved TadA-Dual deaminase comprises the mutations R26G, V28A, Y73P, and H96N (TadA-CD-18, SEQ ID NO: 514) relative to the amino acid sequence of SEQ ID NO: 489. In some embodiments, the evolved TadA-Dual deaminase comprises the mutations R26G, V28A, N46V, A48R, Y73S, and H96N (TadA-CD-19, SEQ ID NO: 515) relative to the amino acid sequence of SEQ ID NO: 489. In some embodiments, the evolved TadA-Dual deaminase comprises the mutations R26G, V28A, N46V, A48R, Y73P, and H96N (TadA-CD-20, SEQ ID NO: 516) relative to the amino acid sequence of SEQ ID NO: 489. In some embodiments, the evolved TadA-Dual deaminase comprises the mutations R26G and N46L (TadA-CD-21, SEQ ID NO: 517) relative to the amino acid sequence of SEQ ID NO: 489. In some embodiments, the evolved TadA-Dual deaminase comprises the mutations R26G, V28A, N46I, A48R, Y73P, and H96N (TadA-CD-22, SEQ ID NO: 518) relative to the amino acid sequence of SEQ ID NO: 489. In some embodiments, the evolved TadA-Dual deaminase comprises the mutations R26G, V28A, N46V, A48R, Y73P, and H96N (TadA-CD-23, SEQ ID NO: 519) relative to the amino acid sequence of SEQ ID NO: 489. In some embodiments, the evolved TadA-Dual deaminase comprises the mutations R26G, V28A, A48P, Y73H, T79P, and H96N (TadA-CD-24, SEQ ID NO: 520) relative to the amino acid sequence of SEQ ID NO: 489. In some embodiments, the evolved TadA-Dual deaminase comprises the mutations R26G, N46I, and H96N (TadA-CD-25, SEQ ID NO: 521) relative to the amino acid sequence of SEQ ID NO: 489.

In some embodiments, the evolved TadA-Dual deaminase comprises the mutations R26G, V28A, N46V, A48R, Y73P, and H96N (TadA-CD-26, SEQ ID NO: 522) relative to the amino acid sequence of SEQ ID NO: 489. In some embodiments, the evolved TadA-Dual deaminase comprises the mutations R26G, V28A, N46L, A48R, Y73S, and H96N (TadA-CD-27, SEQ ID NO: 523) relative to the amino acid sequence of SEQ ID NO: 489. In some embodiments, the evolved TadA-Dual deaminase comprises the mutations R26G, V28A, N46C, A48R, H96N, and A162V (TadA-CD-28, SEQ ID NO: 524) relative to the amino acid sequence of SEQ ID NO: 489. In some embodiments, the evolved TadA-Dual deaminase comprises the mutations R26G, V28A, N46V, A48R, Q71H, Y73P, and H96N (TadA-CD-29, SEQ ID NO: 525) relative to the amino acid sequence of SEQ ID NO: 489. In some embodiments, the evolved TadA-Dual deaminase comprises the mutations R26G, V28A, N46C, A48R, Y73P, and H96N (TadA-CD-30, SEQ ID NO: 526) relative to the amino acid sequence of SEQ ID NO: 489. In some embodiments, the evolved TadA-Dual deaminase comprises the mutations R26G, V28A, N46C, A48R, Y73P, H96N, and A162V (TadA-CD-31, SEQ ID NO: 527) relative to the amino acid sequence of SEQ ID NO: 489.

In some embodiments, the evolved TadA-Dual deaminase comprises the mutations R26G, V28A, N46V, A48R, Y73P, and H96N (TadA-CD-32, SEQ ID NO: 528) relative to the amino acid sequence of SEQ ID NO: 489. In some embodiments, the evolved TadA-Dual deaminase comprises the mutations R26G, V28A, N46V, A48R, Y73S, and H96N (TadA-CD-33, SEQ ID NO: 529) relative to the amino acid sequence of SEQ ID NO: 489. In some embodiments, the evolved TadA-Dual deaminase comprises the mutations R26G, V28A, N46V, A48P, Y73S, and H96N (TadA-CD-34, SEQ ID NO: 530) relative to the amino acid sequence of SEQ ID NO: 489. In some embodiments, the evolved TadA-Dual deaminase comprises the mutations R26G, V28A, N46C, A48R, Y73P, and H96N (TadA-CD-35, SEQ ID NO: 531) relative to the amino acid sequence of SEQ ID NO: 489. In some embodiments, the evolved TadA-Dual deaminase comprises the mutations R26G, V28A, L34M, N46L, A48R, Y73P, and H96N (TadA-CD-36, SEQ ID NO: 532) relative to the amino acid sequence of SEQ ID NO: 489. In some embodiments, the evolved TadA-Dual deaminase comprises the mutations R26G, V28A, N46L, A48R, Y73P, and H96N (TadA-CD-37, SEQ ID NO: 533) relative to the amino acid sequence of SEQ ID NO: 489. In some embodiments, the evolved TadA-Dual deaminase comprises the mutations R26G, V28A, N46L, A48P, R64K, Y73P, and H96N (TadA-CD-38, SEQ ID NO: 534) relative to the amino acid sequence of SEQ ID NO: 489.

In some embodiments, the evolved TadA-Dual deaminase comprises the mutations N46I, S73P, and H154Q (TadA-CD-1, SEQ ID NO: 497) relative to the amino acid sequence of SEQ ID NO: 495. In some embodiments, the evolved TadA-Dual deaminase comprises the mutations N46T (TadA-CD-2, SEQ ID NO: 498) relative to the amino acid sequence of SEQ ID NO: 495. In some embodiments, the evolved TadA-Dual deaminase comprises the mutations N46T and H154Q (TadA-CD-3, SEQ ID NO: 499) relative to the amino acid sequence of SEQ ID NO: 495. In some embodiments, the evolved TadA-Dual deaminase comprises the mutations N46V and H154Q (TadA-CD-4, SEQ ID NO: 500) relative to the amino acid sequence of SEQ ID NO: 495. In some embodiments, the evolved TadA-Dual deaminase comprises the mutations N46V, S73P, G105S, and H154Q (TadA-CD-5, SEQ ID NO: 501) relative to the amino acid sequence of SEQ ID NO: 495. In some embodiments, the evolved TadA-Dual deaminase comprises the mutations N46L, S73P, and H154Q (TadA-CD-6, SEQ ID NO: 502) relative to the amino acid sequence of SEQ ID NO: 495. In some embodiments, the evolved TadA-Dual deaminase comprises the mutations G26R N46L, R48P, S73P, N96H, and H154Q (TadA-CD-7, SEQ ID NO: 503) relative to the amino acid sequence of SEQ ID NO: 495. In some embodiments, the evolved TadA-Dual deaminase comprises the mutations N46C, N96H, and H154Q (TadA-CD-8, SEQ ID NO: 504) relative to the amino acid sequence of SEQ ID NO: 495. In some embodiments, the evolved TadA-Dual deaminase comprises the mutations N46V, S73P, and H154Q (TadA-CD-9, SEQ ID NO: 505) relative to the amino acid sequence of SEQ ID NO: 495. In some embodiments, the evolved TadA-Dual deaminase comprises the mutations N46V, Q71H, S73P, and H154Q (TadA-CD-10, SEQ ID NO: 506) relative to the amino acid sequence of SEQ ID NO: 495. In some embodiments, the evolved TadA-Dual deaminase comprises the mutations N46L and H154Q (TadA-CD-11, SEQ ID NO: 507) relative to the amino acid sequence of SEQ ID NO: 495. In some embodiments, the evolved TadA-Dual deaminase comprises the mutations N46C, S73P, and H154Q (TadA-CD-12, SEQ ID NO: 508) relative to the amino acid sequence of SEQ ID NO: 495. In some embodiments, the evolved TadA-Dual deaminase comprises the mutations N46C, S73P, H154Q, and A162V (TadA-CD-13, SEQ ID NO: 509) relative to the amino acid sequence of SEQ ID NO: 495.

In some embodiments, the evolved TadA-Dual deaminase comprises the mutations N46I and H154Q (TadA-CD-14, SEQ ID NO: 510) relative to the amino acid sequence of SEQ ID NO: 495. In some embodiments, the evolved TadA-Dual deaminase comprises the mutations Q71S and H154Q (TadA-CD-15, SEQ ID NO: 511) relative to the amino acid sequence of SEQ ID NO: 489. In some embodiments, the evolved TadA-Dual deaminase comprises the mutations N46L, S73P, N79T, and N96H (TadA-CD-16, SEQ ID NO: 512) relative to the amino acid sequence of SEQ ID NO: 495. In some embodiments, the evolved TadA-Dual deaminase comprises the mutations N46L, S73P, N79T (TadA-CD-17, SEQ ID NO: 513) relative to the amino acid sequence of SEQ ID NO: 495. In some embodiments, the evolved TadA-Dual deaminase comprises the mutations R48A, S73P, and N79T (TadA-CD-18, SEQ ID NO: 514) relative to the amino acid sequence of SEQ ID NO: 495. In some embodiments, the evolved TadA-Dual deaminase comprises the mutations N46V and N79T (TadA-CD-19, SEQ ID NO: 515) relative to the amino acid sequence of SEQ ID NO: 495. In some embodiments, the evolved TadA-Dual deaminase comprises the mutations N46V, S73P, and N79T (TadA-CD-20, SEQ ID NO: 516) relative to the amino acid sequence of SEQ ID NO: 495. In some embodiments, the evolved TadA-Dual deaminase comprises the mutations A28V, N46L, R48A, S73Y, N79T, and N96H (TadA-CD-21, SEQ ID NO: 517) relative to the amino acid sequence of SEQ ID NO: 495. In some embodiments, the evolved TadA-Dual deaminase comprises the mutations N46I, S73P, and N79T (TadA-CD-22, SEQ ID NO: 518) relative to the amino acid sequence of SEQ ID NO: 495. In some embodiments, the evolved TadA-Dual deaminase comprises the mutations N46V, S73P, N79T, and G106S (TadA-CD-23, SEQ ID NO: 519) relative to the amino acid sequence of SEQ ID NO: 495. In some embodiments, the evolved TadA-Dual deaminase comprises the mutations R48P, S73H, and N79P (TadA-CD-24, SEQ ID NO: 520) relative to the amino acid sequence of SEQ ID NO: 495. In some embodiments, the evolved TadA-Dual deaminase comprises the mutations A28V, N46I, R48A, S73Y, and N79T (TadA-CD-25, SEQ ID NO: 521) relative to the amino acid sequence of SEQ ID NO: 495.

In some embodiments, the evolved TadA-Dual deaminase comprises the mutations N46V and S73P (TadA-CD-26, SEQ ID NO: 522) relative to the amino acid sequence of SEQ ID NO: 495. In some embodiments, the evolved TadA-Dual deaminase comprises the mutation N46L (TadA-CD-27, SEQ ID NO: 523) relative to the amino acid sequence of SEQ ID NO: 495. In some embodiments, the evolved TadA-Dual deaminase comprises the mutations N46C, S73Y, and A162V (TadA-CD-28, SEQ ID NO: 524) relative to the amino acid sequence of SEQ ID NO: 495. In some embodiments, the evolved TadA-Dual deaminase comprises the mutations N46V, Q71H, and S73P (TadA-CD-29, SEQ ID NO: 525) relative to the amino acid sequence of SEQ ID NO: 495. In some embodiments, the evolved TadA-Dual deaminase comprises the mutations N46C and S73P (TadA-CD-30, SEQ ID NO: 526) relative to the amino acid sequence of SEQ ID NO: 495. In some embodiments, the evolved TadA-Dual deaminase comprises the mutations N46C, S73P, and A162V (TadA-CD-31, SEQ ID NO: 527) relative to the amino acid sequence of SEQ ID NO: 495. In some embodiments, the evolved TadA-Dual deaminase comprises the mutations N46V and S73P (TadA-CD-32, SEQ ID NO: 528) relative to the amino acid sequence of SEQ ID NO: 495. In some embodiments, the evolved TadA-Dual deaminase comprises the mutation N46V (TadA-CD-33, SEQ ID NO: 529) relative to the amino acid sequence of SEQ ID NO: 495. In some embodiments, the evolved TadA-Dual deaminase comprises the mutations N46V and R48P (TadA-CD-34, SEQ ID NO: 530) relative to the amino acid sequence of SEQ ID NO: 495. In some embodiments, the evolved TadA-Dual deaminase comprises the mutations N46CV and S73P (TadA-CD-35, SEQ ID NO: 531) relative to the amino acid sequence of SEQ ID NO: 495. In some embodiments, the evolved TadA-Dual deaminase comprises the mutations L34M, N46L and S73P (TadA-CD-36, SEQ ID NO: 532) relative to the amino acid sequence of SEQ ID NO: 495. In some embodiments, the evolved TadA-Dual deaminase comprises the mutations N46L and S73P (TadA-CD-37, SEQ ID NO: 533) relative to the amino acid sequence of SEQ ID NO: 495. In some embodiments, the evolved TadA-Dual deaminase comprises the mutations N46L, r48P, R64K and S73P (TadA-CD-38, SEQ ID NO: 534) relative to the amino acid sequence of SEQ ID NO: 495.

In some embodiments, the TadA-CDs evolved from TadA-dual comprise at least 80%, 85%, 90%, 95%, 98%, 99%, or 99.5% identical to any of the amino acid sequences of SEQ ID NOs: 39, 41-54, and 359-383.

Exemplary TadA-derived cytosine base editor amino acid sequences include: TadA-CDa base editor (SpCas9n napDNAbp domain) (TadCBEa) (SEQ ID NO: 535), TadA-CDb base editor (SpCas9n napDNAbp domain) (TadCBEb) (SEQ ID NO: 536), TadA-CDc base editor (SpCas9n napDNAbp domain) (TadCBEc) (SEQ ID NO: 537), TadA-CDd base editor (SpCas9n napDNAbp domain) (TadCBEd) (SEQ ID NO: 538), TadA-CDe base editor (SpCas9n napDNAbp domain) (TadCBEe) (SEQ ID NO: 539), TadA-CDa (V106W) base editor (SpCas9n napDNAbp domain) (TadCBEa (V106W)) (SEQ ID NO: 540), TadA-CDd (V106W) base editor (SpCas9n napDNAbp domain) (TadCBEd (V106W)) (SEQ ID NO: 541), TadA-CDf base editor (SpCas9n napDNAbp domain) (TadCBEf) (SEQ ID NO: 542), TadA-CDg base editor (SpCas9n napDNAbp domain) (TadCBEg) (SEQ ID NO: 543), TadA-CDa: eNme2Cas9 base editor (SEQ ID NO: 544), TadA-CDa: SaCas9 base editor (SEQ ID NO: 545), TadA-CDa: SpCas9-NG base editor (SEQ ID NO: 546), TadA-CDa: enCjCas9 base editor (SEQ ID NO: 547).

Exemplary polynucleotides encoding TadA-derived cytosine base editors of the disclosure include: TadCBEa-eNme2-C-BE4max vector (SEQ ID NO: 548), TadCBEa-enCjCas9-BE4max vector (SEQ ID NO: 549), TadCBEa-SpCas9-BE4max vector (SEQ ID NO: 550), TadCBEa-SaCas9-BE4max vector (SEQ ID NO: 551), TadCBEa-SpCas9-NG-BE4max vector (SEQ ID NO: 552).

### Guide Polynucleotides

A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

In an embodiment, a guide polynucleotide described herein can be RNA or DNA. In one embodiment, the guide polynucleotide is a gRNA.

In some embodiments, the guide polynucleotide is at least one single guide RNA (“sgRNA” or “gRNA”). In some embodiments, a guide polynucleotide comprises two or more individual polynucleotides, which can interact with one another via for example complementary base pairing (e.g., a dual guide polynucleotide, dual gRNA). For example, a guide polynucleotide can comprise a CRISPR RNA (crRNA) and a trans-activating CRISPR RNA (tracrRNA) or can comprise one or more trans-activating CRISPR RNA (tracrRNA).

A guide polynucleotide may include natural or non-natural (or unnatural) nucleotides (e.g., peptide nucleic acid or nucleotide analogs). In some cases, the targeting region of a guide nucleic acid sequence (e.g., a spacer) can be at least 15, 16, 17, 18, 19, 20, 21, 22, 23, 24, 25, 26, 27, 28, 29, or 30 nucleotides in length.

In some embodiments, the methods described herein can utilize an engineered Cas protein. A guide RNA (gRNA) is a short synthetic RNA composed of a scaffold sequence necessary for Cas-binding and a user-defined ~20 nucleotide spacer that defines the genomic target to be modified. Exemplary gRNA scaffold sequences are provided in the sequence listing as SEQ ID NOs: 317-327 and 425. Thus, a skilled artisan can change the genomic target of the Cas protein specificity is partially determined by how specific the gRNA targeting sequence is for the genomic target compared to the rest of the genome. In embodiments, the spacer is about 10, 11, 12, 13, 14, 15, 16, 17, 18, 19, 20, 22, 23, 24, 25, or more nucleotides in length. The spacer of a gRNA can be or can be about 19, 20, or 21 nucleotides in length.

A gRNA or a guide polynucleotide can target any exon or intron of a gene target. In some embodiments, a composition comprises multiple gRNAs that all target the same exon or multiple gRNAs that target different exons. An exon and/or an intron of a gene can be targeted. A gRNA or a guide polynucleotide can target a nucleic acid sequence of about 20 nucleotides or less than about 20 nucleotides (e.g., at least about 5, 10, 15, 16, 17, 18, 19, 20, 21, 22, 23, 24, 25, 30 nucleotides), or anywhere between about 1-100 nucleotides (e.g., 5, 10, 15, 16, 17, 18, 19, 20, 21, 22, 23, 24, 25, 30, 40, 50, 60, 70, 80, 90, 100). A target nucleic acid sequence can be or can be about 20 bases immediately 5′ of the first nucleotide of the PAM. A gRNA can target a nucleic acid sequence. A target nucleic acid can be at least or at least about 1-10, 1-20, 1-30, 1-40, 1-50, 1-60, 1-70, 1-80, 1-90, or 1-100 nucleotides.

The guide polynucleotides can comprise standard ribonucleotides, modified ribonucleotides (e.g., pseudouridine), ribonucleotide isomers, and/or ribonucleotide analogs.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and may be separated by a direct repeat.

### Modified Polynucleotides

To enhance expression, stability, and/or genomic/base editing efficiency, and/or reduce possible toxicity, the base editor-coding sequence (e.g., mRNA) and/or the guide polynucleotide (e.g., gRNA) can be modified to include one or more modified nucleotides and/or chemical modifications, e.g. using pseudo-uridine, 5-Methyl-cytosine, 2′-O-methyl-3′-phosphonoacetate, 2′-O-methyl thioPACE (MSP), 2′-O-methyl-PACE (MP), 2′-fluoro RNA (2′-F-RNA), =constrained ethyl (S-cEt), 2′-O-methyl (‘M’), 2′-O-methyl-3′-phosphorothioate (‘MS’), 2′-O-methyl-3′-thiophosphonoacetate (‘MSP’), 5-methoxyuridine, phosphorothioate, and N1-Methylpseudouridine. Chemically protected gRNAs can enhance stability and editing efficiency in vivo and ex vivo. Methods for using chemically modified mRNAs and guide RNAs are known in the art and described, for example, by Jiang et al., Chemical modifications of adenine base editor mRNA and guide RNA expand its application scope. Nat Commun 11, 1979 (2020). doi.org/10.1038/s41467-020-15892-8, Callum et al., N1-Methylpseudouridine substitution enhances the performance of synthetic mRNA switches in cells, Nucleic Acids Research, Volume 48, Issue 6, 6 Apr. 2020, Page e35, and Andries et al., Journal of Controlled Release, Volume 217, 10 Nov. 2015, Pages 337-344, each of which is incorporated herein by reference in its entirety.

In some embodiments, the guide polynucleotide comprises one or more modified nucleotides at the 5′ end and/or the 3′ end of the guide. In some embodiments, the guide polynucleotide comprises two, three, four or more modified nucleosides at the 5′ end and/or the 3′ end of the guide. In some embodiments, the guide polynucleotide comprises two, three, four or more modified nucleosides at the 5′ end and/or the 3′ end of the guide.

In some embodiments, the guide comprises at least about 50%-75% modified nucleotides. In some embodiments, the guide comprises at least about 85% or more modified nucleotides. In some embodiments, at least about 1-5 nucleotides at the 5′ end of the gRNA are modified and at least about 1-5 nucleotides at the 3′ end of the gRNA are modified. In some embodiments, at least about 3-5 contiguous nucleotides at each of the 5′ and 3′ termini of the gRNA are modified. In some embodiments, at least about 20% of the nucleotides present in a direct repeat or anti-direct repeat are modified. In some embodiments, at least about 50% of the nucleotides present in a direct repeat or anti-direct repeat are modified. In some embodiments, at least about 50-75% of the nucleotides present in a direct repeat or anti-direct repeat are modified. In some embodiments, at least about 100 of the nucleotides present in a direct repeat or anti-direct repeat are modified. In some embodiments, at least about 20% or more of the nucleotides present in a hairpin present in the gRNA scaffold are modified. In some embodiments, at least about 50% or more of the nucleotides present in a hairpin present in the gRNA scaffold are modified. In some embodiments, the guide comprises a variable length spacer. In some embodiments, the guide comprises a 20-40 nucleotide spacer. In some embodiments, the guide comprises a spacer comprising at least about 20-25 nucleotides or at least about 30-35 nucleotides. In some embodiments, the spacer comprises modified nucleotides. In some embodiments, the guide comprises two or more of the following:
at least about 1-5 nucleotides at the 5′ end of the gRNA are modified and at least about 1-5 nucleotides at the 3′ end of the gRNA are modified;
at least about 20% of the nucleotides present in a direct repeat or anti-direct repeat are modified;
at least about 50-75% of the nucleotides present in a direct repeat or anti-direct repeat are modified;
at least about 20% or more of the nucleotides present in a hairpin present in the gRNA scaffold are modified;
a variable length spacer; and
a spacer comprising modified nucleotides.

In embodiments, the gRNA contains numerous modified nucleotides and/or chemical modifications. Such modifications can increase base editing ~2 fold in vivo or in vitro. In embodiments, the gRNA comprises 2′-O-methyl or phosphorothioate modifications. In an embodiment, the gRNA comprises 2′-O-methyl and phosphorothioate modifications. In an embodiment, the modifications increase base editing by at least about 2 fold.

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

A gRNA or a guide polynucleotide can also be modified by 5′ adenylate, 5′ guanosine-triphosphate cap, 5′ N7-Methylguanosine-triphosphate cap, 5′ triphosphate cap, 3′ phosphate, 3′ thiophosphate, 5′ phosphate, 5′ thiophosphate, Cis-Syn thymidine dimer, trimers, C12 spacer, C3 spacer, C6 spacer, dSpacer, PC spacer, rSpacer, Spacer 18, Spacer 9, 3′-3′ modifications, 2′-O-methyl thioPACE (MSP), 2′-O-methyl-PACE (MP), and constrained ethyl (S-cEt), 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′ DABCYL, black hole quencher 1, black hole quencher 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or 3′-end of a gRNA which can inhibit exonuclease degradation. In some cases, phosphorothioate bonds can be added throughout an entire gRNA to reduce attack by endonucleases.

### Fusion Proteins or Complexes Comprising a Nuclear Localization Sequence (NLS)

In some embodiments, the fusion proteins or complexes provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, the NLS is fused to the N-terminus or the C-terminus of the fusion protein. In some embodiments, the NLS is fused to the C-terminus or N-terminus of an nCas9 domain or a dCas9 domain. In some embodiments, the NLS is fused to the N-terminus or C-terminus of the Cas12 domain. In some embodiments, the NLS is fused to the N-terminus or C-terminus of the cytidine or adenosine deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences.

In some embodiments, the NLS is present in a linker or the NLS is flanked by linkers, for example described herein. A bipartite NLS comprises two basic amino acid clusters, which are separated by a relatively short spacer sequence (hence bipartite-2 parts, while monopartite NLSs are not). The NLS of nucleoplasmin, KR [PAATKKAGQA] KKKK (SEQ ID NO: 191), is the prototype of the ubiquitous bipartite signal: two clusters of basic amino acids, separated by a spacer of about 10 amino acids. The sequence of an exemplary bipartite NLS follows: PKKKRKVEGADKRTADGSEFESPKKKRKV (SEQ ID NO: 328).

In some embodiments, any of the fusion proteins or complexes provided herein comprise an NLS comprising the amino acid sequence EGADKRTADGSEFESPKKKRKV (amino acids 8 to 29 of SEQ ID NO 328). In some embodiments, any of the adenosine base editors provided herein comprise an NLS comprising the amino acid sequence EGADKRTADGSEFESPKKKRKV (amino acids 8 to 29 of SEQ ID NO: 328). In some embodiments, the NLS is at a C-terminal portion of the adenosine base editor. In some embodiments, the NLS is at the C-terminus of the adenosine base editor.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some embodiments, the additional domain can facilitate enzymatic or catalytic functions of the base editor, binding functions of the base editor, or be inhibitors of cellular machinery (e.g., enzymes) that could interfere with the desired base editing result. In some embodiments, a base editor comprises a nuclease, a nickase, a recombinase, a deaminase, a methyltransferase, a methylase, an acetylase, an acetyltransferase, a transcriptional activator, or a transcriptional repressor domain.

In some embodiments, a base editor comprises an uracil glycosylase inhibitor (UGI) domain. In some cases, a base editor is expressed in a cell in trans with a UGI polypeptide. In some embodiments, cellular DNA repair response to the presence of U:G heteroduplex DNA can be responsible for a reduction in nucleobase editing efficiency in cells. In such embodiments, uracil DNA glycosylase (UDG) can catalyze removal of U from DNA in cells, which can initiate base excision repair (BER), mostly resulting in reversion of the U:G pair to a C:G pair. In such embodiments, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein or complex comprising a UGI domain and/or a uracil stabilizing protein (USP) domain.

### Base Editor System

Provided herein are systems, compositions, and methods for editing a nucleobase using a base editor system. In some embodiments, the base editor system comprises (1) a base editor (BE) comprising a polynucleotide programmable nucleotide binding domain and a nucleobase editing domain (e.g., a deaminase domain) for editing the nucleobase; and (2) a guide polynucleotide (e.g., guide RNA) in conjunction with the polynucleotide programmable nucleotide binding domain. In some embodiments, the base editor system is a cytidine base editor (CBE) or an adenosine base editor (ABE). In some embodiments, the polynucleotide programmable nucleotide binding domain is a polynucleotide programmable DNA or RNA binding domain. In some embodiments, the nucleobase editing domain is a deaminase domain. In some embodiments, a deaminase domain can be a cytidine deaminase or an cytosine deaminase. In some embodiments, a deaminase domain can be an adenine deaminase or an adenosine deaminase. In some embodiments, the adenosine base editor can deaminate adenine in DNA. In some embodiments, the base editor is capable of deaminating a cytidine in DNA.

Use of the base editor system provided herein comprises the steps of: (a) contacting a target nucleotide sequence of a polynucleotide (e.g., double- or single stranded DNA or RNA) of a subject with a base editor system comprising a nucleobase editor (e.g., an adenosine base editor or a cytidine base editor) and a guide polynucleotide (e.g., gRNA), wherein the target nucleotide sequence comprises a targeted nucleobase pair; (b) inducing strand separation of said target region; (c) converting a first nucleobase of said target nucleobase pair in a single strand of the target region to a second nucleobase; and (d) cutting no more than one strand of said target region, where a third nucleobase complementary to the first nucleobase base is replaced by a fourth nucleobase complementary to the second nucleobase. It should be appreciated that in some embodiments, step (b) is omitted. In some embodiments, said targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

The components of a base editor system (e.g., a deaminase domain, a guide RNA, and/or a polynucleotide programmable nucleotide binding domain) may be associated with each other covalently or non-covalently. For example, in some embodiments, the deaminase domain can be targeted to a target nucleotide sequence by a polynucleotide programmable nucleotide binding domain, optionally where the polynucleotide programmable nucleotide binding domain is complexed with a polynucleotide (e.g., a guide RNA). In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component (e.g., the deaminase component) comprises an additional heterologous portion or domain that is capable of interacting with, associating with, or capable of forming a complex with a corresponding heterologous portion, antigen, or domain that is part of a polynucleotide programmable nucleotide binding domain and/or a guide polynucleotide (e.g., a guide RNA) complexed therewith. In some embodiments, the polynucleotide programmable nucleotide binding domain, and/or a guide polynucleotide (e.g., a guide RNA) complexed therewith, comprises an additional heterologous portion or domain that is capable of interacting with, associating with, or capable of forming a complex with a corresponding heterologous portion, antigen, or domain that is part of a nucleobase editing domain (e.g., the deaminase component). In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion is capable of binding to a polynucleotide linker. An additional heterologous portion may be a protein domain. In some embodiments, an additional heterologous portion comprises a polypeptide, such as a 22 amino acid RNA-binding domain of the lambda bacteriophage antiterminator protein N (N22p), a 2G12 IgG homodimer domain, an ABI, an antibody (e.g. an antibody that binds a component of the base editor system or a heterologous portion thereof) or fragment thereof (e.g. heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, an Fab2, miniantibodies, and/or ZIP antibodies), a barnase-barstar dimer domain, a Bcl-xL domain, a Calcineurin A (CAN) domain, a Cardiac phospholamban transmembrane pentamer domain, a collagen domain, a Com RNA binding protein domain (e.g. SfMu Com coat protein domain, and SfMu Com binding protein domain), a Cyclophilin-Fas fusion protein (CyP-Fas) domain, a Fab domain, an Fc domain, a fibritin foldon domain, an FK506 binding protein (FKBP) domain, an FKBP binding domain (FRB) domain of mTOR, a foldon domain, a fragment X domain, a GAI domain, a GID1 domain, a Glycophorin A transmembrane domain, a GyrB domain, a Halo tag, an HIV Gp41 trimerisation domain, an HPV45 oncoprotein E7 C-terminal dimer domain, a hydrophobic polypeptide, a K Homology (KH) domain, a Ku protein domain (e.g., a Ku heterodimer), a leucine zipper, a LOV domain, a mitochondrial antiviral-signaling protein CARD filament domain, an MS2 coat protein domain (MCP), a non-natural RNA aptamer ligand that binds a corresponding RNA motif/aptamer, a parathyroid hormone dimerization domain, a PP7 coat protein (PCP) domain, a PSD95-Dlgl-zo-1 (PDZ) domain, a PYL domain, a SNAP tag, a SpyCatcher moiety, a SpyTag moiety, a streptavidin domain, a streptavidin-binding protein domain, a streptavidin binding protein (SBP) domain, a telomerase Sm7 protein domain (e.g. Sm7 homoheptamer or a monomeric Sm-like protein), and/or fragments thereof. In embodiments, an additional heterologous portion comprises a polynucleotide (e.g., an RNA motif), such as an MS2 phage operator stem-loop (e.g., an MS2, an MS2 C-5 mutant, or an MS2 F-5 mutant), a non-natural RNA motif, a PP7 operator stem-loop, an SfMu phate Com stem-loop, a sterile alpha motif, a telomerase Ku binding motif, a telomerase Sm7 binding motif, and/or fragments thereof. Non-limiting examples of additional heterologous portions include polypeptides with at least about 85% sequence identity to any one or more of SEQ ID NOs: 380, 382, 384, 386-388, or fragments thereof. Non-limiting examples of additional heterologous portions include polynucleotides with at least about 85% sequence identity to any one or more of SEQ ID NOs: 379, 381, 383, 385, or fragments thereof.

In some instances, components of the base editing system are associated with one another through the interaction of leucine zipper domains (e.g., SEQ ID NOs: 387 and 388). In some cases, components of the base editing system are associated with one another through polypeptide domains (e.g., FokI domains) that associate to form protein complexes containing about, at least about, or no more than about 1, 2 (i.e., dimerize), 3, 4, 5, 6, 7, 8, 9, 10 polypeptide domain units, optionally the polypeptide domains may include alterations that reduce or eliminate an activity thereof.

In some instances, components of the base editing system are associated with one another through the interaction of multimeric antibodies or fragments thereof (e.g., IgG, IgD, IgA, IgM, IgE, a heavy chain domain 2 (CH₂) of IgM (MHD2) or IgE (EHD2), an immunoglobulin Fc region, a heavy chain domain 3 (CH3) of IgG or IgA, a heavy chain domain 4 (CH4) of IgM or IgE, an Fab, and an Fab2). In some instances, the antibodies are dimeric, trimeric, or tetrameric. In embodiments, the dimeric antibodies bind a polypeptide or polynucleotide component of the base editing system.

In some cases, components of the base editing system are associated with one another through the interaction of a polynucleotide-binding protein domain(s) with a polynucleotide(s). In some instances, components of the base editing system are associated with one another through the interaction of one or more polynucleotide-binding protein domains with polynucleotides that are self-complementary and/or complementary to one another so that complementary binding of the polynucleotides to one another brings into association their respective bound polynucleotide-binding protein domain(s).

In some instances, components of the base editing system are associated with one another through the interaction of a polypeptide domain(s) with a small molecule(s) (e.g., chemical inducers of dimerization (CIDs), also known as “dimerizers”). Non-limiting examples of CIDs include those disclosed in Amara, et al., “A versatile synthetic dimerizer for the regulation of protein-protein interactions,” PNAS, 94:10618-10623 (1997); and Voß, et al. “Chemically induced dimerization: reversible and spatiotemporal control of protein function in cells,” Current Opinion in Chemical Biology, 28:194-201 (2015), the disclosures of each of which are incorporated herein by reference in their entireties for all purposes. In some embodiments, the base editor inhibits base excision repair (BER) of the edited strand. In some embodiments, the base editor protects or binds the non-edited strand. In some embodiments, the base editor comprises UGI activity or USP activity. In some embodiments, the base editor comprises a catalytically inactive inosine-specific nuclease.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Protein domains included in the fusion protein can be a heterologous functional domain. Non-limiting examples of protein domains which can be included in the fusion protein include a deaminase domain (e.g., cytidine deaminase and/or adenosine deaminase), a uracil glycosylase inhibitor (UGI) domain, epitope tags, and reporter gene sequences.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the base editor is ABE8.1, which comprises or consists essentially of the following sequence or a fragment thereof having adenosine deaminase activity: SEQ ID NO: 331. Other ABE8 sequences are provided in the attached sequence listing (SEQ ID NOs: 332-354).

In some embodiments, the base editor includes an adenosine deaminase variant comprising an amino acid sequence, which contains alterations relative to an ABE 7*10 reference sequence, as described herein. The term “monomer” as used in Table 7 refers to a monomeric form of TadA*7.10 comprising the alterations described. The term “heterodimer” as used in Table 7 refers to the specified wild-type E. coli TadA adenosine deaminase fused to a TadA*7.10 comprising the alterations as described.

TABLE 7
Adenosine Deaminase Base Editor Variants

| | Adenosine | |
|---|---|---|
| ABE | Deaminase | Adenosine Deaminase Description |
| ABE-605m | MSP605 | monomer_TadA*7.10 + V82G + Y147T + Q154S |
| ABE-680m | MSP680 | monomer_TadA*7.10 + I76Y + V82G + Y147T + Q154S |
| ABE-823m | MSP823 | monomer_TadA*7.10 + L36H + V82G + Y147T + Q154S + |
| | | N157K |
| ABE-824m | MSP824 | monomer_TadA*7.10 + V82G + Y147D + F149Y + Q154S + |
| | | D167N |
| ABE-825m | MSP825 | monomer_TadA*7.10+ L36H + V82G + Y147D + F149Y + |
| | | Q154S + N157K + D167N |
| ABE-827m | MSP827 | monomer_TadA*7.10 + L36H + I76Y + V82G + Y147T + Q154S + |
| | | N157K |
| ABE-828m | MSP828 | monomer_TadA*7.10 + I76Y + V82G + Y147D + F149Y + Q154S + |
| | | D167N |
| ABE-829m | MSP829 | monomer_TadA*7.10 + L36H + I76Y + V82G + Y147D + F149Y + |
| | | Q154S + N157K + D167N |
| ABE-605d | MSP605 | heterodimer_(WT) + (TadA*7.10 + V82G + Y147T + Q154S) |
| ABE-680d | MSP680 | heterodimer_(WT) + (TadA*7.10 + I76Y + V82G + Y147T + |
| | | Q154S) |
| ABE-823d | MSP823 | heterodimer_(WT) + (TadA*7.10 + L36H + V82G + Y147T + |
| | | Q154S + N157K) |
| ABE-824d | MSP824 | heterodimer_(WT) + (TadA*7.10 + V82G + Y147D + F149Y + |
| | | Q154S + D167N) |
| ABE-825d | MSP825 | heterodimer_(WT) + (TadA*7.10 + L36H + V82G + Y147D + |
| | | F149Y + Q154S + N157K + D167N) |
| ABE-827d | MSP827 | heterodimer_(WT) + (TadA*7.10 + L36H + I76Y + V82G + Y147T + |
| | | Q154S + N157K) |
| ABE-828d | MSP828 | heterodimer_(WT) + (TadA*7.10 + I76Y + V82G + Y147D + |
| | | F149Y + Q154S + D167N) |
| ABE-829d | MSP829 | heterodimer_(WT) + (TadA*7.10 + L36H + I76Y + V82G + Y147D + |
| | | F149Y + Q154S + N157K + D167N) |

In some embodiments, the base editor comprises a domain comprising all or a portion (e.g., a functional portion) of a uracil glycosylase inhibitor (UGI) or a uracil stabilizing protein (USP) domain.

### Linkers

In certain embodiments, linkers may be used to link any of the peptides or peptide domains of the disclosure. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.).

In some embodiments, any of the fusion proteins provided herein, comprise a cytidine or adenosine deaminase and a Cas9 domain that are fused to each other via a linker. Various linker lengths and flexibilities between the cytidine or adenosine deaminase and the Cas9 domain can be employed (e.g., ranging from very flexible linkers of the form (GGGS)ₙ (SEQ ID NO: 246), (GGGGS)ₙ (SEQ ID NO: 247), and (G)ₙ to more rigid linkers of the form (EAAAK)ₙ (SEQ ID NO: 248), (SGGS)ₙ (SEQ ID NO: 355), SGSETPGTSESATPES (SEQ ID NO: 249) (see, e.g., Guilinger J P, et al. Fusion of catalytically inactive Cas9 to FokI nuclease improves the specificity of genome modification. Nat. Biotechnol. 2014; 32 (6): 577-82; the entire contents are incorporated herein by reference) and (XP) n) in order to achieve the optimal length for activity for the cytidine or adenosine deaminase nucleobase editor. In some embodiments, n is 1, 2, 3, 4, 5, 6, 7, 8, 9, 10, 11, 12, 13, 14, or 15. In some embodiments, the linker comprises a (GGS) n motif, wherein n is 1, 3, or 7. In some embodiments, cytidine deaminase or adenosine deaminase and the Cas9 domain of any of the fusion proteins provided herein are fused via a linker comprising the amino acid sequence SGSETPGTSESATPES (SEQ ID NO: 249), which can also be referred to as the XTEN linker.

In some embodiments, the domains of the base editor are fused via a linker that comprises the amino acid sequence of:

(SEQ ID NO: 356)
SGGSSGSETPGTSESATPESSGGS,
(SEQ ID NO: 357)
SGGSSGGSSGSETPGTSESATPESSGGSSGGS,
(SEQ ID NO: 358)
GGSGGSPGSPAGSPTSTEEGTSESATPESGPGTSTEPSEGSAPGSPA
GSPTSTEEGTSTEPSEGSAPGTSTEPSEGSAPGTSESATPESGPGSE
PATSGGSGGS,
(SEQ ID NO: 468)
EGGSEEEEESGS,
or
(SEQ ID NO: 469)
KGPKPKKEESEK

In some embodiments, domains of the base editor are fused via a linker comprising the amino acid sequence SGSETPGTSESATPES (SEQ ID NO: 249), which may also be referred to as the XTEN linker. In some embodiments, a linker comprises the amino acid sequence SGGS (SEQ ID NO: 355). In some embodiments, the linker is 24 amino acids in length. In some embodiments, the linker comprises the amino acid sequence SGGSSGGSSGSETPGTSESATPES (SEQ ID NO: 359). In some embodiments, the linker is 40 amino acids in length. In some embodiments, the linker comprises the amino acid sequence: SGGSSGGSSGSETPGTSESATPESSGGSSGGSSGGSSGGS (SEQ ID NO: 360). In some embodiments, the linker is 64 amino acids in length. In some embodiments, the linker comprises the amino acid sequence: SGGSSGGSSGSETPGTSESATPESSGGSSGGSSGGSSGGSSGSETPGTSESATPESSGGSSGGS (SEQ ID NO: 361). In some embodiments, the linker is 92 amino acids in length. In some embodiments, the linker comprises the amino acid sequence:

(SEQ ID NO: 362)
PGSPAGSPTSTEEGTSESATPESGPGTSTEPSEGSAPGSPAGSPTST
EEGTSTEPSEGSAPGTSTEPSEGSAPGTSESATPESGPGSEPATS

In some embodiments, a linker comprises a plurality of proline residues and is 5-21, 5-14, 5-9, 5-7 amino acids in length, e.g., PAPAP (SEQ ID NO: 363), PAPAPA (SEQ ID NO: 364), PAPAPAP (SEQ ID NO: 365), PAPAPAPA (SEQ ID NO: 366), P(AP)4 (SEQ ID NO: 367), P(AP)7 (SEQ ID NO: 368), P(AP)10 (SEQ ID NO: 369) (see, e.g., Tan J, Zhang F, Karcher D, Bock R. Engineering of high-precision base editors for site-specific single nucleotide replacement. Nat Commun. 2019 Jan. 25; 10 (1): 439; the entire contents are incorporated herein by reference). Such proline-rich linkers are also termed “rigid” linkers.

Nucleic Acid Programmable DNA Binding Proteins with Guide RNAs

Provided herein are compositions and methods for base editing in cells. Further provided herein are compositions comprising a guide polynucleotide sequence, e.g., a guide RNA sequence, or a combination of 2, 3, 4, 5, 6, 7, 8, 9, 10, 11, 12, 13, 14, 15, 16, 17, 18, 19, 20, or more guide RNAs as provided herein. In some embodiments, a composition for base editing as provided herein further comprises a polynucleotide that encodes a base editor, e.g., a C-base editor or an A-base editor. For example, a composition for base editing may comprise a mRNA sequence encoding a BE, a BE4, an ABE, and a combination of one or more guide RNAs as provided. A composition for base editing may comprise a base editor polypeptide and a combination of one or more of any guide RNAs provided herein. Such a composition may be used to effect base editing in a cell through different delivery approaches, for example, electroporation, nucleofection, viral transduction or transfection. In some embodiments, the composition for base editing comprises an mRNA sequence that encodes a base editor and a combination of one or more guide RNA sequences provided herein for electroporation.

Some aspects of this disclosure provide systems comprising any of the fusion proteins or complexes provided herein, and a guide RNA bound to a nucleic acid programmable DNA binding protein (napDNAbp) domain (e.g., a Cas9 (e.g., a dCas9, a nuclease active Cas9, or a Cas9 nickase) or Cas12) of the fusion protein or complex. These complexes are also termed ribonucleoproteins (RNPs). In some embodiments, the guide nucleic acid (e.g., guide RNA) is from 15-100 nucleotides long and comprises a sequence of at least 10 contiguous nucleotides that is complementary to a target sequence. In some embodiments, the target sequence is a DNA sequence. In some embodiments, the target sequence is an RNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 3 or 5′-NAA-3′). In some embodiments, the guide nucleic acid (e.g., guide RNA) is complementary to a sequence in a gene of interest (e.g., a gene associated with a disease or disorder). Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA, wherein the guide RNA is about 15-100 nucleotides long and comprises a sequence of at least 10 contiguous nucleotides that is complementary to a target sequence.

The domains of the base editor disclosed herein can be arranged in any order.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence.

### Methods of Using Fusion Proteins or Complexes Comprising a Cytidine or Adenosine Deaminase and a Cas9 Domain

Some aspects of this disclosure provide methods of using the fusion proteins, or complexes provided herein. For example, some aspects of this disclosure provide methods comprising contacting a DNA molecule with any of the fusion proteins or complexes provided herein, and with at least one guide RNA described herein.

In some embodiments, a fusion protein or complex of the disclosure is used for editing a target gene of interest. In particular, a cytidine deaminase or adenosine deaminase nucleobase editor described herein is capable of making multiple mutations within a target sequence. These mutations may affect the function of the target. For example, when a cytidine deaminase or adenosine deaminase nucleobase editor is used to target a regulatory region the function of the regulatory region is altered and the expression of the downstream protein is reduced or eliminated.

### Base Editor Efficiency

In some embodiments, the purpose of the methods provided herein is to alter a gene and/or gene product via gene editing. The nucleobase editing proteins provided herein can be used for gene editing-based human therapeutics in vitro or in vivo. It will be understood by the skilled artisan that the nucleobase editing proteins provided herein, e.g., the fusion proteins or complexes comprising a polynucleotide programmable nucleotide binding domain (e.g., Cas9) and a nucleobase editing domain (e.g., an adenosine deaminase domain or a cytidine deaminase domain) can be used to edit a nucleotide from A to G or C to T.

Advantageously, base editing systems as provided herein provide genome editing without generating double-strand DNA breaks, without requiring a donor DNA template, and without inducing an excess of stochastic insertions and deletions as CRISPR may do. In some embodiments, the present disclosure provides base editors that efficiently generate an intended mutation, such as a STOP codon, in a nucleic acid (e.g., a nucleic acid within a genome of a subject) without generating a significant number of unintended mutations, such as unintended point mutations.

The base editors of the disclosure advantageously modify a specific nucleotide base encoding a protein without generating a significant proportion of indels (i.e., insertions or deletions). Such indels can lead to frame shift mutations within a coding region of a gene.

In some embodiments, the base editors provided herein are capable of generating a ratio of intended mutations to indels (i.e., intended point mutations:unintended point mutations) that is greater than 1:1. In some embodiments, the base editors provided herein are capable of generating a ratio of intended mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more. The number of intended mutations and indels may be determined using any suitable method.

In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein can limit the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%.

Base editing is often referred to as a “modification”, such as, a genetic modification, a gene modification and modification of the nucleic acid sequence and is clearly understandable based on the context that the modification is a base editing modification. A base editing modification is therefore a modification at the nucleotide base level, for example as a result of the deaminase activity discussed throughout the disclosure, which then results in a change in the gene sequence and may affect the gene product.

In some embodiments, the modification, e.g., single base edit results in about or at least about a 10%, 15%, 20%, 25%, 30%, 35%, 40%, 45%, 50%, 55%, 60%, 65%, 70%, 75%, 80%, 85%, 90%, 95%, 99%, or 100% reduction, or reduction to an undetectable level, of the gene targeted expression.

The disclosure provides adenosine deaminase variants (e.g., ABE8 variants) that have increased efficiency and specificity. In particular, the adenosine deaminase variants described herein are more likely to edit a desired base within a polynucleotide and are less likely to edit bases that are not intended to be altered (e.g., “bystanders”).

In some embodiments, any of the base editing system comprising one of the ABE8 base editor variants described herein has reduced bystander editing or mutations by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or 99% compared to a base editor system comprising an ABE7 base editor, e.g., ABE7.10.

In some embodiments, any of the ABE8 base editor variants described herein has higher base editing efficiency compared to the ABE7 base editors. In some embodiments, any of the ABE8 base editor variants described herein have at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, 99%, 100%, 105%, 110%, 115%, 120%, 125%, 130%, 135%, 140%, 145%, 150%, 155%, 160%, 165%, 170%, 175%, 180%, 185%, 190%, 195%, 200%, 210%, 220%, 230%, 240%, 250%, 260%, 270%, 280%, 290%, 300%, 310%, 320%, 330%, 340%, 350%, 360%, 370%, 380%, 390%, 400%, 450%, or 500% higher base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA.

In some embodiments, the method described herein, for example, the base editing methods has minimum to no off-target effects. In some embodiments, the method described herein, for example, the base editing methods, has minimal to no chromosomal translocations.

In some embodiments, the base editing method described herein results in about 91%, 92%, 93%, 94%, 95%, 96%, 97%, 98%, 99% or 100% of a cell population that have been successfully edited.

In some embodiments, the percent of viable cells in a cell population following a base editing intervention is greater than at least 60%, 70%, 80%, or 90% of the starting cell population at the time of the base editing event. In some embodiments, the percent of viable cells in a cell population following editing is about 70%. In some embodiments, the percent of viable cells in a cell population following editing is about 75%. In some embodiments, the percent of viable cells in a cell population following editing is about 80%. In some embodiments, the percent of viable cells in a cell population as described above is about 85%. In some embodiments, the percent of viable cells in a cell population as described above is about 90%, or about 91%, 92%, 93%, 94% 95%, 96%, 97%, 98%, 99%, or 100% of the cells in the population at the time of the base editing event.

In embodiments, the cell population is a population of cells contacted with a base editor, complex, or base editor system of the present disclosure.

The number of intended mutations and indels can be determined using any suitable method, for example, as described in International PCT Application Nos. PCT/US2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3: eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

### Multiplex Editing

In some embodiments, the base editor system provided herein is capable of multiplex editing of a plurality of nucleobase pairs in one or more genes or polynucleotide sequences. In some embodiments, the plurality of nucleobase pairs is located in the same gene or in one or more genes, wherein at least one gene is located in a different locus. In some embodiments, the multiplex editing comprises one or more guide polynucleotides. In some embodiments, the multiplex editing comprises one or more base editor systems. In some embodiments, the multiplex editing comprises one or more base editor systems with a single guide polynucleotide or a plurality of guide polynucleotides. In some embodiments, the multiplex editing comprises one or more guide polynucleotides with a single base editor system. It should be appreciated that the characteristics of the multiplex editing using any of the base editors as described herein can be applied to any combination of methods using any base editor provided herein. It should also be appreciated that the multiplex editing using any of the base editors as described herein can comprise a sequential editing of a plurality of nucleobase pairs.

In some embodiments, the base editor system capable of multiplex editing of a plurality of nucleobase pairs in one or more genes comprises one of ABE7, ABE8, and/or ABE9 base editors.

### Expression of Fusion Proteins or Complexes in a Host Cell

Fusion proteins or complexes of the disclosure comprising a deaminase may be expressed in virtually any host cell of interest, including but not limited to bacteria, yeast, fungi, insects, plants, and animal cells using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal, ligated with a DNA encoding one or more additional components of a base editing system. The base editing system is translated in a host cell to form a complex.

A polynucleotide encoding a polypeptide described herein can be obtained by chemically synthesizing the polynucleotide, or by connecting synthesized partly overlapping oligo short chains by utilizing the PCR method and the Gibson Assembly method to construct a polynucleotide (e.g., DNA) encoding the full length thereof. The advantage of constructing a full-length polynucleotide by chemical synthesis or a combination of PCR method or Gibson Assembly method is that the codons to be used can be selected in according to the host into which the polynucleotide is to be introduced. In the expression from a heterologous DNA molecule, the protein expression level is expected to increase by converting the DNA sequence thereof to a codon highly frequently used in the host organism. Codon use data for a host cell (e.g., codon use data available at kazusa. or .jp/codon/index.html) can be used to guide codon optimization for a polynucleotide sequence encoding a polypeptide. Codons having low use frequency in the host may be converted to a codon coding the same amino acid and having high use frequency.

An expression vector containing a polynucleotide encoding a nucleic acid sequence-recognizing module and/or a nucleic acid base converting enzyme can be produced, for example, by linking the DNA to the downstream of a promoter in a suitable expression vector.

As the expression vector, Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as .lambda phage and the like; insect virus vectors such as baculovirus and the like (e.g., BmNPV, AcNPV); animal virus vectors such as retrovirus, vaccinia virus, adenovirus and the like, and the like are used.

Regarding the promoter to be used, any promoter appropriate for a host to be used for gene expression can be used. In a method using double-stranded breaks, since the survival rate of the host cell sometimes reduces markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitutive promoter can be used without limitation.

For example, when the host is an animal cell, an SR.alpha. promoter, SV40 promoter, LTR promoter, cytomegalovirus (CMV) promoter, Rous sarcoma virus (RSV) promoter, Moloney mouse leukemia virus (MoMuLV), LTR, herpes simplex virus thymidine kinase (HSV-TK) promoter, and the like can be used. Of these, CMV promoter, SR.alpha. promoter and the like may be used.

Expression vectors for use in the present disclosure, besides those mentioned above, can comprise an enhancer, a splicing signal, a terminator, a poly A addition signal, a selection marker such as drug resistance gene, an auxotrophic complementary gene and the like, a replication origin, and the like can be used.

An RNA encoding a protein domain described herein can be prepared by, for example, in vitro transcription of a nucleic acid sequence encoding any of the fusion proteins or complexes disclosed herein.

A fusion protein or complex of the disclosure can be intracellularly expressed by introducing into the cell an expression vector comprising a nucleic acid sequence encoding the fusion protein or complex.

An expression vector can be introduced by a known method (e.g., the lysozyme method, the competent method, the PEG method, the CaCl₂) coprecipitation method, electroporation, microinjection, particle gun method, lipofection, Agrobacterium-mediated delivery, etc.) according to the kind of the host.

A vector can be introduced into an animal cell according to the methods described in, for example, Cell Engineering additional volume 8, New Cell Engineering Experiment Protocol, 263-267 (1995) (published by Shujunsha), and Virology, 52, 456 (1973).

A cell comprising a vector can be cultured according to a known method according to the kind of the host.

As a medium for culturing an animal cell, for example, minimum essential medium (MEM) containing about 5 to about 20% of fetal bovine serum [Science, 122, 501 (1952)], Dulbecco's modified Eagle medium (DMEM) [Virology, 8, 396 (1959)], RPMI 1640 medium [The Journal of the American Medical Association, 199, 519 (1967)], 199 medium [Proceeding of the Society for the Biological Medicine, 73, 1 (1950)] and the like are used. The pH of the medium may be between about 6 to about 8. The culture is performed at generally about 30° C. to about 40° C. Where necessary, aeration and stirring may be performed.

When a higher eukaryotic cell, such as animal cell, insect cell, plant cell and the like is used as a host cell, a polynucleotide encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Alternatively, an inductive promoter can also be utilized as a vector removal mechanism when higher eukaryotic cells, such as animal cell, insect cell, plant cell and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicable in the presence of an induction substance, when the induction substance is removed, autonomous replication is not available, and the vector naturally falls off along with cell division (autonomous replication is not possible by the addition of tetracycline and doxycycline in Tet-OFF system vector).

### Delivery Systems

### Nucleic Acid-Based Delivery of Base Editor Systems

Nucleic acid molecules encoding a base editor system according to the present disclosure can be administered to subjects or delivered into cells in vitro or in vivo by art-known methods or as described herein. For example, a base editor system comprising a deaminase (e.g., cytidine or adenine deaminase) can be delivered by vectors (e.g., viral or non-viral vectors), or by naked DNA, DNA complexes, lipid nanoparticles, or a combination of the aforementioned compositions. A base editor system may be delivered to a cell using any methods available in the art including, but not limited to, physical methods (e.g., electroporation, particle gun, calcium phosphate transfection), viral methods, non-viral methods (e.g., liposomes, cationic methods, lipid nanoparticles, polymeric nanoparticles), or biological non-viral methods (e.g., attenuated bacterial, engineered bacteriophages, mammalian virus-like particles, biological liposomes, erythrocyte ghosts, exosomes).

Nanoparticles, which can be organic or inorganic, are useful for delivering a base editor system or component thereof. Nanoparticles are well known in the art and any suitable nanoparticle can be used to deliver a base editor system or component thereof, or a nucleic acid molecule encoding such components. In one example, organic (e.g., lipid and/or polymer) nanoparticles are suitable for use as delivery vehicles in certain embodiments of this disclosure. Non-limiting examples of lipid nanoparticles suitable for use in the methods of the present disclosure include those described in International Patent Application Publications No. WO2022140239, WO2022140252, WO2022140238, WO2022159421, WO2022159472, WO2022159475, WO2022159463, WO2021113365, WO2024019936, and WO2021141969, the disclosures of each of which is incorporated herein by reference in its entirety for all purposes.

### Viral Vectors

A base editor described herein can be delivered with a viral vector. In some embodiments, a base editor disclosed herein can be encoded on a nucleic acid that is contained in a viral vector. In some embodiments, one or more components of the base editor system can be encoded on one or more viral vectors.

Viral vectors can include lentivirus (e.g., HIV and FIV-based vectors), Adenovirus (e.g., AD100), Retrovirus (e.g., Maloney murine leukemia virus, MML-V), herpesvirus vectors (e.g., HSV-2), rabies virus (see, e.g., U.S. Patent Application Publication No. US 2022/0290164 A1, the disclosure of which is incorporated herein by reference in its entirety for all purposes), and Adeno-associated viruses (AAVs), or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g., a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

Viral vectors can be selected based on the application. For example, for in vivo gene delivery, AAV can be advantageous over other viral vectors. In some embodiments, AAV allows low toxicity, which can be due to the purification method not requiring ultra-centrifugation of cell particles that can activate the immune response. In some embodiments, AAV allows low probability of causing insertional mutagenesis because it doesn't integrate into the host genome. Adenoviruses are commonly used as vaccines because of the strong immunogenic response they induce. Packaging capacity of the viral vectors can limit the size of the base editor that can be packaged into the vector.

AAV has a packaging capacity of about 4.5 Kb or 4.75 Kb including two 145 base inverted terminal repeats (ITRs). This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some embodiments, the disclosed base editors are 4.5 kb or less in length.

An AAV can be AAV1, AAV2, AAV5, AAV6, AAV9, PHP.EB, PHP.B, AAV.CAP-B10, AAV, CAP-B22, AAV-rh10, a PAL family AAV, or any combination thereof. In embodiments, the AAV is capable of crossing the blood-brain barrier (see, e.g., those AAV vectors disclosed in Liu, et al. “Crossing the blood-brain barrier with AAV vectors,” Metabolic Brain Disease, 36:45-52 (2021), the disclosure of which is incorporated herein by reference in its entirety for all purposes). One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82:5887-5911 (2008)).

In some embodiments, the AAV vector contains a PAL family AAV capsid (see, Stanton, A., et al. Med 4:31-50 (2023) (doi: doi.org/10.1016/j.medj.2022.11.002), the disclosure of which is incorporated herein by reference in its entirety for all purposes). In some embodiments, lentiviral vectors are used to transduce a cell of interest with a polynucleotide encoding a base editor or base editor system as provided herein. Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

In another embodiment, minimal non-primate lentiviral vectors based on the equine infectious anemia virus (EIAV) are also contemplated. In another embodiment, RetinoStat®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors are contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-poly A tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

### Non-Viral Platforms for Gene Transfer

Non-viral platforms for introducing a heterologous polynucleotide into a cell of interest are known in the art.

For example, the disclosure provides a method of inserting a heterologous polynucleotide into the genome of a cell using a Cas9 or Cas12 (e.g., Cas12b) ribonucleoprotein complex (RNP)-DNA template complex where an RNP including a Cas9 or Cas12 nuclease domain and a guide RNA, wherein the guide RNA specifically hybridizes to a target region of the genome of the cell, and wherein the Cas nuclease domain cleaves the target region to create an insertion site in the genome of the cell. A DNA template is then used to introduce a heterologous polynucleotide. In embodiments, the DNA template is a double-stranded or single-stranded DNA template, wherein the size of the DNA template is about 200 nucleotides or is greater than about 200 nucleotides, wherein the 5′ and 3′ ends of the DNA template comprise nucleotide sequences that are homologous to genomic sequences flanking the insertion site. In some embodiments, the DNA template is a single-stranded circular DNA template. In embodiments, the molar ratio of RNP to DNA template in the complex is from about 3:1 to about 100:1.

In some embodiments, the DNA template is a linear DNA template. In some examples, the DNA template is a single-stranded DNA template. In certain embodiments, the single-stranded DNA template is a pure single-stranded DNA template. In some embodiments, the single stranded DNA template is a single-stranded oligodeoxynucleotide (ssODN).

In other embodiments, a single-stranded DNA (ssDNA) can produce efficient homology-directed repair (HDR) with minimal off-target integration. In one embodiment, an ssDNA phage is used to efficiently and inexpensively produce long circular ssDNA (cssDNA) donors. These cssDNA donors serve as efficient HDR templates when used with Cas9 or Cas12 (e.g., Cas12a, Cas12b), with integration frequencies superior to linear ssDNA (IssDNA) donors.

In some embodiments, a heterologous polynucleotide may be inserted into the genome of a cell using a transposable element such as a transposon, as described, for example, in Tipanee, et al. Human Gene Therapy, November 2017, 1087-1104, DOI: 10.1089/hum.2017.128. Transposable elements are divided into two categories: retrotransposons and DNA transposons. Transposable elements can alter the genome of the host cells through insertions, duplications, deletions, and translocations. Retrotransposons are described as mobile elements that employ an RNA intermediate that is first reverse transcribed into a complementary single-stranded (c) DNA strand by a reverse transcriptase encoded by the retrotransposon. Subsequently, the single-stranded DNA is converted into a double-stranded DNA that then integrates into the host genome. This so-called “replicative mechanism” yields several new copies of retrotransposons expanding throughout the target genome over evolutionary time. Retrotransposons are categorized into many subtypes according to the DNA sequences of the long terminal repeats and its open reading frames. Retrotransposons were employed to enable transgene integration into the target cell DNA, in some cases relying on adenoviral delivery. Alternatively, DNA transposons translocate via a “non-replicative mechanism,” whereby two Terminal Inverted Repeats (TIRs) are recognized and cleaved by a transposase enzyme, releasing the cognate DNA transposons with free DNA ends. The excised DNA transposons then integrate into a new genomic region where target sites are recognized and cut by the same transposase. This cut-and-paste mechanism usually duplicates DNA target sites upon insertion, leaving target site duplications (TSDs). Non-limiting examples of transposons include the Sleeping Beauty (SB) transposon, the piggyBac (PB) transposon, and Tol2 transposable elements.

### Inteins

Inteins (intervening protein) are auto-processing domains found in a variety of diverse organisms, which carry out a process known as protein splicing.

Non-limiting examples of inteins include any intein or intein-pair known in the art, which include a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138 (7): 2162-5, incorporated herein by reference), and DnaE. Non-limiting examples of pairs of inteins that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference). Exemplary nucleotide and amino acid sequences of inteins are provided in the Sequence Listing at SEQ ID NOs: 370-377 and 389-424. Inteins suitable for use in embodiments of the present disclosure and methods for use thereof are described in U.S. Pat. No. 10,526,401, International Patent Application Publications No. WO 2013/045632, WO 2024/073385, or WO 2020/051561, and in U.S. Patent Application Publication No. US 2020/0055900, the full disclosures of which are incorporated herein by reference in their entireties by reference for all purposes.

Intein-N and intein-C may be fused to the N-terminal portion of a split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N-[N-terminal portion of the split Cas9]-[intein-N]-C. In some embodiments, an intein-C is fused to the N-terminus of the C-terminal portion of the split Cas9, i.e., to form a structure of N-[intein-C]-[C-terminal portion of the split Cas9]-C. In embodiments, a base editor is encoded by two polynucleotides, where one polynucleotide encodes a fragment of the base editor fused to an intein-N and another polynucleotide encodes a fragment of the base editor fused to an intein-C. Methods for designing and using inteins are known in the art and described, for example by WO2014004336, WO2017132580, WO2013045632A1, US20150344549, and US20180127780, each of which is incorporated herein by reference in their entirety.

In some embodiments, an ABE was split into N- and C-terminal fragments at Ala, Ser, Thr, or Cys residues within selected regions of SpCas9. These regions correspond to loop regions identified by Cas9 crystal structure analysis.

The N-terminal fragment is fused at the C-terminus to an intein-N and the C-terminal fragment is fused to an intein-C at an N-terminal amino acid selected from the group consisting of S303, T310, T313, S355, A456, S460, A463, T466, S469, T472, T474, C574, S577, A589, and S590, referenced to SEQ ID NO: 197. In various embodiments, the SpCas9 is split between amino acid positions 302 and 303, 309 and 310, 312 and 313, 354 and 355, 455 and 456, 459 and 460, 462 and 463, 465 and 466, 468 and 469, 471 and 472, 473 and 474, 573 and 574, 576 and 577, 588 and 589, or 589 and 590, referenced to SEQ ID NO: 197 to yield an N-terminal fragment and a C-terminal fragment, where the N-terminal fragment is fused at the C-terminus to a an intein-N and where the C-terminal fragment is fused at the N-terminus to an intein-C.

### Pharmaceutical Compositions

In some aspects, the present disclosure provides a pharmaceutical composition comprising any of the cells, polynucleotides, vectors, base editors, base editor systems, guide polynucleotides, fusion proteins, complexes, or the fusion protein-guide polynucleotide complexes described herein.

The pharmaceutical compositions of the present disclosure can be prepared in accordance with known techniques. See, e.g., Remington, The Science And Practice of Pharmacy (21st ed. 2005). In general, the cell, or population thereof is admixed with a suitable carrier prior to administration or storage, and in some embodiments, the pharmaceutical composition further comprises a pharmaceutically acceptable carrier. Suitable pharmaceutically acceptable carriers generally comprise inert substances that aid in administering the pharmaceutical composition to a subject, aid in processing the pharmaceutical compositions into deliverable preparations, or aid in storing the pharmaceutical composition prior to administration. Pharmaceutically acceptable carriers can include agents that can stabilize, optimize or otherwise alter the form, consistency, viscosity, pH, pharmacokinetics, solubility of the formulation. Such agents include buffering agents, wetting agents, emulsifying agents, diluents, encapsulating agents, and skin penetration enhancers. For example, carriers can include, but are not limited to, saline, buffered saline, dextrose, arginine, sucrose, water, glycerol, ethanol, sorbitol, dextran, sodium carboxymethyl cellulose, and combinations thereof.

In some embodiments, the pharmaceutical composition is formulated for delivery to a subject. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally to a diseased site (e.g., a liver). In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In some embodiments, any of the fusion proteins, gRNAs, and/or complexes described herein are provided as part of a pharmaceutical composition. In some embodiments, the pharmaceutical composition comprises any of the fusion proteins or complexes provided herein. In some embodiments pharmaceutical composition comprises a gRNA, a nucleic acid programmable DNA binding protein, a cationic lipid, and a pharmaceutically acceptable excipient. In embodiments, pharmaceutical compositions comprise a lipid nanoparticle and a pharmaceutically acceptable excipient. In embodiments, the lipid nanoparticle contains a gRNA, a base editor, a complex, a base editor system, or a component thereof of the present disclosure, and/or one or more polynucleotides encoding the same. Pharmaceutical compositions can optionally comprise one or more additional therapeutically active substances.

The compositions, as described above, can be administered in effective amounts. The effective amount will depend upon the mode of administration, the particular condition being treated, and the desired outcome. It may also depend upon the stage of the condition, the age and physical condition of the subject, the nature of concurrent therapy, if any, and like factors well-known to the medical practitioner. For therapeutic applications, it is that amount sufficient to achieve a medically desirable result.

In some embodiments, compositions in accordance with the present disclosure can be used for treatment of any of a variety of diseases, disorders, and/or conditions.

### Methods of Treatment

Some aspects of the present disclosure provide methods of treating a subject in need, the method comprising administering to a subject in need an effective therapeutic amount of a pharmaceutical composition as described herein. More specifically, the methods of treatment include administering to a subject in need thereof a lipid nanoparticle containing a guide RNA molecule and an mRNA molecule encoding a base editor of the disclosure. In other embodiments, the methods of the disclosure comprise expressing or introducing into a cell a base editor polypeptide and one or more guide RNAs capable of targeting a nucleic acid molecule encoding at least one polypeptide.

One of ordinary skill in the art would recognize that multiple administrations of the pharmaceutical compositions contemplated in particular embodiments may be required to affect the desired therapy. For example, a composition may be administered to the subject 1, 2, 3, 4, 5, 6, 7, 8, 9, or 10 or more times over a span of 1 week, 2 weeks, 3 weeks, 1 month, 2 months, 3 months, 4 months, 5 months, 6 months, 1 year, 2 years, 5, years, 10 years, or more.

Administration of the pharmaceutical compositions contemplated herein may be carried out using conventional techniques including, but not limited to, infusion, transfusion, or parenterally. In some embodiments, parenteral administration includes infusing or injecting intravascularly, intravenously, intramuscularly, intraarterially, intrathecally, intratumorally, intradermally, intraperitoneally, transtracheally, subcutaneously, subcuticularly, intraarticularly, subcapsularly, subarachnoidly and intrasternally.

### Kits

The disclosure provides kits for the treatment of phenylketonuria in a subject. In some embodiments, the kit further includes a base editor system or a polynucleotide encoding a base editor system, wherein the base editor polypeptide system a nucleic acid programmable DNA binding protein (napDNAbp), a deaminase, and a guide RNA. In some embodiments, the napDNAbp is Cas9 or Cas12. In some embodiments, the polynucleotide encoding the base editor is a mRNA sequence. In some embodiments, the deaminase is a cytidine deaminase or an adenosine deaminase. In some embodiments, the kit comprises an edited cell and instructions regarding the use of such cell.

The kits may further comprise written instructions for using a base editor, base editor system and/or edited cell as described herein. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit comprises instructions in the form of a label or separate insert (package insert) for suitable operational parameters. In yet another embodiment, the kit comprises one or more containers with appropriate positive and negative controls or control samples, to be used as standard(s) for detection, calibration, or normalization. The kit can further comprise a second container comprising a pharmaceutically-acceptable buffer, such as (sterile) phosphate-buffered saline, Ringer's solution, or dextrose solution. It can further include other materials desirable from a commercial and user standpoint, including other buffers, diluents, filters, needles, syringes, and package inserts with instructions for use.

The practice of embodiments of the present disclosure employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the disclosure, and, as such, may be considered in making and practicing embodiments of the disclosure. Particularly useful techniques for particular embodiments will be discussed in the sections that follow.

The following examples are put forth so as to provide those of ordinary skill in the art with a complete disclosure and description of how to make and use the assay, screening, and therapeutic methods of the disclosure, and are not intended to limit the scope of what the inventors regard as their invention.

### EXAMPLES

### Example 1: Correction of a Pathogenic Nucleobase in a Phenylalanine Hydroxylase (PAH) Polynucleotide Using Base Editor Systems

Experiments were undertaken to demonstrate that base editor systems of the disclosure are effective in correcting a pathogenic nucleobase in a PAH polynucleotide in GM02406 cells from a phenylketonuria patient.

GM02406 cells are fibroblast cells from a white male Homo sapiens patient that had phenylketonuria. The GM02406 cells were obtained from the NIGMS Human Genetic Cell Repository. The subject from which the GM02406 cells were obtained had the following attributes: clinically affected; pyloric stenosis; normal level of dihydropteridine reductase in skin fibroblasts; compound heterozygote; one PAH allele had a T>G transversion at nucleotide 896 in Exon 8 (896T>G) resulting in a substitution of cysteine for phenylalanine at codon 299 (Phe299Cys (F299C)) and a second allele had a C>T transition at nucleotide 1222 in Exon 11 (1222C>T) resulting in a substitution of tryptophan for arginine at codon 408 (Arg408Trp).

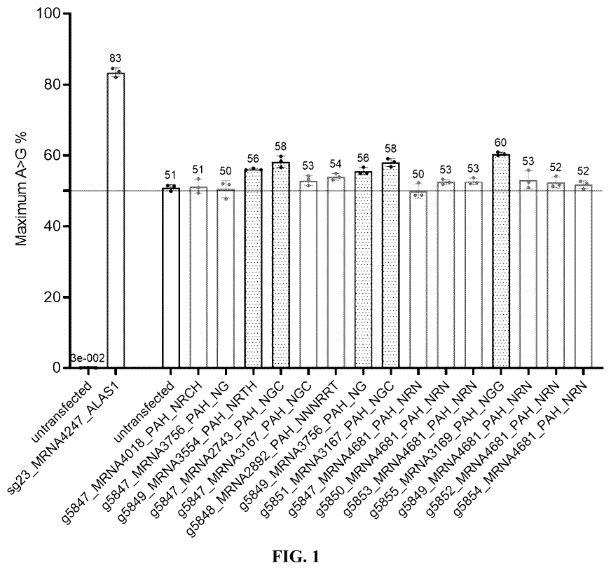

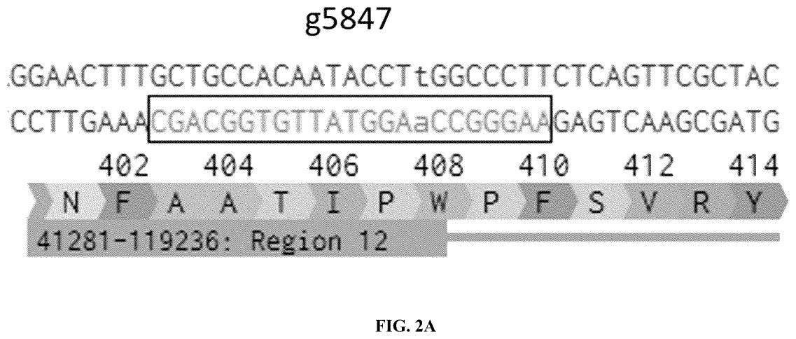

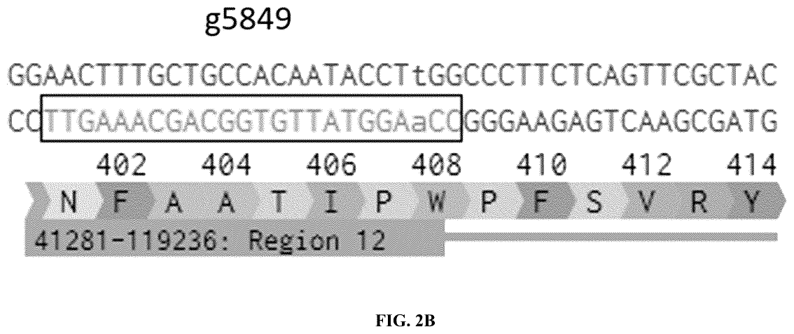

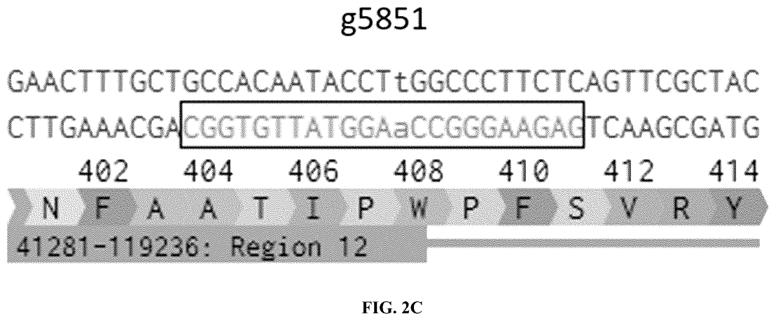

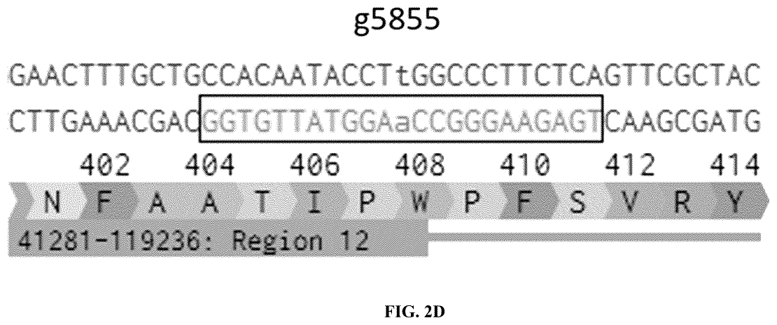

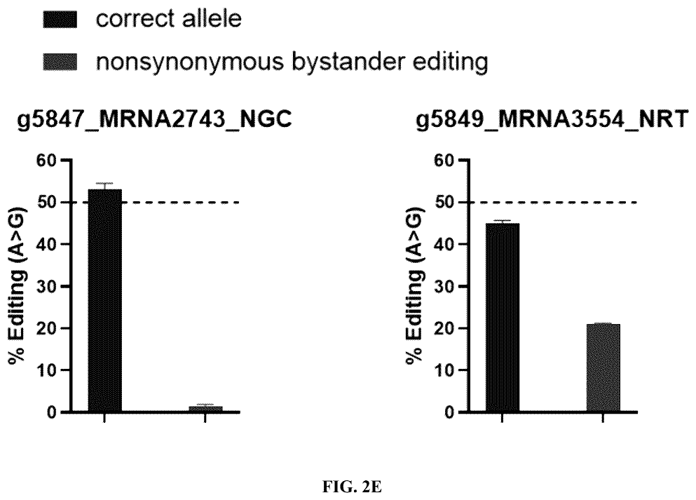

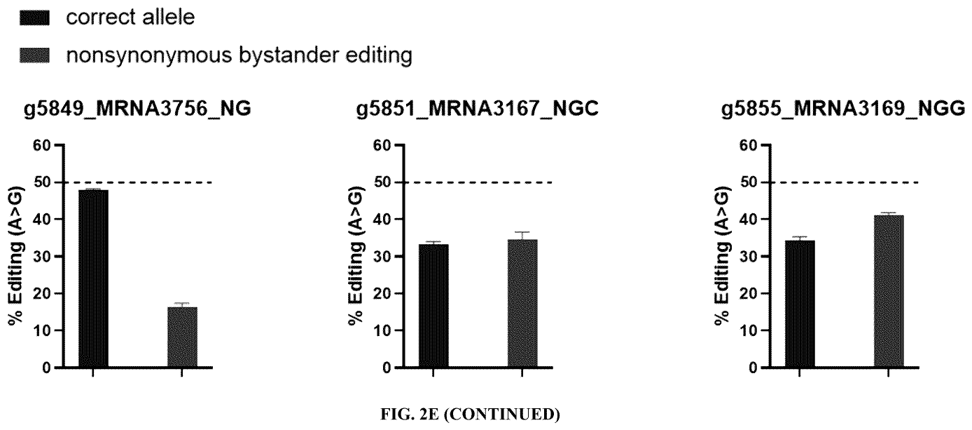

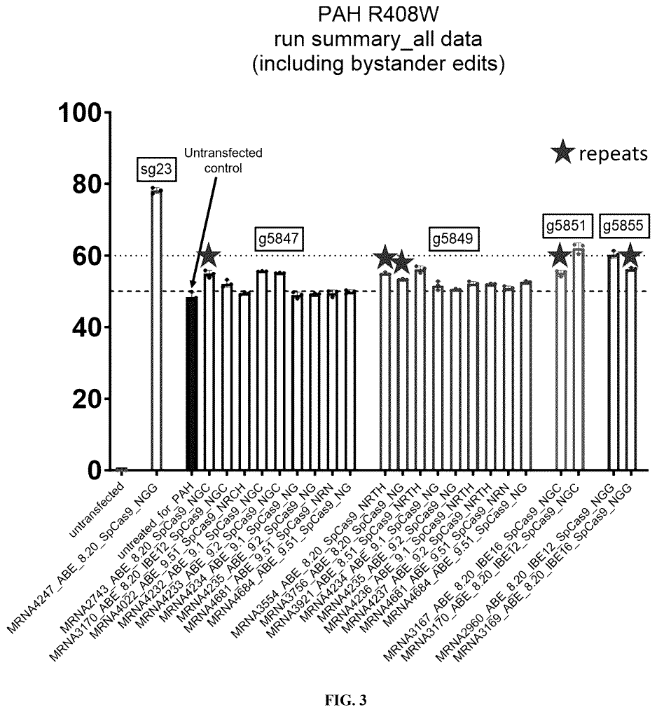

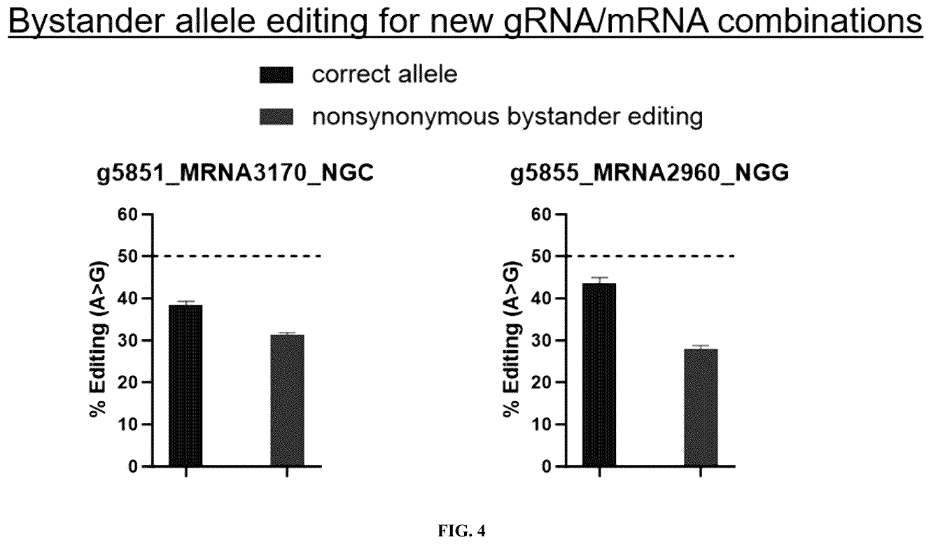

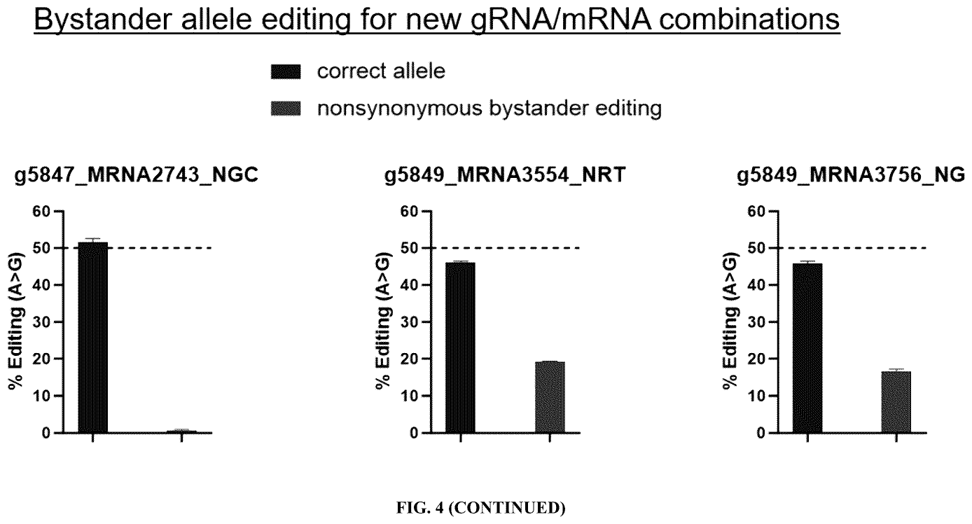

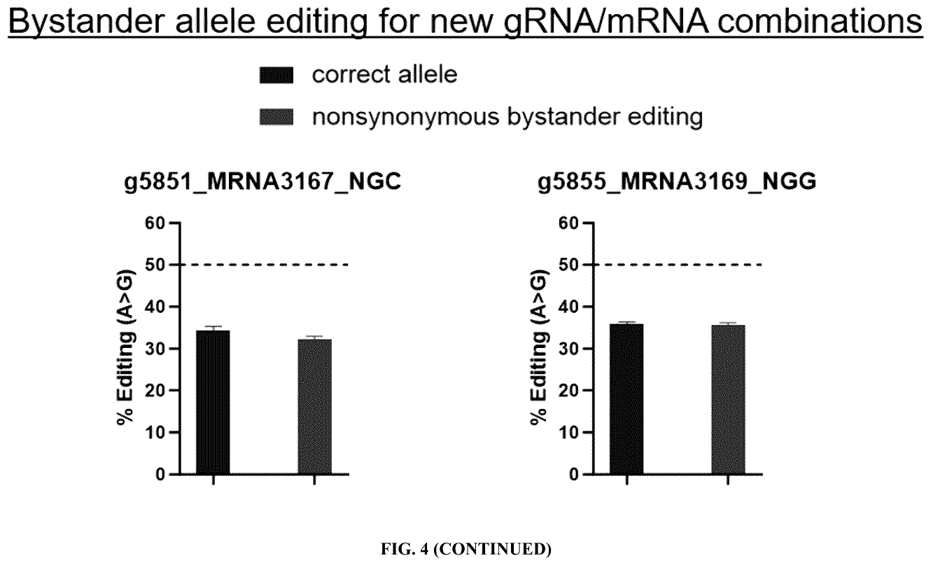

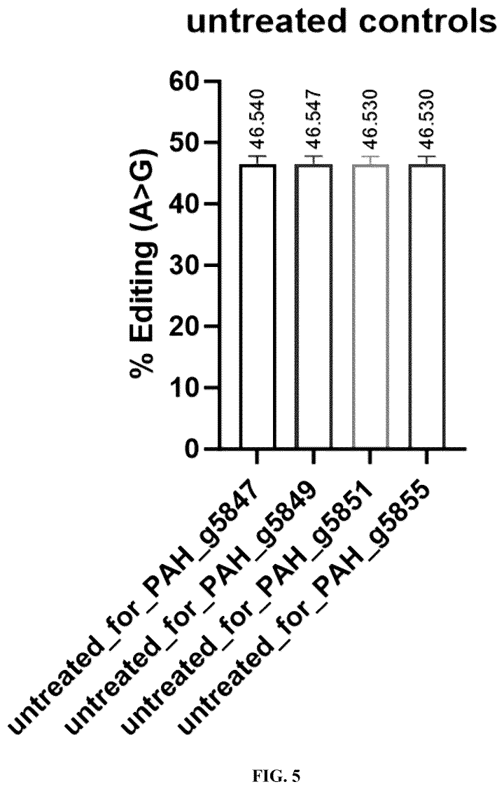

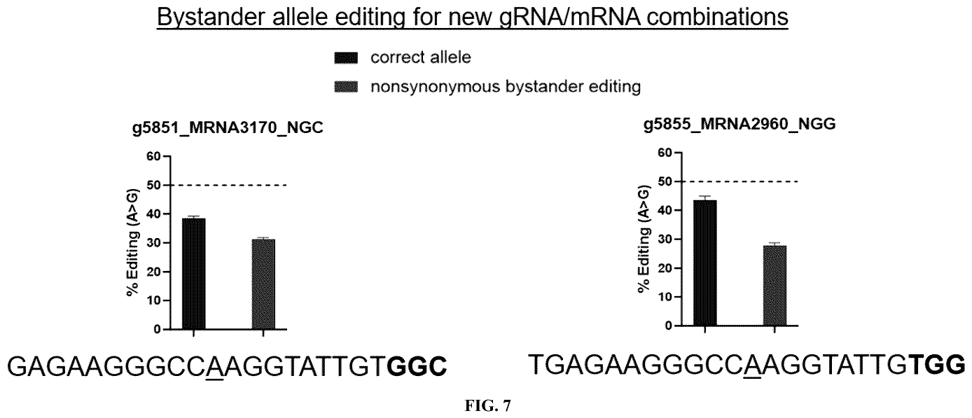

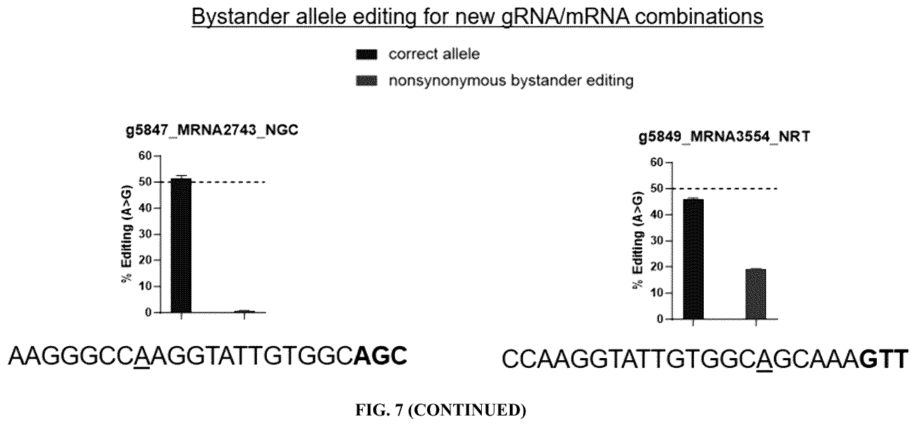

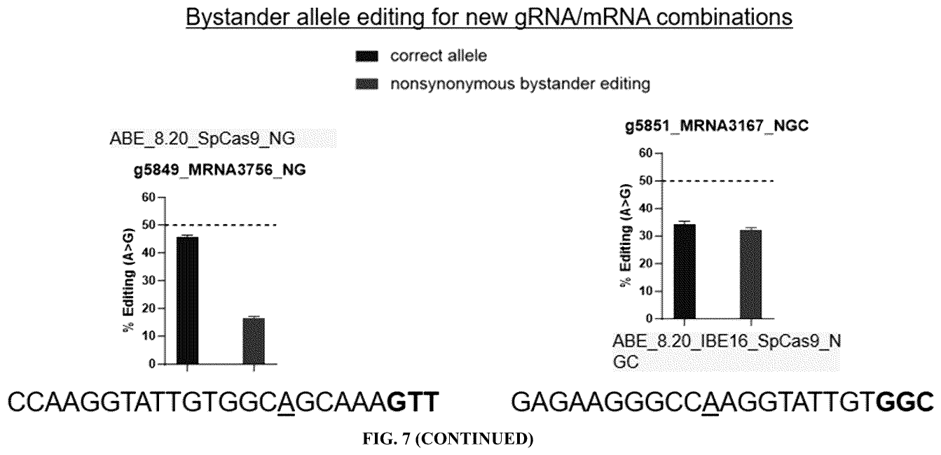

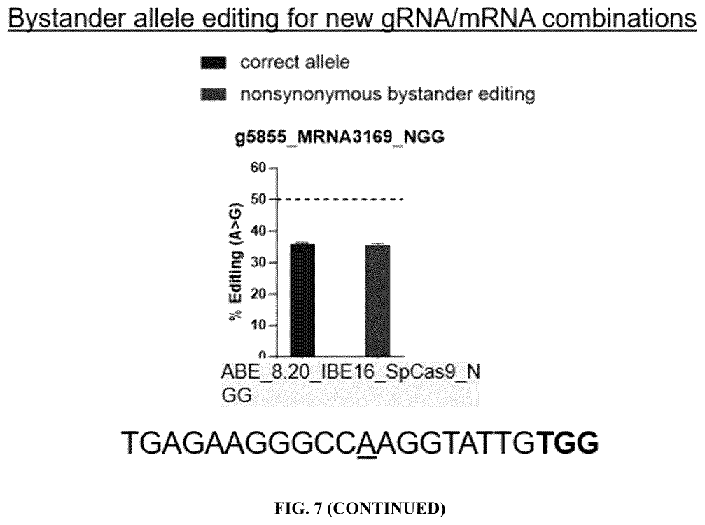

To evaluate base editing in the cells, about 6×10e4 GM02406 cells were transfected with base editor systems containing 1000 ng mRNA encoding a base editor containing an adenosine deaminase domain and 33 ng of a guide RNA targeting the base editor to deaminate a target nucleobase in a PAH polynucleotide. At about 72 hours following transfection, on-target and bystander percent A to G base editing was measured in the cells using next-generation sequencing (FIGS. 1, 2E, 3, 4, 5, and 7). The sequences targeted for base editing by each guide polynucleotide are depicted in FIGS. 2A-2E and 7.

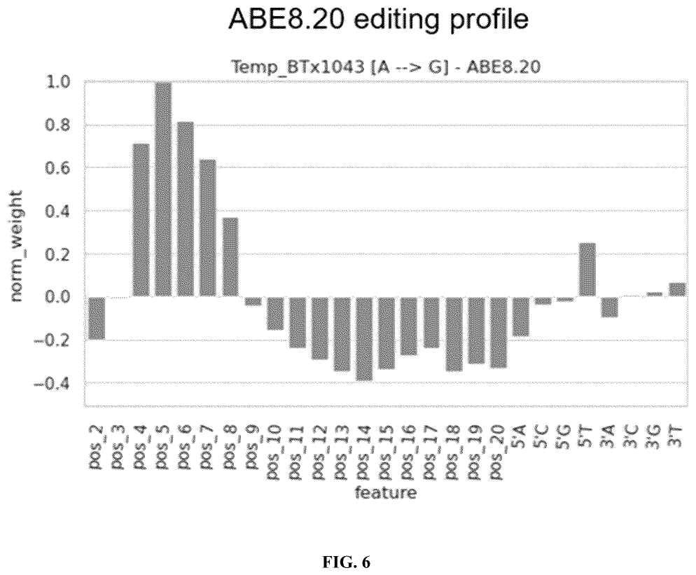

In an effort to identify alternative napDNAbp domains suitable for use in base editor systems for correcting a pathogenic PAH nucleotide, the base editing profile of ABE8.20 (FIG. 6) was taken into consideration as well as the following target sites, where the PAM sequence is shown in hold and the target nucleobase is underlined.

1. Position the target A at pos 4:
(SEQ ID NO: 650)
GCCAA GGTAT TGTGG CAGCA AAGTTC
2. Position the target A at pos 5:
(SEQ ID NO: 651)
GGCCAAGGTA TTGTG GCAGC AAA GTT
3. Position the target A at pos 6:
(SEQ ID NO: 652)
GGGCC AAGGT ATTGT GGCAG CAAAGT
4. Position the target A at pos 7:
(SEQ ID NO: 653)
AGGGC CAAGG TATTG TGGCA GCA AAG

It was then determined that the napDNAbp domains listed in Table 8 below would be suitable for use in editing a target adenosine (A) identified in the above target sites numbered 1-5:

| Target A pos | Option 1 | Option 2 |
|---|---|---|
| 4 | NRN SpCas9 | NAG Nme2Cas9 (see Huang, et al., |
| | (see Walton, et al, | Nature Biotechnology, 41: 96-107 |
| | Science, 368: 290- | (2023)) |
| | 296 (2020)) | |
| 5 | NAA iSpymac | NNVGTT |
| | (see Chatterjee, et | SaCas9 |
| | al. Nature | (see Ma, et al., Nature |
| | Communications, | Communications, 10: 560 (2019)) |
| | 11: 2474 (2020)) | |
| 6 | NAA iSpymac | NNNRRT |
| | (see Chatterjee, et | SaCas9* |
| | al. Nature | |
| | Communications, | |
| | 11: 2474 (2020)) | |
| 7 | NCA | NNVRRN |
| | NmeCas9 | SaCas9 |
| | (see Huang, et al., | (see Ma, et al., Nature |
| | Nature | Communications, 10: 560 (2019)) |
| | Biotechnology, | |
| | 41: 96-107 (2023)) | |
| 8 | MEW-NGC Cas9 | IBE-NGC*, where “IBE” indicates an |
| | | internal base editor |

Base editing was found to be effective in correcting the pathogenic PAH nucleobases.

### Other Embodiments

From the foregoing description, it will be apparent that variations and modifications may be made to the aspects or embodiments described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference. The present disclosure may be related to Brooks, et al. “Rapid and definitive treatment of phenylketonuria in variant-humanized mice with corrective editing,” Nature Communications, 14:3451 (2023), doi.org/10.1038/s41467-023-39246-2, the disclosure of which is incorporated herein by reference in its entirety for all purposes.

What is claimed:

1. A method of editing a nucleobase of an phenylalanine hydroxylase (PAH) polynucleotide, the method comprising contacting the PAH polynucleotide with a guide RNA, or a polynucleotide encoding the guide RNA, and a base editor comprising a fusion protein or a protein complex comprising a nucleic acid programmable DNA binding protein (napDNAbp) domain and a deaminase domain, or one or more polynucleotides encoding the base editor, wherein said guide RNA targets said base editor to effect an alteration to a nucleobase in codon 408 of the PAH polynucleotide, wherein the deaminase domain comprises or consists of the following amino acid sequence with one or more amino acid alterations MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a fragment thereof lacking only the N-terminal methionine, wherein the one or more amino acid alterations comprises or consists of a set of alterations selected from the group consisting of:
a) I76Y, V82T, Y123H, Y147R, and Q154R;
b) I76Y, V82T, Y123H, Y147R, F149Y, and Q154R; and
c) I76Y, V82T, Y123H, Y147D, F149Y, Q154R, T166I, and D167N.

2. The method of claim 1, wherein the alteration to the nucleobase in codon 408 of the PAH polynucleotide results in a W408R amino acid alteration in a PAH polypeptide encoded by the PAH polynucleotide.

3. The method of claim 1, wherein the base editor comprises an amino acid sequence having at least 85% sequence identity to a sequence selected from those listed in Table 2.

4. The method of claim 1, wherein the guide RNA comprises a spacer comprising at least 10 contiguous nucleotides of a spacer sequence selected from those listed in Table 1.

5. The method of claim 1, wherein the one or more polynucleotides encoding the base editor comprise an RNA sequence encoding a polypeptide having at least 85% sequence identity to an amino acid sequence selected from those listed in Table 2.

6. A method of treating phenylketonuria (PKU) in a subject in need thereof, the method comprising administering to a cell of the subject a base editor system comprising a base editor comprising a fusion protein or protein complex comprising a nucleic acid programmable DNA binding protein (napDNAbp) domain and a deaminase domain, or one or more polynucleotides encoding the base editor, and a guide RNA that targets the base editor to effect an alteration to a nucleobase in codon 408 of a phenylalanine hydroxylase (PAH) polynucleotide in the cell, or a polynucleotide encoding the guide RNA, thereby treating hypertension in the subject, wherein the deaminase domain comprises or consists of the following amino acid sequence with one or more amino acid alterations MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a fragment thereof lacking only the N-terminal methionine, wherein the one or more amino acid alterations comprises or consists of a set of alterations selected from the group consisting of:
a) I76Y, V82T, Y123H, Y147R, and Q154R;
b) I76Y, V82T, Y123H, Y147R, F149Y, and Q154R; and
c) I76Y, V82T, Y123H, Y147D, F149Y, Q154R, T166I, and D167N.

7. The method of claim 6, wherein the alteration to the nucleobase in codon 408 of the PAH polynucleotide results in a W408R amino acid alteration in a PAH polypeptide encoded by the PAH polynucleotide.

8. The method of claim 6, wherein the base editor comprises an amino acid sequence having at least 85% sequence identity to a sequence selected from those listed in Table 2.

9. The method of claim 6, wherein the guide RNA comprises a spacer comprising at least 10 contiguous nucleotides of a spacer sequence selected from those listed in Table 1.

10. The method of claim 6, wherein the one or more polynucleotides encoding the base editor comprise an RNA sequence encoding a polypeptide having at least 85% sequence identity to an amino acid sequence selected from those listed in Table 2.

11. A modified cell comprising an alteration in a nucleobase of an PAH polynucleotide, wherein the alteration is prepared by the method of claim 1, and wherein the alteration increases activity of the encoded PAH polypeptide as compared to a control cell without the alteration.

12. A base editor system comprising a base editor or one or more polynucleotides encoding the base editor, wherein the base editor comprises a nucleic acid programmable DNA binding protein domain (napDNAbp) and a deaminase domain, and a guide RNA that targets said base editor to effect an alteration to a nucleobase in codon 408 of a PAH polynucleotide, wherein the deaminase domain comprises or consists of the following amino acid sequence with one or more amino acid alterations MSEVEFSHEYWMRHALTLAKRARDEREVPVGAVLVLNNRVIGEGWNRAIGLHDPTAHAEIMALR QGGLVMQNYRLIDATLYVTFEPCVMCAGAMIHSRIGRVVFGVRNAKTGAAGSLMDVLHYPGMNH RVEITEGILADECAALLCYFFRMPRQVFNAQKKAQSSTD (SEQ ID NO: 1), or a fragment thereof lacking only the N-terminal methionine, wherein the one or more amino acid alterations comprises or consists of a set of alterations selected from the group consisting of:
a) I76Y, V82T, Y123H, Y147R, and Q154R;
b) I76Y, V82T, Y123H, Y147R, F149Y, and Q154R; and
c) I76Y, V82T, Y123H, Y147D, F149Y, Q154R, T166I, and D167N.

13. The base editor system of claim 12, wherein the alteration to the nucleobase in codon 408 of the PAH polynucleotide results in a W408R amino acid alteration in a PAH polypeptide encoded by the PAH polynucleotide.

14. The base editor system of claim 12, wherein the base editor comprises an amino acid sequence having at least 85% sequence identity to a sequence selected from those listed in Table 2.

15. The base editor system of claim 12, wherein the guide RNA comprises a spacer comprising at least 10 contiguous nucleotides of a spacer sequence selected from those listed in Table 1.

16. The base editor system of claim 12, wherein the one or more polynucleotides encoding the base editor comprise an RNA sequence encoding a polypeptide having at least 85% sequence identity to an amino acid sequence selected from those listed in Table 2.

17. A set of polynucleotides encoding the base editor system of claim 12, or a component thereof.

18. A lipid nanoparticle comprising the base editor system of claim 12.

19. A pharmaceutical composition comprising the base editor system of claim 12, and a pharmaceutically acceptable excipient.

20. A kit comprising the base editor system comprising the base editor system of claim 12, wherein the kit further comprises a container.

21. A guide RNA comprising a sequence listed in Table 1.
